(19) United States
(12) Patent Application Publication
(10) Pub. No.: US 2010/0311581 A1
(43) Pub. Date: Dec. 9, 2010

(54) Azolylmethyloxiranes, use Thereof and Agents Containing the Same

(75) Inventors: Jochen Dietz, Karlsruhe (DE); Thomas Grote, Wachenheim (DE); Bernd Mueller, Frankenthal (DE); Jan Klaas Lohmann, Ludwigshafen (DE); Jens Renner, Bad Duerkheim (DE); Sarah Ulmschneider, Bad Duerkheim (DE); Alice Glaettli, Frankfurt (DE); Marianna Vrettou, Mannheim (DE)

Correspondence Address: BRINKS, HOFER, GILSON & LIONE, P.O. BOX 110285, RESEARCH TRIANGLE PARK, NC, 27709, US

(73) Assignee: BASF SE, Ludwigshafen (DE)

(21) Appl. No.: 12/808,797

(22) PCT Filed: Dec. 15, 2008
(86) PCT No.: PCT/EP2008/067483

§ 371 Date: Jun. 17, 2010

(30) Foreign Application Priority Data
Dec. 19, 2007 (EP) 07123688.9

(51) Int. Cl. A01C 1/06; C07D 405/06; A01N 43/653; A01N 57/24; C07D 301/00; C07D 303/36; C07D 249/08; C07D 303/08; A01P 3/00; A01P 17/00

(52) U.S. Cl. 504/100; 548/263.2; 548/264.2; 548/264.4; 548/118; 514/384; 514/93; 549/513; 549/551; 548/262.2; 549/563; 504/139

(57) ABSTRACT

The present invention relates to triazolylmethyloxiranes of the formula I

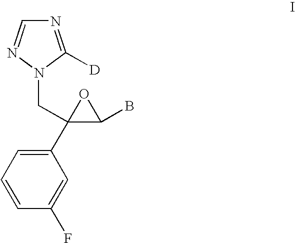

in which the variables D and B have the meanings as defined in the description and the claims.

The present invention relates to azolylmethyloxiranes of the formula I

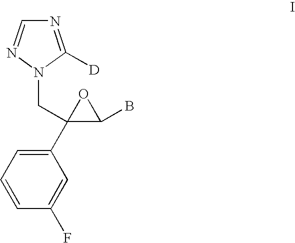

in which the variables have the following meanings:
B is phenyl which is unsubstituted or substituted by one, two, three or four identical or different substituents L, where L is as defined below:
   L is halogen, cyano, nitro, cyanato (OCN), C₁-C₈-alkyl, C₁-C₈-haloalkyl, phenyl-C₁-C₆-alkyloxy, C₂-C₈-alkenyl, C₂-C₈-haloalkenyl, C₂-C₈-alkynyl, C₂-C₈-haloalkynyl, C₄-C₁₀-alkadienyl, C₄-C₁₀-halo-alkadienyl, C₁-C₈-alkoxy, C₁-C₈-haloalkoxy, C₁-C₈-alkylcarbonyloxy, C₁-C₈-alkylsulfonyloxy, C₂-C₈-alkenyloxy, C₂-C₈-haloalkenyloxy, C₂-C₈-alkynyloxy, C₂-C₈-haloalkynyloxy, C₃-C₈-cycloalkyl, C₃-C₈-halocycloalkyl, C₃-C₈-cycloalkenyl, C₃-C₈-halocycloalkenyl, C₃-C₈-cycloalkoxy, C₃-C₆-cycloalkenyloxy, hydroxyimino-C₁-C₈-alkyl, C₁-C₆-alkylene, oxy-C₂-C₄-alkylene, oxy-C₁-C₃-alkyleneoxy, C₂-C₈-alkenyloximino-C₁-C₈-alkyl, C₂-C₈-alkynyloximino-C₁-C₈-alkyl, S(═O)ₙA¹, C(═O)A², C(═S)A², NA³A⁴, phenyl, phenyloxy or a five- or six-membered saturated, partially unsaturated or aromatic heterocycle which comprises one, two, three or four heteroatoms from the group consisting of O, N and S; where n, A¹, A², A³, A⁴ are as defined below:
   n is 0, 1 or 2;
   A¹ is hydrogen, hydroxyl, C₁-C₈-alkyl, C₁-C₈-haloalkyl, amino, C₁-C₈-alkylamino or di-C₁-C₈-alkylamino,
   A² is one of the groups mentioned for A¹ or C₂-C₈-alkenyl, C₂-C₈-haloalkenyl, C₂-C₈-alkynyl, C₂-C₈-haloalkynyl, C₁-C₈-alkoxy, C₁-C₈-haloalkoxy, C₂-C₈-alkenyloxy, C₂-C₈-haloalkenyloxy, C₂-C₈-alkynyloxy, C₂-C₈-haloalkynyloxy, C₃-C₈-cycloalkyl, C₃-C₈-halocycloalkyl, C₃-C₈-cycloalkoxy or C₃-C₈-halocycloalkoxy;
   A³, A⁴ independently of one another are hydrogen, C₁-C₈-alkyl, C₁-C₈-haloalkyl, C₂-C₈-alkenyl, C₂-C₈-haloalkenyl, C₂-C₈-alkynyl, C₂-C₈-haloalkynyl, C₃-C₈-cycloalkyl, C₃-C₈-halocycloalkyl, C₃-C₈-cycloalkenyl or C₃-C₈-halocycloalkenyl;
   where the aliphatic and/or alicyclic and/or aromatic groups of the radical definitions of L for their part may carry one, two, three or four identical or different groups R^{L}:
   R^{L} is halogen, cyano, nitro, C₁-C₈-alkyl, C₁-C₈-haloalkyl, C₁-C₈-alkoxy, C₁-C₈-haloalkoxy, C₃-C₈-cycloalkyl, C₃-C₈-halocycloalkyl, C₃-C₈-cycloalkenyl, C₃-C₈-cycloalkoxy, C₃-C₈-halocycloalkoxy, C₁-C₈-alkylcarbonyl, C₁-C₈-alkylcarbonyloxy, C₁-C₈-alkoxycarbonyl, amino, C₁-C₈-alkylamino, di-C₁-C₈-alkylamino;
D -is S—R, where
   R is hydrogen, C₁-C₈-alkyl, C₁-C₈-haloalkyl, C₂-C₈-alkenyl, C₂-C₈-haloalkenyl, C₂-C₈-alkynyl, C₂-C₈-haloalkynyl, C(═O)R³, C(═S)R³, SO₂R⁴ or CN; where
   R³ is hydrogen, C₁-C₈-alkyl, C₁-C₈-haloalkyl, C₁-C₈-alkoxy, C₁-C₈-haloalkoxy or NA³A⁴; and
   R⁴ is C₁-C₈-alkyl, phenyl-C₁-C₈-alkyl or phenyl, where the phenyl groups are in each case unsubstituted or substituted by one, two or three groups independently selected from the group consisting of halogen and C₁-C₄-alkyl;
is a group DI

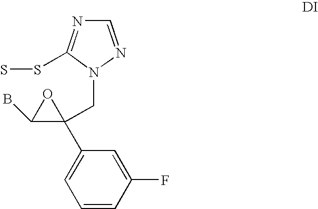

where B is as defined above;
   is a group DII

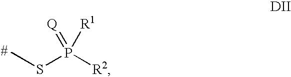

where # is the point of attachment to the triazolyl ring and Q, R¹ and R² are as defined below:
Q is O or S;

R¹, R² independently of one another are C₁-C₈-alkyl, C₁-C₈-haloalkyl, C₁-C₈-alkoxy, C₁-C₈-alkoxy-C₁-C₈-alkoxy, C₁-C₈-haloalkoxy, C₁-C₈-alkoxy-C₁-C₈-alkyl, C₁-C₈-alkylthio, C₂-C₈-alkenylthio, C₂-C₈-alkynyl-thio, C₃-C₈-cycloalkyl, C₃-C₈-cycloalkylthio, phenyl, phenyl-C₁-C₄-alkyl, phenoxy, phenylthio, phenyl-C₁-C₄-alkoxy or NR⁵R⁶, where R⁵ is H or C₁-C₈-alkyl and R⁶ is C₁-C₈-alkyl, phenyl-C₁-C₄-alkyl or phenyl or R⁵ and R⁶ together are an alkylene chain having four or five carbon atoms or form a radical of the formula —CH₂—CH₂—O—CH₂—CH₂— or —CH₂—CH₂—NR⁷—CH₂—CH₂— in which R⁷ is hydrogen or C₁-C₄-alkyl; where the aromatic groups in the radicals mentioned above are in each case independently of one another unsubstituted or substituted by one, two or three groups selected from the group consisting of halogen and C₁-C₄-alkyl;
or
   is a group SM, where M is as defined below:
M is an alkali metal cation, an equivalent of an alkaline earth metal cation, an equivalent of a copper, zinc, iron or nickel cation or an ammonium cation of the formula (E)

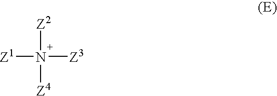

in which
   Z¹ and Z² independently are hydrogen or C₁-C₈-alkyl;
   Z³ and Z⁴ independently are hydrogen, C₁-C₈-alkyl, benzyl or phenyl; where the phenyl groups are in each case unsubstituted or substituted by one, two or three groups independently selected from the group consisting of halogen and C₁-C₄-alkyl;
and an agriculturally acceptable salt thereof.

The compounds of the formula I can be present in the “thiol” form of the formula Ia or in the “thiono” form of the formula Ib:

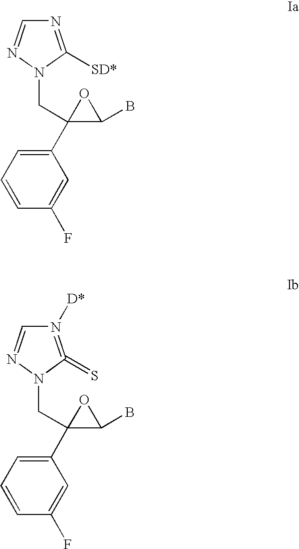

in which D* is:
R¹⁰, where R¹⁰ has the meaning defined above;
a group DII*

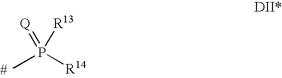

where # is the point of attachment to the sulfur atom in formula Ia or azolyl ring in formula Ib and Q, R¹³ and R¹⁴ have the meaning defined above; or
   a group M, where M has the meaning defined above,
      and in which the remaining substituents have the meaning defined above.

However, for the sake of simplicity, in each case generally only the “thiol” form is shown here.

The invention furthermore relates to the preparation of the compounds I, to the intermediates for preparing the compounds I and to their preparation, and also to the use of the compounds according to the invention for controlling phytopathogenic fungi, and to compositions comprising them.

Triazolylmethyloxiranes having a substituted triazole group are known, for example, from WO 96/38440, WO 97/41107, WO 97/42178, WO 97/43269, WO 97/44331, WO 97/443332, WO 99/05149 and WO 99/21853.

However, in particular at low application rates, the fungicidal action of the compounds known from the prior art is sometimes unsatisfactory. Accordingly, it was an object of the present invention to provide novel compounds which preferably have improved properties, such as improved fungicidal action and/or better toxicological properties. Surprisingly, this object was achieved with the compounds of the formula I described here.

Owing to the basic character of their nitrogen atoms, the compounds I are capable of forming salts or adducts with inorganic or organic acids or with metal ions. This also applies to most of the precursors described herein of compounds I, the salts and adducts of which are also provided by the present invention.

Examples of inorganic acids are hydrohalic acids, such as hydrogen fluoride, hydrogen chloride, hydrogen bromide and hydrogen iodide, carbonic acid, sulfuric acid, phosphoric acid and nitric acid.

Suitable organic acids are, for example, formic acid and alkanoic acids, such as acetic acid, trifluoroacetic acid, trichloroacetic acid and propionic acid, and also glycolic acid, thiocyanic acid, lactic acid, succinic acid, citric acid, benzoic acid and other arylcarboxylic acids, cinnamic acid, oxalic acid, alkylsulfonic acids (sulfonic acids having straight-chain or branched alkyl radicals of 1 to 20 carbon atoms), arylsulfonic acids or aryldisulfonic acids (aromatic radicals, such as phenyl and naphthyl, which carry one or two sulfonic acid groups), alkylphosphonic acids (phosphonic acids having straight-chain or branched alkyl radicals of 1 to 20 carbon atoms), arylphosphonic acids or aryldiphosphonic acids (aromatic radicals, such as phenyl and naphthyl, which carry one or two phosphoric acid radicals), where the alkyl or aryl radicals may carry further substituents, for example p-toluenesulfonic acid, salicylic acid, p-aminosalicylic acid, 2-phenoxybenzoic acid, 2-acetoxybenzoic acid etc.

Suitable metal ions are in particular the ions of the elements of the second main group, in particular calcium and magnesium, of the third and fourth main group, in particular aluminum, tin and lead and also of the elements of transition groups one to eight, in particular chromium, manganese, iron, cobalt, nickel, copper, zinc and others. Particular preference is given to the metal ions of the elements of transition groups of the fourth period. The metals can be present in the various valencies that they can assume.

The compounds of the formula I according to the invention can be prepared by different routes analogously to processes known per se of the prior art (see, for example, the prior art cited at the outset and Pflanzenschutz-Nachrichten Bayer 57/2004, 2, pages 145-162). The compounds according to the invention can be prepared, for example, according to the syntheses shown in the schemes below.

The compounds according to the invention can be prepared in an advantageous manner from compounds of the formula II

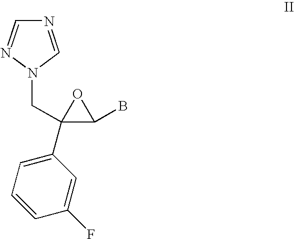

in which B is as defined herein by reaction with a strong base and sulfur powder. This results in the formation of compounds of the formula I in which D is SH (compounds I-1):

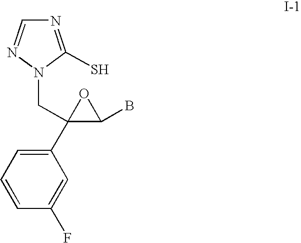

Suitable bases are all bases known to the person skilled in the art as being suitable for such reactions. Preference is given to using strong alkali metal bases such as, for example, n-butyllithium, lithium diisopropylamide, sodium hydride, sodium amide or potassium tert-butoxide. It may be preferred to carry out the reaction in the presence of an additive such as, for example, tetramethylethylenediamine (TMEDA).

Suitable solvents are all inert organic solvents customary for such reactions, where preferably ethers such as tetrahydrofuran, dioxane, diethyl ether and 1,2-dimethoxyethane or liquid ammonia or strongly polar solvents such as dimethyl sulfoxide may be used.

Sulfur is preferably used as a powder. For the hydrolysis, use is made of water, if appropriate in the presence of an organic or inorganic acid such as, for example, acetic acid, dilute sulfuric acid or dilute hydrochloric acid.

The reaction temperature is preferably between −70° C. and +20° C., in particular between −70° C. and 0° C. The reaction is generally carried out under atmospheric pressure.

In general, 1 to 3 equivalents, preferably 1 to 2.5 equivalents, of a strong base and then an equivalent amount or an excess of sulfur are employed per mole of the compound of the formula II. The reaction can be carried out under an atmosphere of protective gas such as, for example, under nitrogen or argon. Work-up is carried out according to procedures generally known to the person skilled in the art. Usually, the reaction mixture is extracted with a suitable organic solvent, and the residue is, if appropriate, purified by recrystallisation and/or chromatography.

It is also possible to prepare compounds I by direct reaction with sulfur, preferably sulfur powder, without the use of a strong base such as butyllithium.

A further possibility, starting from compounds II, of preparing compounds I according to the invention is to react compounds II with sulfur in the presence of an aprotic, polar solvent, such as, for example, an amide (such as dimethylformamide (DMF)) or N-alkylpyrrolidone (such as N-octylpyrrolidone, N-dodecylpyrrolidone or N-methyl-pyrrolidone (NMP)). See also WO 99/19307, WO 97/06151, WO 97/05119 and WO 96/41804.

The reaction is generally carried out at temperatures in the range from 140° C. to 160° C. The reaction components are customarily employed in amounts such that approximately 6 to 15 mol of sulfur are used to 1 mol of the compound II. Sulfur is generally employed in the form of powder. During the reaction, air is passed over the reaction mixture.

The compounds I according to the invention can moreover be advantageously prepared starting from compounds of the formula II by reaction with disulfides or dirhodan:

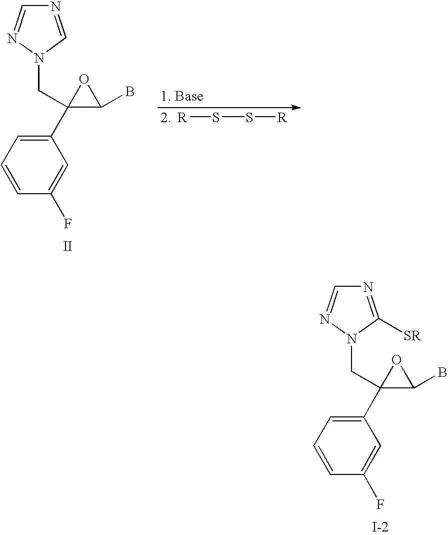

wherein B is defined as described herein, and R can be C1-C8-alkyl, C1-C8-haloalkyl, C2-C8-alkenyl, C2-C8-haloalkenyl, C2-C8-alkynyl, C2-C8-haloalkynyl or CN.

Possible bases are all suitable bases known to the person skilled in the art for such reactions. Preferably, strong alkali metal bases, such as, for example, n-butyllithium, lithium diisopropylamide, sodium hydride, sodium amide or potassium tert. butoxide are used. It may be preferable to carry out the reaction in the presence of an additive, such as, for example, tetramethylethylendiamine (TMEDA).

The disulfides are commercially obtainable or can be synthesized according to known preparation processes. A specific disulfide is dirhodan NC—S—S—CN.

Suitable solvents are all inert organic solvents customary for reactions of this type, wherein ethers such as tetrahydrofuran, dioxane, diethyl ether and 1,2-dimethoxyethane or liquid ammonia or strongly polar solvents such as dimethyl sulfoxide can preferably be used.

The reaction temperature is preferably between −70° C. and +20° C., in particular between −70° C. and 0° C. The reaction is in general carried out under normal pressure. In general, 1 to 3 equivalents, preferably 1 to 2.5 equivalents, of strong base and subsequently an equivalent amount or an excess of disulfide are employed to 1 mol of the compound of the formula II. The reaction can be carried out under a protective gas atmosphere, such as, for example, under nitrogen or argon. The work-up is carried out according to procedures generally known to the person skilled in the art. Customarily, the reaction mixture is extracted with a suitable organic solvent and the residue is if appropriate purified by recrystallization and/or chromatography.

By further reaction of compounds I-1 with R—X, where R is as defined herein at a different location and X is a leaving group such as, for example, halogen, such as Cl, Br or I, or trifluoro-C₁-C₆-alkylsulfonate, it is possible to prepare various compounds of the formula I according to the invention. To prepare compounds where D=SR where R=C₁-C₆-alkyl, preferably methyl or ethyl, a compound I-1 is reacted with the corresponding alkyl halide (see also WO 96/38440).

Compounds of the formula I in which D is S—C(═O)NA³A⁴ can be synthesized analogously to the process described in WO 99/21853.

Compounds of the formula I in which D is a group DII can be synthesized analogously to the process described in WO 99/05149.

Compounds of the formula I in which D is S—SO₂R⁴ can be synthesized analogously to the process described in WO 97/44332.

Compounds of the formula I in which D is S—CN can be synthesized analogously to the process described in WO 99/44331.

Compounds of the formula I in which D is a group DI can be synthesized analogously to the process described in WO 97/43269.

Compounds of the formula I in which D is a group S—C(═O)R³ where R³=C₁-C₈-alkyl, C₁-C₈-haloalkyl, C₁-C₈-alkoxy or C₁-C₈-haloalkoxy can be synthesized analogously to the process described in WO 97/42178.

Compounds of the formula I in which D is a group SM can be synthesized analogously to the process described in WO 97/41107.

The compounds of the formula II can be synthesized analogously to the prior art cited at the outset and/or the literature references cited therein. Some compounds II are described in PCT/EP2007/063213. On the other hand, some compounds of the formula II are novel, and these and their agriculturally acceptable salts also form part of the subject matter of the present invention. Compounds II also have fungicidal activity, and accordingly, the present invention also relates to the use of the compounds II and/or the salts thereof as fungicides.

Compounds of the formula III

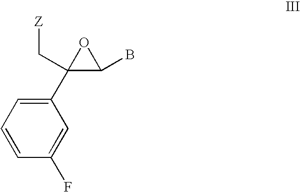

in which Z is a leaving group X (compounds III.1, see below) or OH (compounds III.2, see below) and B is as defined below are important starting compounds needed to obtain, finally, the compounds according to the invention.

Thus, compounds II can be prepared, for example, from compounds III.1

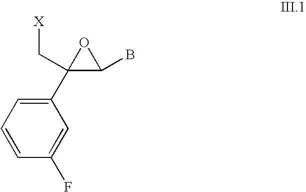

where X is a leaving group such as, for example, halogen (for example Cl or Br) or OSO₂R, where R is C₁-C₆-alkyl, C₁-C₆-haloalkyl, aryl or substituted aryl; OSO₂R is in particular a mesylate, triflate, phenyl or toluenesulfonate group. To obtain compounds of the formula II, compounds of the formula III.1 are reacted with 1,2,4-triazole and a base such as, for example, sodium hydride, for example in DMF. See also, for example, EP 0 421 125 A2.

Some compounds of the formula III.1 are novel. Accordingly, the invention also provides compounds of the formula III.1 in which B is as defined or as preferably defined for formula I and X is a leaving group, in particular halogen (for example Cl or Br) or OSO₂R in which R is C₁-C₆-alkyl, C₁-C₆-haloalkyl, aryl or substituted aryl, except for the compounds anti-2-(3-fluorophenyl)-2-(chloromethyl)-3-(2-chlorophenyl)oxirane, anti-2-(3-fluorophenyl)-2-(chloromethyl)-3-(4-chlorophenyl)oxirane, anti-2-(3-fluoro-phenyl)-2-(chloromethyl)-3-(3-chlorophenyl)oxirane, anti-2-(3-fluorophenyl)-2-(chloro-methyl)-3-(4-fluorophenyl)oxirane, 2-(3-fluorophenyl)-2-(bromomethyl)-3-(2-methyl-phenyl)oxirane and 2-(3-fluorophenyl)-2-(CH₃O₂SO-methyl)-3-(2-methylphenyl)oxirane. B has in particular the meanings as specified herein for formula I, taking into account the compounds which are excluded. According to one aspect, B is unsubstituted phenyl or phenyl which comprises one, two or three substituents L selected from the group consisting of halogen, NO₂, amino, C₁-C₄-alkyl, C₁-C₄-alkoxy, C₁-C₄-haloalkyl, C₁-C₄-haloalkoxy, C₁-C₄-alkylamino, thio and C₁-C₄-alkylthio, except for the compounds mentioned.

According to a further aspect, B is not ortho-methylphenyl; according to a further aspect, B is not ortho-alkylphenyl.

One way of preparing compounds III.1 consists in converting the double bond in compounds of the formula IVa

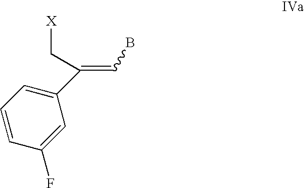

into the epoxide. X is as defined for formula III.1 and B is as defined for formula I. Epoxidation methods are known to the person skilled in the art. It is possible, for example, to use hydrogen peroxide/maleic anhydride for this purpose.

In formula IVa, the double bond can be present either in (E) or in (Z) configuration. This is indicated by the zig-zag bond between B and the double bond. The present invention furthermore provides compounds of the formula IVa in which B is as defined or as preferably defined for formula I, except for the compounds (Z)-1-[3-chloro-1-(2-chloro-phenyl)prop-1-en-2-yl]-3-fluorobenzene, (Z)-1-[3-chloro-1-(4-chlorophenyl)prop-1-en-2-yl]-3-fluorobenzene, (Z)-1-[3-chloro-1-(3-chlorophenyl)prop-1-en-2-yl]-3-fluoro-benzene and (Z)-1-[3-chloro-1-(4-fluorophenyl)prop-1-en-2-yl]-3-fluorobenzene. Here, X has the meanings as described above for formula III.1. According to one embodiment, B is not ortho-methylphenyl; according to a further aspect, B is not ortho-alkylphenyl.

Compounds IVa can be obtained from compounds IVc

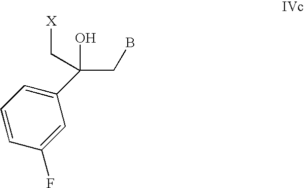

by reacting compounds IVc, for example, with acetic acid/H₂SO₄ in a suitable organic solvent, such as, for example, in ether, such as Et₂O or dioxane, to form the double bond. Suitable methods are known to the person skilled in the art. X is defined as for formula III and B is defined as for formula I.

Some compounds of the formula IVc are novel. Accordingly, the invention also provides compounds of the formula IVc, in which B is as defined or as preferably defined for formula I, except for the compounds 1-chloro-2-(3-fluorophenyl)-3-(2-chloro-phenyl)propan-2-ol, 1-chloro-2-(3-fluorophenyl)-3-(4-chlorophenyl)propan-2-ol, 1-chloro-2-(3-fluorophenyl)-3-(3-chlorophenyl)propan-2-ol and 1-chloro-2-(3-fluoro-phenyl)-3-(4-fluorophenyl)propan-2-ol. According to one embodiment, B is not ortho-methylphenyl; according to a further aspect, B is not ortho-alkylphenyl. X is as defined for formula III.1.

Compounds IVc can be obtained, for example, via a Grignard reaction according to the following scheme:

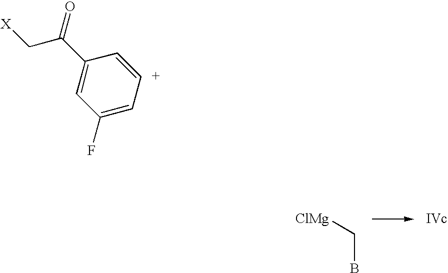

See also EP 409049.

Compounds of the formula III.1 can also be obtained from compounds of the formula III.2

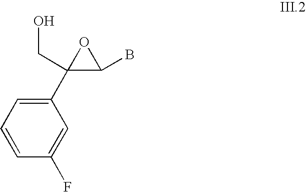

by introducing the leaving group X using methods known to the person skilled in the art. Thus, a compound of the formula III.2 is reacted, for example, with R—SO₂Y, where R is as defined for formula III.1 and Y is halogen, R—SO₂Y being, for example, mesyl chloride, in the presence of a base (for example NEt₃) (see also EP386557). To obtain compounds III.1 in which X is halogen, the corresponding compound III.2 can be reacted with C(Hal)₄ (Hal=Br or Cl) with PPh₃, for example in CH₂Cl₂, to give a compound III.1. Alternatively, a compound III.2 can be reacted with SOCl₂/pyridine (see also WO 2005/056548).

The present invention also provides compounds of the formula III.2 in which B is as defined or as preferably defined for formula I, except for the compound 2-hydroxy-methyl-2-(3-fluorophenyl)-3-(2-methylphenyl)oxirane. According to one embodiment, B is not ortho-methylphenyl; according to a further aspect, B is not ortho-alkylphenyl.

Compounds of the formula III.2 can be obtained from α,β-disubstituted acroleins of the type of the formula V

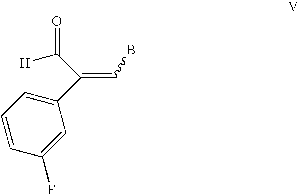

by initial epoxidation, for example with H₂O₂ in the presence of a base such as, for example, NaOH or by reaction with a peracid (for example MCPBA=m-chloroperoxy-benzoic acid) or tert-butyl hydroperoxide). This gives compounds of the formula Va

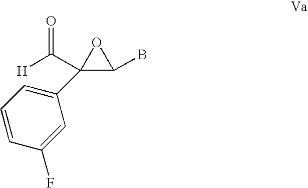

By reducing the aldehyde group in compounds Va, for example with NaBH₄ (see also EP 0 386 557A1), compounds III.2 are formed. Processes for epoxidation and reduction of the aldehyde group are well known to the person skilled in the art.

In formula V, the double bond can be present either in the (E) or in the (Z) configuration. This is indicated by the zig-zag bond between B and the double bond. Some of the compounds of the formula V are novel. Accordingly, the invention also provides compounds V in which B is as defined or as preferably defined for formula I, except for the compound (E)-2-(3-fluorophenyl)-3-(2-methylphenyl)propenal. According to one embodiment, B is not ortho-methylphenyl; according to a further aspect, B is not ortho-alkylphenyl.

Compounds Va in which B is defined or preferably defined as described herein for compounds of the formula I are also provided by the present invention. According to one embodiment, B is not ortho-methylphenyl; according to a further aspect, B is not ortho-alkylphenyl.

The compounds V can be synthesized, for example, analogously to the procedure described in DE3601927, i.e. by reacting compounds of the type of the formula VI

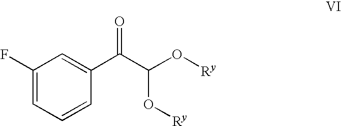

with appropriate phosphorus compounds of the Wittig or Horner-Emmons type with subsequent acidic cleavage of the acetal. In this case, R^{y} is in each case independently C₁-C₄-alkyl.

An alternative preparation of compounds V consists in the oxidation of compounds of the formula VII.

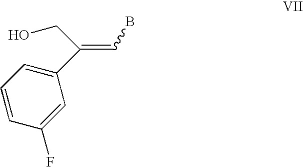

Suitable oxidizing agents and conditions are known to the person skilled in the art, for example a reaction according to Swern (Australian Journal of Chemistry, 57 (6), 537-548; 2004), reactions with hypervalent iodine compounds (Organic Letters, 5 (17), 2989-2992; 2003), with chromium compounds such as, for example, pyridinium dichromate (Tetrahedron, 45 (1), 239-58; 1989) or with manganese oxides such as, for example, MnO₂ (Journal of the American Chemical Society, 107 (13), 3963-71; 1985). The oxidation may also be carried out via a Dess-Martin oxidation in a solvent such as, for example, CH₂Cl₂.

In formula VII, the double bond may be present either in the (E) or in the (Z) configuration. This is indicated by the zig-zag bond between B and the double bond. Some compounds of the formula VII are novel. Accordingly, the present invention also relates to compounds of the formula VII in which B is as defined or preferred for formula I.

Starting with compounds VII, it is also possible to prepare compounds III.2 directly by epoxidation in the presence of a transition metal alkoxide such as, for example, V(O)(OR)₃ or Ti(OR)₄ (R=C₁-C₆-alkyl) and an oxidizing agent such as, for example, tBuOOH analogously to U.S. Pat. No. 5,399,708.

Compounds of the formula VII can be prepared from α,β-unsaturated acrylic esters of the formula VIII:

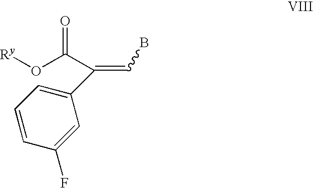

To this end, esters of the formula VIII are reduced to the alcohol VII. Suitable reducing procedures are well known to the person skilled in the art.

In formula VIII, the double bond may be present either in the (E) or in the (Z) configuration. This is indicated by the zig-zag bond between B and the double bond. Some compounds of the formula VIII are novel. Accordingly, the present invention also relates to compounds VIII in which B is as defined or preferred for formula I.

Compounds of the formula VIII can also be reduced in one step to the acrolein of the formula V, for example using metal hydrides such as, for example, diisobutylaluminum hydride at low temperatures. To this end, it is possible to use in particular aluminum hydrides, preferably lithium alanate (European Journal of Medicinal Chemistry, 40 (6), 529-541; 2005) or dialkylaluminum hydrides such as, for example, DIBAL-H (Synlett, (18), 3182-3184; 2006).

The acrylic esters of the formula VIII are obtainable from glyoxalic esters of the formula IX by reaction with phosphorus compounds, for example of the Horner-Emmons type or Wittig compounds.

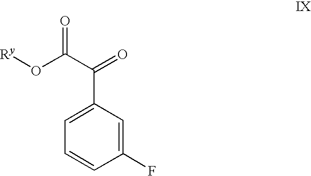

Such reactions are described, inter alia, Tetrahedron, 46 (13-14), 4951-94; 1990, Tetrahedron Letters, 47 (16), 2675-2678; 2006, Synthesis, (12), 1797-1802; 2003, WO9929645 or Synthetic Communications, 20 (12), 1781-91; 1990.

The synthesis of glyoxalic esters is described, inter alia, in Journal of Organic Chemistry, 52 (22), 5026-30; 1987 by reaction of Grignard compounds (starting with commercially available halogen compounds and magnesium) X¹MgA with oxalic esters of the formula

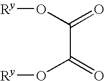

Suitable phosphorus compounds (of the Horner-Emmons type and Wittig type) can be prepared by known standard methods, for example from a compound of the type below:

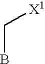

B is as defined above, X¹ is a leaving group such as, for example, a halide, preferably chlorine or bromine. The conversion of such halides into the desired Horner-Emmons or Wittig reagents can be carried out as described, for example, in Chemistry of Materials, 13 (9), 3009-3017; 2001, European Journal of Organic Chemistry, (7), 1247-1257; 2005 or WO1992/05145.

The alkyl halides are either commercially available or can be prepared by standard methods, for example by halogenation of the corresponding methyl compound. Suitable halogenating agents for this reaction are N-bromosuccinimide (Chemistry-A European Journal, 12 (21), 5632-5641; 2006) or N-chlorosuccinimide (Tetrahedron Letters, 47 (37), 6607-6609; 2006).

According to one alternative, compounds of the formula V can also be prepared via an aldol synthesis according to the scheme below:

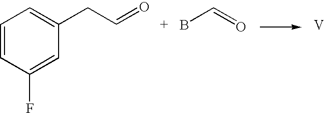

A further alternative of preparing compounds of the formula II consists in the epoxidation of a compound of the formula IVb.

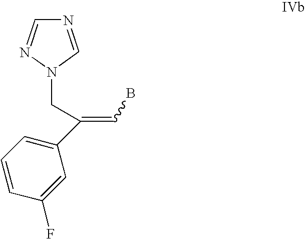

Suitable epoxidation methods are known to the person skilled in the art, see also the preparation of the compounds III.2 from compounds V.

In formula IVb, the double bond can be present either in the (E) or in the (Z) configuration. This is indicated by the zig-zag bond between B and the double bond. The present invention furthermore provides compounds of the formula IVb in which B is as defined or preferred for formula I. According to one embodiment, B is not ortho-methylphenyl; according to a further aspect, B is not ortho-alkylphenyl.

Compounds of the formula IVb can be obtained by reacting a compound of the formula IVa, as indicated above, in which X is a leaving group, as defined above, in particular a halide, with 1,2,4-triazole and a base. The reaction conditions can be chosen as described above for the preparation of compounds II starting from compounds III.

A further alternative of preparing compounds of the formula I consists in initially converting compounds of the formula III.1 (see above) with hydrazine into compounds of the formula IIIa.

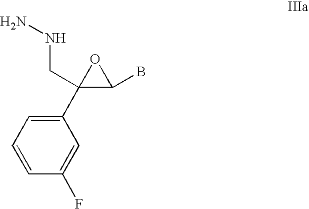

The present invention also provides compounds of the formula IIIa in which B is as defined or preferred for formula I.

Compounds of the formula IIIa can then be reacted with a thiocyanate YSCN in which Y is an alkali metal or ammonium, preferably sodium, potassium or ammonium, particularly preferably using NH₄SCN. This gives thiosemicarbazide compounds IIIb

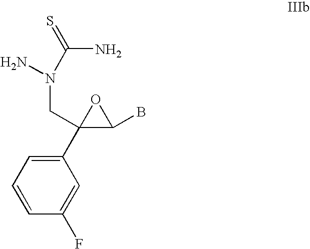

The triazolyl ring can be formed by reaction with formic acid, and what is obtained is the thione form of the corresponding compound of the formula I (D=SH) according to the invention, which, if appropriate, may be reacted further (see above). See also DE 19744400 (WO99/18088).

The present invention furthermore provides compounds of the formula IIIb in which B is as defined or preferred for formula I.

According to a further alternative, compounds IIIa can be reacted with formaldehyde ((CH₂O)ₙ) and a thiocyanate (YSCN, see above), which gives compounds of the formula IIIc

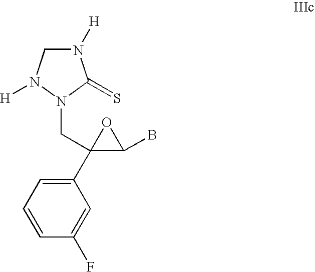

The triazolyl ring and thus the corresponding compound of the formula I is then formed by oxidation with, for example, iron(III) chloride in aqueous HCl (see also DE19961603 or WO 00/146158) or with oxygen in the presence of KOH and sulfur (see also WO 99/18087).

The present invention furthermore provides compounds of the formula IIIc in which B is as defined or preferred for formula I.

Yet another synthesis alternative consists in reacting a compound IIIa with a carbonyl compound (R^{x1}R^{x2})C═O (R^{x1}=C₁-C₄-alkyl or phenyl, R^{x2}=hydrogen or C₁-C₄-alkyl or R^{x1} and R^{x2} together form a —(CH₂)₅— chain), for example using acetone ((CH₃)₂CO) and a thiocyanate YSCN in which Y is sodium, potassium or ammonium, to give compounds IIId,

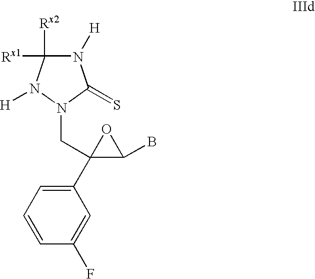

and then converting the compound formed in this manner with formic acid, if appropriate in the presence of a catalyst (for example HCl, H₂SO₄, p-toluenesulfonic acid, metal oxides such as, for example, amorphous TiO₂) into the corresponding triazole compound I. In this case, R^{x1} and R^{x2} are preferably both methyl (compounds IIId-1). See also DE19744401 and WO 99/18086.

The present invention furthermore provides compounds of the formula IIId in which B is as defined or preferred for formula I.

In some of the definitions of the symbols in the formulae given herein, collective terms are used which are generally representative of the following substituents:

halogen: fluorine, chlorine, bromine and iodine;

alkyl and the alkyl moieties of composite groups such as, for example, alkylamino: saturated straight-chain or branched hydrocarbon radicals having 1 to 4, 6, 8 or 12 carbon atoms, for example C₁-C₆-alkyl, such as methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-tri-methylpropyl, 1-ethyl-1-methylpropyl and 1-ethyl-2-methylpropyl;

haloalkyl: alkyl as mentioned above, where some or all of the hydrogen atoms in these groups are replaced by halogen atoms as mentioned above; in particular C₁-C₂-haloalkyl, such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl or 1,1,1-trifluoroprop-2-yl

alkenyl and also the alkenyl moieties in composite groups, such as alkenyloxy: unsaturated straight-chain or branched hydrocarbon radicals having 2 to 4, 2 to 6 or 2 to 8 carbon atoms and one double bond in any position. According to the invention, it may be preferred to use small alkenyl groups, such as (C₂-C₄)-alkenyl; on the other hand, it may also be preferred to employ larger alkenyl groups, such as (C₅-C₈)-alkenyl. Examples of alkenyl groups are, for example, C₂-C₆-alkenyl, such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl;

haloalkenyl: alkenyl as defined above, where some or all of the hydrogen atoms in these groups are replaced by halogen atoms as described above under haloalkyl, in particular by fluorine, chlorine or bromine;

alkadienyl: unsaturated straight-chain or branched hydrocarbon radicals having 4 to 6 or 4 to 8 carbon atoms and two double bonds in any position;

alkynyl and the alkynyl moieties in composite groups: straight-chain or branched hydrocarbon groups having 2 to 4, 2 to 6 or 2 to 8 carbon atoms and one or two triple bonds in any position, for example C₂-C₆-alkynyl, such as ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl and 1-ethyl-1-methyl-2-propynyl;

haloalkynyl: alkynyl as defined above, where some or all of the hydrogen atoms in these groups are replaced by halogen atoms as described above under haloalkyl, in particular by fluorine, chlorine or bromine;

cycloalkyl and also the cycloalkyl moieties in composite groups: mono- or bicyclic saturated hydrocarbon groups having 3 to 8, in particular 3 to 6, carbon ring members, for example C₃-C₆-cycloalkyl, such as cyclopropyl, cyclobutyl, cyclopentyl, cyclohexyl;

halocycloalkyl: cycloalkyl as defined above, where some or all of the hydrogen atoms in these groups are replaced by halogen atoms as described above under haloalkyl, in particular by fluorine, chlorine or bromine;

cycloalkenyl: monocyclic monounsaturated hydrocarbon groups having preferably 3 to 8 or 4 to 6, in particular 5 to 6, carbon ring members, such as cyclopenten-1-yl, cyclopenten-3-yl, cyclohexen-1-yl, cyclohexen-3-yl, cyclohexen-4-yl and the like;

halocycloalkenyl: cycloalkenyl as defined above, where some or all of the hydrogen atoms in these groups are replaced by halogen atoms as described above under haloalkyl, in particular by fluorine, chlorine or bromine;

alkoxy: an alkyl group as defined above which is attached via an oxygen, preferably having 1 to 8, more preferably 2 to 6, carbon atoms. Examples are: methoxy, ethoxy, n-propoxy, 1-methylethoxy, butoxy, 1-methylpropoxy, 2-methylpropoxy or 1,1-dimethylethoxy, and also for example, pentoxy, 1-methylbutoxy, 2-methylbutoxy, 3-methylbutoxy, 1,1-dimethylpropoxy, 1,2-dimethylpropoxy, 2,2-dimethylpropoxy, 1-ethylpropoxy, hexoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy or 1-ethyl-2-methylpropoxy;

haloalkoxy: alkoxy as defined above, where some or all of the hydrogen atoms in these groups are replaced by halogen atoms as described above under haloalkyl, in particular by fluorine, chlorine or bromine. Examples are OCH₂F, OCHF₂, OCF₃, OCH₂Cl, OCHCl₂, OCCl₃, chlorofluoromethoxy, dichlorofluoromethoxy, chlorodifluoro-methoxy, 2-fluoroethoxy, 2-chloroethoxy, 2-bromoethoxy, 2-iodoethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy, OC₂F₅, 2-fluoropropoxy, 3-fluoropropoxy, 2,2-difluoropropoxy, 2,3-difluoropropoxy, 2-chloropropoxy, 3-chloropropoxy, 2,3-dichloropropoxy, 2-bromopropoxy, 3-bromopropoxy, 3,3,3-trifluoropropoxy, 3,3,3-trichloropropoxy, OCH₂—C₂F₅, OCF₂—C₂F₅, 1-(CH₂F)-2-fluoroethoxy, 1-(CH₂Cl)-2-chloroethoxy, 1-(CH₂Br)-2-bromoethoxy, 4-fluorobutoxy, 4-chlorobutoxy, 4-bromobutoxy or nonafluorobutoxy; and also 5-fluoropentoxy, 5-chloropentoxy, 5-bromopentoxy, 5-iodopentoxy, undecafluoropentoxy, 6-fluorohexoxy, 6-chlorohexoxy, 6-bromohexoxy, 6-iodohexoxy or dodecafluorohexoxy.

alkylene: divalent unbranched chains of CH₂ groups. Preference is given to (C₁-C₆)-alkylene, more preference to (C₂-C₄)-alkylene; furthermore, it may be preferred to use (C₁-C₃)-alkylene groups. Examples of preferred alkylene radicals are CH₂, CH₂CH₂, CH₂CH₂CH₂, CH₂(CH₂)₂CH₂, CH₂(CH₂)₃CH₂ and CH₂(CH₂)₄CH₂;

a 3-, 4-, 5-, 6-, 7-, 8-, 9- or 10-membered saturated or partially unsaturated heterocycle which contains 1, 2, 3 or 4 heteroatoms from the group consisting of O, N and S, where the heterocycle in question may be attached via a carbon atom or, if present, via a nitrogen atom. According to the invention, it may be preferred for the heterocycle in question to be attached via carbon, on the other hand, it may also be preferred for the heterocycle to be attached via nitrogen. In particular:
a three- or four-membered saturated heterocycle (hereinbelow also referred to as heterocyclyl) which comprises one or two heteroatoms from the group consisting of O, N and S as ring members;
a five- or six-membered saturated or partially unsaturated heterocycle which comprises one, two, three or four heteroatoms from the group consisting of O, N and S as ring members: for example monocyclic saturated or partially unsaturated heterocycles which, in addition to carbon ring members, comprise one, two or three nitrogen atoms and/or one oxygen or sulfur atom or one or two oxygen and/or sulfur atoms, for example 2-tetrahydrofuranyl, 3-tetrahydrofuranyl, 2-tetrahydrothienyl, 3-tetrahydrothienyl, 2-pyrrolidinyl, 3-pyrrolidinyl, 3-isoxazolidinyl, 4-isoxazolidinyl, 5-isoxazolidinyl, 3-isothiazolidinyl, 4-isothiazolidinyl, 5-isothiazolidinyl, 3-pyrazolidinyl, 4-pyrazolidinyl, 5-pyrazolidinyl, 2-oxazolidinyl, 4-oxazolidinyl, 5-oxazolidinyl, 2-thiazolidinyl, 4-thiazolidinyl, 5-thiazolidinyl, 2-imidazolidinyl, 4-imidazolidinyl, 1,2,4-oxadiazolidin-3-yl, 1,2,4-oxadiazolidin-5-yl, 1,2,4-thiadiazolidin-3-yl, 1,2,4-thiadiazolidin-5-yl, 1,2,4-triazolidin-3-yl, 1,3,4-oxadiazolidin-2-yl, 1,3,4-thiadiazolidin-2-yl, 1,3,4-triazolidin-2-yl, 2,3-dihydrofur-2-yl, 2,3-dihydrofur-3-yl, 2,4-dihydrofur-2-yl, 2,4-dihydrofur-3-yl, 2,3-dihydrothien-2-yl, 2,3-dihydrothien-3-yl, 2,4-dihydrothien-2-yl, 2,4-dihydrothien-3-yl, 2-pyrrolin-2-yl, 2-pyrrolin-3-yl, 3-pyrrolin-2-yl, 3-pyrrolin-3-yl, 2-isoxazolin-3-yl, 3-isoxazolin-3-yl, 4-isoxazolin-3-yl, 2-isoxazolin-4-yl, 3-isoxazolin-4-yl, 4-isoxazolin-4-yl, 2-isoxazolin-5-yl, 3-isoxazolin-5-yl, 4-isoxazolin-5-yl, 2-isothiazolin-3-yl, 3-isothiazolin-3-yl, 4-isothiazolin-3-yl, 2-isothiazolin-4-yl, 3-isothiazolin-4-yl, 4-isothiazolin-4-yl, 2-isothiazolin-5-yl, 3-isothiazolin-5-yl, 4-isothiazolin-5-yl, 2,3-dihydropyrazol-1-yl, 2,3-dihydropyrazol-2-yl, 2,3-dihydropyrazol-3-yl, 2,3-dihydropyrazol-4-yl, 2,3-dihydropyrazol-5-yl, 3,4-dihydropyrazol-1-yl, 3,4-dihydropyrazol-3-yl, 3,4-dihydropyrazol-4-yl, 3,4-dihydropyrazol-5-yl, 4,5-dihydropyrazol-1-yl, 4,5-dihydropyrazol-3-yl, 4,5-dihydropyrazol-4-yl, 4,5-dihydropyrazol-5-yl, 2,3-dihydrooxazol-2-yl, 2,3-dihydrooxazol-3-yl, 2,3-dihydrooxazol-4-yl, 2,3-dihydrooxazol-5-yl, 3,4-dihydrooxazol-2-yl, 3,4-dihydrooxazol-3-yl, 3,4-dihydrooxazol-4-yl, 3,4-dihydro-oxazol-5-yl, 3,4-dihydrooxazol-2-yl, 3,4-dihydrooxazol-3-yl, 3,4-dihydrooxazol-4-yl, 2-piperidinyl, 3-piperidinyl, 4-piperidinyl, 1,3-dioxan-5-yl, 2-tetrahydropyranyl, 4-tetrahydropyranyl, 2-tetrahydrothienyl, 3-hexahydropyridazinyl, 4-hexa-hydropyridazinyl, 2-hexahydropyrimidinyl, 4-hexahydropyrimidinyl, 5-hexahydro-pyrimidinyl, 2-piperazinyl, 1,3,5-hexahydrotriazin-2-yl and 1,2,4-hexahydrotriazin-3-yl and also the corresponding -ylidene radicals;
a seven-membered saturated or partially unsaturated heterocycle which comprises one, two, three or four heteroatoms from the group consisting of O, N and S as ring members: for example mono- and bicyclic heterocycles having 7 ring members which, in addition to carbon ring members, comprise one, two or three nitrogen atoms and/or one oxygen or sulfur atom or one or two oxygen and/or sulfur atoms, for example tetra- and hexahydroazepinyl, such as 2,3,4,5-tetrahydro[1H]azepin-1-, -2-, -3-, -4-, -5-, -6- or -7-yl, 3,4,5,6-tetra-hydro[2H]azepin-2-, -3-, -4-, -5-, -6- or -7-yl, 2,3,4,7-tetrahydro[1H]azepin-1-, -2-, -3-, -4-, -5-, -6- or -7-yl, 2,3,6,7-tetrahydro[1H]azepin-1-, -2-, -3-, -4-, -5-, -6- or -7-yl, hexahydroazepin-1-, -2-, -3- or -4-yl, tetra- and hexahydrooxepinyl such as 2,3,4,5-tetrahydro[1H]oxepin-2-, -3-, -4-, -5-, -6- or -7-yl, 2,3,4,7-tetrahydro[1H]oxepin-2-, -3-, -4-, -5-, -6- or -7-yl, 2,3,6,7-tetrahydro[1H]oxepin-2-, -3-, -4-, -5-, -6- or -7-yl, hexahydroazepin-1-, -2-, -3- or -4-yl, tetra- and hexahydro-1,3-diazepinyl, tetra- and hexahydro-1,4-diazepinyl, tetra- and hexahydro-1,3-oxazepinyl, tetra- and hexahydro-1,4-oxazepinyl, tetra- and hexahydro-1,3-dioxepinyl, tetra- and hexahydro-1,4-dioxepinyl and the corresponding ylidene radicals;

a 5-, 6-, 7-, 8-, 9- or 10-membered aromatic heterocycle which comprises 1, 2, 3 or 4 heteroatoms from the group consisting of O, N and S: in particular a five- or six-membered aromatic mono- or bicyclic heterocycle which comprises one, two, three or four heteroatoms from the group consisting of O, N and S: the heterocycle in question may be attached via a carbon atom or, if present, via a nitrogen atom. According to the invention, it may be preferred for the heterocycle in question to be attached via carbon, on the other hand, it may also be preferred for the heterocycle to be attached via nitrogen. The heterocycle is in particular:
5-membered heteroaryl which comprises one, two, three or four nitrogen atoms or one, two or three nitrogen atoms and/or one sulfur or oxygen atom, where the heteroaryl may be attached via carbon or nitrogen, if present: 5-membered heteroaryl groups which, in addition to carbon atoms, may comprise one to four nitrogen atoms or one, two or three nitrogen atoms and/or one sulfur or oxygen atom as ring members, for example furyl, thienyl, pyrrolyl, pyrazolyl, imidazolyl, triazolyl (1,2,3-; 1,2,4-triazolyl), tetrazolyl, oxazolyl, isoxazolyl, 1,3,4-oxadiazolyl, thiazolyl, isothiazolyl and thiadiazolyl, in particular 2-furyl, 3-furyl, 2-thienyl, 3-thienyl, 2-pyrrolyl, 3-pyrrolyl, 3-isoxazolyl, 4-isoxazolyl, 5-isoxazolyl, 3-isothiazolyl, 4-isothiazolyl, 5-isothiazolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 2-imidazolyl, 4-imidazolyl, 1,2,4-oxadiazol-3-yl, 1,2,4-oxadiazol-5-yl, 1,2,4-thiadiazol-3-yl, 1,2,4-thiadiazol-5-yl, 1,2,4-triazol-3-yl, 1,3,4-oxadiazol-2-yl, 1,3,4-thiadiazol-2-yl and 1,3,4-triazol-2-yl;
6-membered heteroaryl which comprises one, two, three or four, preferably one, two or three, nitrogen atoms, where the heteroaryl may be attached via carbon or nitrogen, if present: 6-ring heteroaryl groups which, in addition to carbon atoms, may comprise one to four or one, two or three nitrogen atoms as ring members, for example pyridinyl, pyrimidinyl, pyrazinyl, pyridazinyl, 1,2,3-triazinyl, 1,2,4-triazinyl, 1,3,5-triazinyl, in particular 2-pyridinyl, 3-pyridinyl, 4-pyridinyl, 3-pyridazinyl, 4-pyridazinyl, 2-pyrimidinyl, 4-pyrimidinyl, 5-pyrimidinyl, 2-pyrazinyl, 1,3,5-triazin-2-yl and 1,2,4-triazin-3-yl.

The novel compounds according to the invention comprise chiral centers and are generally obtained in the form of racemates or as diastereomer mixtures of erythro and threo forms. The erythro and threo diastereomers of the compounds according to the invention can be separated and isolated in pure form, for example, on the basis of their different solubilities or by column chromatography. Using known methods, such uniform pairs of diastereomers can be used to obtain uniform enantiomers. Suitable for use as antimicrobial agents are both the uniform diastereomers or enantiomers and mixtures thereof obtained in the synthesis. This applies correspondingly to the fungicidal compositions.

Accordingly, the invention provides both the pure enantiomers or diastereomers and mixtures thereof. This applies to the compounds according to the invention of the formula I and, if appropriate, correspondingly to their precursors. The scope of the present invention includes in particular the (R) and (S) isomers and the racemates of the compounds according to the invention, in particular of the formula I or II, which have centers of chirality. Suitable compounds according to the invention, in particular of the formula I or II, also include all possible stereoisomers (cis/trans isomers) and mixtures thereof.

The compounds according to the invention, in particular of the formula I or II, may be present in various crystal modifications whose biological activity may be different. They are included in the scope of the present invention.

In the compounds according to the invention I, particular preference is given to the following meanings of the substituents, in each case on their own or in combination.

According to one embodiment of the invention, B is unsubstituted phenyl.

According to a further embodiment, B is phenyl which comprises one, two, three or four independently selected substituents L.

According to a further embodiment, the phenyl ring is monosubstituted by a substituent L, where L according to a specific aspect of this embodiment is located in the ortho-position to the point of attachment of the phenyl ring to the oxirane ring.

According to a further embodiment, B is phenyl which comprises two or three independently selected substituents L.

According to a further embodiment of the invention, B is a phenyl ring which comprises a substituent L in the ortho-position and furthermore a further independently selected substituent L. According to one aspect, the phenyl ring is 2,3-disubstituted. According to a further aspect, the phenyl ring is 2,4-disubstituted. According to yet a further aspect, the phenyl ring is 2,5-disubstituted. According to yet a further aspect, the phenyl ring is 2,6-disubstituted.

According to a further embodiment of the invention, B is a phenyl ring which comprises a substituent L in the ortho-position and furthermore two further independently selected substituents L. According to one aspect, the phenyl ring is 2,3,5-trisubstituted.

According to a further aspect, the phenyl ring is 2,3,4-trisubstituted. According to yet a further aspect, the phenyl ring is 2,4,5-trisubstituted.

L independently has the meanings mentioned above for L. Unless indicated otherwise, L is preferably independently selected from the list consisting of halogen, cyano, nitro, cyanato (OCN), C₁-C₄-haloalkyl, C₁-C₄-alkoxy, C₁-C₄-haloalkoxy, C₃-C₆-cycloalkyl, C₃-C₆-halocycloalkyl, S-A¹, C(═O)A², C(═S)A², NA³A⁴; where A¹, A², A³, A⁴ are as defined below:
A¹ is hydrogen, hydroxyl, C₁-C₄-alkyl, C₁-C₄-haloalkyl;
A² is one of the groups mentioned for A¹ or C₁-C₄-alkoxy, C₁-C₄-haloalkoxy, C₃-C₆-cycloalkyl, C₃-C₆-halocycloalkyl, C₃-C₆-cycloalkoxy or C₃-C₆-halocycloalkoxy;
A³, A⁴ independently of one another are hydrogen, C₁-C₄-alkyl, C₁-C₄-haloalkyl;
where the aliphatic and/or alicyclic groups of the radical definitions of L for their part may carry one, two, three or four identical or different groups R^{L}:
R^{L} is halogen, cyano, nitro, C₁-C₄-alkyl, C₁-C₄-haloalkyl, C₁-C₄-alkoxy, C₁-C₄-haloalkoxy, C₃-C₆-cycloalkyl, C₃-C₆-halocycloalkyl, amino, C₁-C₈-alkylamino, di-C₁-C₈-alkylamino.

Furthermore preferably, L is independently selected from the group consisting of halogen, NO₂, amino, C₁-C₄-alkyl, C₁-C₄-alkoxy, C₁-C₄-haloalkyl, C₁-C₄-haloalkoxy, C₁-C₄-alkylamino, C₁-C₄-dialkylamino, thio and C₁-C₄-alkylthio.

Furthermore preferably, L is independently selected from the group consisting of halogen, C₁-C₄-alkyl, C₁-C₄-haloalkyl, C₁-C₄-alkoxy, C₁-C₄-haloalkoxy and C₁-C₄-haloalkylthio.

According to a further preferred embodiment, L is independently selected from the group consisting of F, Cl, Br, CH₃, C₂H₅, i-C₃H₇, t-C₄H₉, OCH₃, OC₂H₅, CF₃, CCl₃, CHF₂, CClF₂, OCF₃, OCHF₂ and SCF₃, in particular selected from the group consisting of F, Cl, CH₃, C₂H₅, OCH₃, OC₂H₅, CF₃, CHF₂, OCF₃, OCHF₂ and SCF₃. According to one aspect, L is independently selected from the group consisting of F, Cl, CH₃, OCH₃, CF₃, OCF₃ and OCHF₂. It may be preferred for L to be independently F or Cl.

In a further embodiment, the substituent B is phenyl which is substituted by one, two or three halogen atoms.

In a further embodiment, B is phenyl which is unsubstituted or substituted by one, two or three substituents independently of one another selected from the group consisting of halogen, NO₂, amino, C₁-C₈-alkyl, C₁-C₄-alkoxy, C₁-C₄-haloalkyl, C₁-C₄-haloalkoxy, C₁-C₄-alkylamino, thio and C₁-C₄-alkylthio.

In a further embodiment of the invention, B is not ortho-methylphenyl.

The meanings of the variables B and L described above for compounds I apply correspondingly to the precursors of the compounds according to the invention.

According to one embodiment of the invention, D is a group SR, where R is hydrogen (compounds I-1). According to a further embodiment, D is a group SR, where R is C₁-C₄-alkyl, in particular methyl or ethyl, preferably methyl.

According to a further embodiment of the invention, D is a group SR where R is C(═O)R³ and R³ is NA³A⁴, where A³ and A⁴ independently of one another are hydrogen or C₁-C₈-alkyl.

According to a further embodiment of the invention, D is a group SR where R is C(═O)R³ and R³ is hydrogen, C₁-C₄-alkyl, C₁-C₄-alkoxy, C₁-C₄-haloalkoxy, phenyl or benzyl. According to a specific aspect thereof, R³ is hydrogen. According to a further aspect thereof, R³ is C₁-C₄-alkyl, in particular methyl or ethyl, preferably methyl. According to yet a further aspect, R³ is C₁-C₄-haloalkyl, in particular trifluoromethyl. According to yet a further aspect, R³ is C₁-C₄-alkoxy, in particular methoxy or ethoxy.

According to a further embodiment of the invention, D is a group SR where R is C(═O)R³ and R³ is (C₁-C₄)-alkylamino, di-(C₁-C₄)-alkylamino or phenylamino. According to one aspect thereof, R³ is methylamino, dimethylamino, ethylamino, diethylamino or phenylamino.

According to a further embodiment of the invention, D is a group SR where R is CN.

According to a further embodiment of the invention, D is a group SR where R is SO₂R⁴ and R⁴ is C₁-C₄-alkyl, phenyl-C₁-C₄-alkyl or phenyl, where the phenyl groups are in each case unsubstituted or substituted by one, two or three groups independently selected from the group consisting of halogen and C₁-C₄-alkyl.

According to a further embodiment of the invention, D is a group SM where M is an alkali metal cation, an equivalent of an alkaline earth metal cation, an equivalent of a copper, zinc, iron or nickel cation or an ammonium cation of the formula (E)

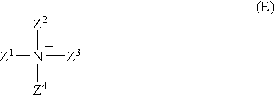

in which

Z¹ and Z² independently are hydrogen or C₁-C₄-alkyl; and

Z³ and Z⁴ independently are hydrogen, C₁-C₄-alkyl, benzyl or phenyl.

According to one embodiment, M is Na, ½ Cu, ⅓ Fe, HN(CH₃)₃, HN(C₂H₅)₃, N(CH₃)₄ or H₂N(C₃H₇)₂, in particular Na, ½ Cu, HN(CH₃)₃ or HN(C₂H₅)₃, especially Na, ½ Cu, HN(CH₃)₃ or HN(C₂H₅)₃.

According to a further embodiment of the invention, the invention relates to compounds of the formula I, wherein the variables have the following meanings:
B is phenyl, which is substituted in the ortho-position by a further substituent L, where L is:
   L fluorine, chlorine, bromine, C₁-C₈-alkyl, C₁-C₈-alkoxy, C₃-C₈-cycloalkyl, C₃-C₈-cycloalkoxy, C₁-C₈-alkylcarbonyloxy, C(═O)A², where A² is:
      A² hydrogen, hydroxyl, C₁-C₈-alkyl, C₁-C₈-haloalkyl, amino, C₁-C₈-alkylamino or di-C₁-C₈-alkylamino, C₁-C₈-alkoxy;
         D —S—R, where
   R is hydrogen, C1-C8-alkyl, C1-C8-haloalkyl, C2-C8-alkenyl, C2-C8-haloalkenyl, C2-C8-alkynyl, C2-C8-halogenalkynyl, C(═O)R3, C(═S)R3, SO₂R4 or CN; where
   R3 is hydrogen, C1-C8-alkyl, C1-C8-haloalkyl, C1-C8-alkoxy, C1-C8-haloalkoxy or NA3A4; and
   R4 is C1-C8-alkyl, phenyl-C1-C8-alkyl or phenyl, where the phenyl groups are in each case unsubstituted or substituted by one, two or three groups independently selected from halogen and C1-C4-alkyl;
      a group DI

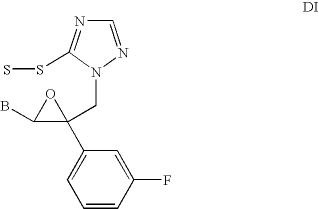

where B is as defined above;
   a group DII

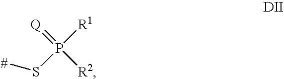

where # is the point of attachment to the triazolyl ring and Q, R¹ and R² are:
Q O or S;
R¹, R² independently of one another C₁-C₈-alkyl, C₁-C₈-haloalkyl, C₁-C₈-alkoxy, C₁-C₈-alkoxy-C₁-C₈-alkoxy, C₁-C₈-haloalkoxy, C₁-C₈-alkylthio, C₂-C₈-alkenylthio, C₂-C₈-alkynyl-thio, C₃-C₈-cycloalkyl, C₃-C₈-cycloalkylthio, phenyl, phenyl-C₁-C₄-alkyl, phenoxy, phenylthio, phenyl-C₁-C₄-alkoxy or NR⁵R⁶, where R⁵ is H or C₁-C₈-alkyl and R⁶ is C₁-C₈-alkyl, phenyl-C₁-C₄-alkyl or phenyl or R⁵ and R⁶ together are an alkylene chain having four or five C atoms or form a radical of the formula —CH₂—CH₂—O—CH₂—CH₂— or —CH₂—CH₂—NR⁷—CH₂—CH₂—, wherein R⁷ is hydrogen or C₁-C₄-alkyl; where the aromatic groups in the aforementioned radicals are in each case independently of one another unsubstituted or substituted by one, two or three groups selected from halogen and C₁-C₄-alkyl;
or
   a group SM, where M is:
M an alkali metal cation, an equivalent of an alkaline earth metal cation, an equivalent of a copper, zinc, iron or nickel cation or an ammonium cation of the formula (E)

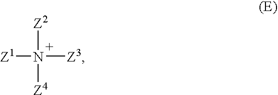

wherein
   Z¹ and Z² are independently hydrogen or C₁-C₈-alkyl;
   Z³ and Z⁴ are independently hydrogen, C₁-C₈-alkyl, benzyl or phenyl; where the phenyl groups are in each case unsubstituted or are substituted by one, two or three groups independently selected from halogen and C₁-C₄-alkyl;
and their agriculturally acceptable salts.

According to a further embodiment of the invention, the invention relates to compounds of the formula I wherein the variables have the following meanings:
B is phenyl, which is substituted in the ortho-position by a further substituent L, where L is:
   L fluorine, chlorine, bromine, methyl;
      D —S—R, where
   R is hydrogen, C₁-C₈-alkyl, C(═O)R3, or CN; where
   R3 is C1-C8-alkyl;
   SO₂R4, where
      R4 is C1-C8-alkyl;
   a group DI

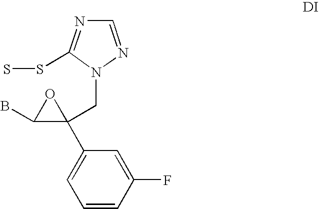

where B is as defined above; or
a group SM, where M is:
   M an alkali metal cation, an equivalent of an alkaline earth metal cation, an equivalent of a copper, zinc, iron or nickel cation or an ammonium cation of the formula (E)

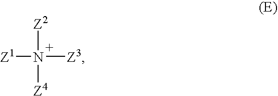

wherein
   Z¹ and Z² independently are hydrogen or C₁-C₈-alkyl;
   Z³ and Z⁴ independently are hydrogen, C₁-C₈-alkyl, benzyl or phenyl; where the phenyl groups are in each case unsubstituted or are substituted by one, two or three groups independently selected from halogen and C₁-C₄-alkyl;
and their agriculturally acceptable salts.

According to a further embodiment of the invention, D in the compounds of the formula I represents —SO₂R⁴, where R⁴ and B are as defined herein.

According to a further embodiment of the invention, D is a group DI (compounds I-2) where B is independently as defined or preferred herein:

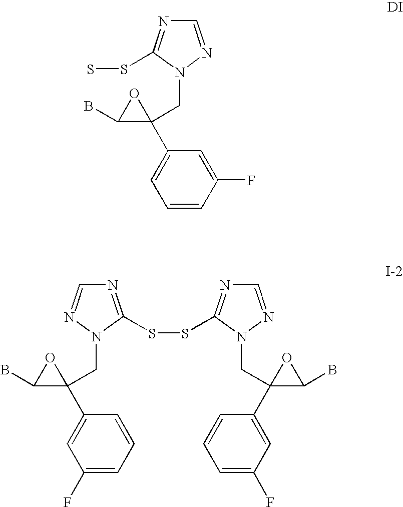

Preferably, both Bs in the compounds I-2 have the same meaning.

According to a further embodiment of the invention, D is a group DII where # is the point of attachment to the triazolyl ring and Q, R¹ and R² are as defined or preferred herein:

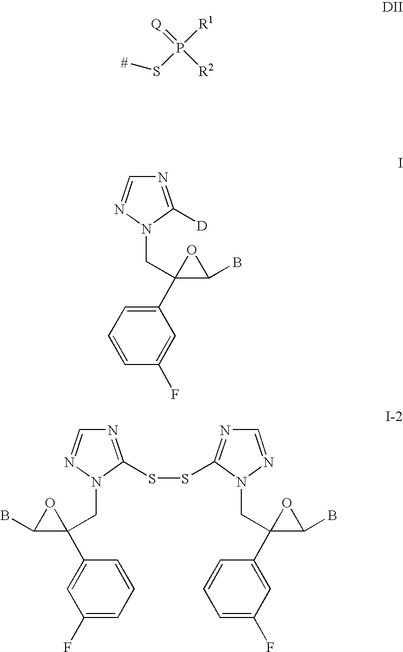

In particular with a view to their use, preference is given to the compounds I according to the invention compiled in Tables 1a to 10a below. The groups mentioned for a substituent in the tables are furthermore per se, independently of the combination in which they are mentioned, a particularly preferred aspect of the substituent in question.

### Table 1a

Compounds I in which D is SH and B corresponds in each case to one row of Table A (compounds I.1aA-1 to I.1aA-255)

### Table 2a

Compounds I in which D is S—CH₃ and B corresponds in each case to one row of Table A (compounds I.2aA-1 to I.2aA-255)

### Table 3a

Compounds I in which D is S—C₂H₅ and B corresponds in each case to one row of Table A (compounds I.3aA-1 to I.3aA-255)

### Table 4a

Compounds I in which D is SM where M is Na, and B corresponds in each case to one row of Table A (compounds I.4aA-1 to I.4aA-255)

### Table 5a

Compounds I in which D is SM, where M is ½ Cu, and B corresponds in each case to one row of Table A (compounds I.5aA-1 to I.5aA-255)

### Table 6a

Compounds I in which D is SM, where M is NHEt₃, and B corresponds in each case to one row of Table A (compounds I.6aA-1 to I.6aA-255)

### Table 7a

Compounds I in which D is S—CN and B corresponds in each case to one row of Table A (compounds I.7aA-1 to I.7aA-255)

### Table 8a

Compounds I in which D is S—C(═O)CH₃ and B corresponds in each case to one row of Table A (compounds I.8aA-1 to I.8aA-255)

### Table 9a

Compounds I in which D is S—C(═O)OCH₃ and B corresponds in each case to one row of Table A (compounds I.9aA-1 to I.9aA-255)

### Table 10a

Compounds I-2 in which both variables B correspond in each case to one row of Table A (compounds I-2.10aA-1 to I-2.10aA-255)

TABLE A

| Row | B |
|---|---|
| A-1 | phenyl |
| A-2 | 2-chlorophenyl |
| A-3 | 3-chlorophenyl |
| A-4 | 4-chlorophenyl |
| A-5 | 2-fluorophenyl |
| A-6 | 3-fluorophenyl |
| A-7 | 4-fluorophenyl |
| A-8 | 2-methylphenyl |
| A-9 | 3-methylphenyl |
| A-10 | 4-methylphenyl |
| A-11 | 2-ethylphenyl |
| A-12 | 3-ethylphenyl |
| A-13 | 4-ethylphenyl |
| A-14 | 2-methoxyphenyl |
| A-15 | 3-methoxyphenyl |
| A-16 | 4-methoxyphenyl |
| A-17 | 2-trifluoromethylphenyl |
| A-18 | 3-trifluoromethylphenyl |
| A-19 | 4-trifluoromethylphenyl |
| A-20 | 2-trifluoromethoxyphenyl |
| A-21 | 3-trifluoromethoxyphenyl |
| A-22 | 4-trifluoromethoxyphenyl |
| A-23 | 2-difluoromethoxyphenyl |
| A-24 | 3-difluoromethoxyphenyl |
| A-25 | 4-difluoromethoxyphenyl |
| A-26 | 2-trifluoromethylthiophenyl |
| A-27 | 3-trifluoromethylthiophenyl |
| A-28 | 4-trifluoromethylthiophenyl |
| A-29 | 2,3-dichlorophenyl |
| A-30 | 2,4-dichlorophenyl |
| A-31 | 2,5-dichlorophenyl |
| A-32 | 2,6-dichlorophenyl |
| A-33 | 3,4-dichlorophenyl |
| A-34 | 3,5-dichlorophenyl |
| A-35 | 2,3-difluorophenyl |
| A-36 | 2,4-difluorophenyl |
| A-37 | 2,5-difluorophenyl |
| A-38 | 2,6-difluorophenyl |
| A-39 | 3,4-difluorophenyl |
| A-40 | 3,5-difluorophenyl |
| A-41 | 2,3-dimethylphenyl |
| A-42 | 2,4-dimethylphenyl |
| A-43 | 2,5-dimethylphenyl |
| A-44 | 2,6-dimethylphenyl |
| A-45 | 3,4-dimethylphenyl |
| A-46 | 3,5-dimethylphenyl |
| A-47 | 2,3-diethylphenyl |
| A-48 | 2,4-diethylphenyl |
| A-49 | 2,5-diethylphenyl |
| A-50 | 2,6-diethylphenyl |
| A-51 | 3,5-diethylphenyl |
| A-52 | 3,4-diethylphenyl |
| A-53 | 2,3-dimethoxyphenyl |
| A-54 | 2,4-dimethoxyphenyl |
| A-55 | 2,5-dimethoxyphenyl |
| A-56 | 2,6-dimethoxyphenyl |
| A-57 | 3,4-dimethoxyphenyl |
| A-58 | 3,5-dimethoxyphenyl |
| A-59 | 2,3-di(trifluoromethyl)phenyl |
| A-60 | 2,4-di(trifluoromethyl)phenyl |
| A-61 | 2,5-di(trifluoromethyl)phenyl |
| A-62 | 2,6-di(trifluoromethyl)phenyl |
| A-63 | 3,4-di(trifluoromethyl)phenyl |
| A-64 | 3,5-di(trifluoromethyl)phenyl |
| A-65 | 2,3-di(trifluoromethoxy)phenyl |
| A-66 | 2,4-di(trifluoromethoxy)phenyl |
| A-67 | 2,5-di(trifluoromethoxy)phenyl |
| A-68 | 2,6-di(trifluoromethoxy)phenyl |
| A-69 | 3,4-di(trifluoromethoxy)phenyl |
| A-70 | 3,5-di(trifluoromethoxy)phenyl |
| A-71 | 2,3-di(difluoromethoxy)phenyl |
| A-72 | 2,4-di(difluoromethoxy)phenyl |
| A-73 | 2,5-di(difluoromethoxy)phenyl |
| A-74 | 2,6-di(difluoromethoxy)phenyl |
| A-75 | 3,4-di(difluoromethoxy)phenyl |
| A-76 | 3,5-di(difluoromethoxy)phenyl |
| A-77 | 2,3-di(trifluoromethylthio)phenyl |
| A-78 | 2,4-di(trifluoromethylthio)phenyl |
| A-79 | 2,5-di(trifluoromethylthio)phenyl |
| A-80 | 2,6-di(trifluoromethylthio)phenyl |
| A-81 | 3,4-di(trifluoromethylthio)phenyl |
| A-82 | 3,5-di(trifluoromethylthio)phenyl |
| A-83 | 2-fluoro-3-chlorophenyl |
| A-84 | 2-fluoro-4-chlorophenyl |
| A-85 | 2-fluoro-5-chlorophenyl |
| A-86 | 2-fluoro-6-chlorophenyl |
| A-87 | 3-fluoro-4-chlorophenyl |
| A-88 | 3-fluoro-5-chlorophenyl |
| A-89 | 2-chloro-3-fluorophenyl |
| A-90 | 2-chloro-4-fluorophenyl |
| A-91 | 2-chloro-5-fluorophenyl |
| A-92 | 3-chloro-4-fluorophenyl |
| A-93 | 2-methyl-3-chlorophenyl |
| A-94 | 2-methyl-4-chlorophenyl |
| A-95 | 2-methyl-5-chlorophenyl |
| A-96 | 2-methyl-6-chlorophenyl |
| A-97 | 3-methyl-4-chlorophenyl |
| A-98 | 3-methyl-5-chlorophenyl |
| A-99 | 2-chloro-3-methylphenyl |
| A-100 | 2-chloro-4-methylphenyl |
| A-101 | 2-chloro-5-methylphenyl |
| A-102 | 3-chloro-4-methylphenyl |
| A-103 | 2-methyl-3-fluorophenyl |
| A-104 | 2-methyl-4-fluorophenyl |
| A-105 | 2-methyl-5-fluorophenyl |
| A-106 | 2-methyl-6-fluorophenyl |
| A-107 | 3-methyl-4-fluorophenyl |
| A-108 | 3-methyl-5-fluorophenyl |
| A-109 | 2-fluoro-3-methylphenyl |
| A-110 | 2-fluoro-4-methylphenyl |
| A-111 | 2-fluoro-5-methylphenyl |
| A-112 | 3-fluoro-4-methylphenyl |
| A-113 | 2-chloro-3-ethylphenyl |
| A-114 | 2-chloro-4-ethylphenyl |
| A-115 | 2-chloro-5-ethylphenyl |
| A-116 | 3-chloro-4-ethylphenyl |
| A-117 | 2-ethyl-3-chlorophenyl |
| A-118 | 2-ethyl-4-chlorophenyl |
| A-119 | 2-ethyl-5-chlorophenyl |
| A-120 | 2-ethyl-6-chlorophenyl |
| A-121 | 2-ethyl-3-fluorophenyl |
| A-122 | 2-ethyl-4-fluorophenyl |
| A-123 | 2-ethyl-5-fluorophenyl |
| A-124 | 2-ethyl-6-fluorophenyl |
| A-125 | 3-ethyl-4-fluorophenyl |
| A-126 | 3-ethyl-5-fluorophenyl |
| A-127 | 2-fluoro-3-ethylphenyl |
| A-128 | 2-fluoro-4-ethylphenyl |
| A-129 | 2-fluoro-5-ethylphenyl |
| A-130 | 3-fluoro-4-ethylphenyl |
| A-131 | 2-methoxy-3-chlorophenyl |
| A-132 | 2-methoxy-4-chlorophenyl |
| A-133 | 2-methoxy-5-chlorophenyl |
| A-134 | 2-methoxy-6-chlorophenyl |
| A-135 | 3-methoxy-4-chlorophenyl |
| A-136 | 3-methoxy-5-chlorophenyl |
| A-137 | 2-chloro-3-methoxyphenyl |
| A-138 | 2-chloro-4-methoxyphenyl |
| A-139 | 2-chloro-5-methoxyphenyl |
| A-140 | 3-chloro-4-methoxyphenyl |
| A-141 | 2-methoxy-3-fluorophenyl |
| A-142 | 2-methoxy-4-fluorophenyl |
| A-143 | 2-methoxy-5-fluorophenyl |
| A-144 | 2-methoxy-6-fluorophenyl |
| A-145 | 3-methoxy-4-fluorophenyl |
| A-146 | 3-methoxy-5-fluorophenyl |
| A-147 | 2-fluoro-3-methoxyphenyl |
| A-148 | 2-fluoro-4-methoxyphenyl |
| A-149 | 2-fluoro-5-methoxyphenyl |
| A-150 | 3-fluoro-4-methoxyphenyl |
| A-151 | 3-fluoro-5-methoxyphenyl |
| A-152 | 2-(trifluoromethyl)-3-chlorophenyl |
| A-153 | 2-(trifluoromethyl)-4-chlorophenyl |
| A-154 | 2-(trifluoromethyl)-5-chlorophenyl |
| A-155 | 2-(trifluoromethyl)-6-chlorophenyl |
| A-156 | 3-(trifluoromethyl)-4-chlorophenyl |
| A-157 | 3-(trifluoromethyl)-5-chlorophenyl |
| A-158 | 2-chloro-3-(trifluoromethyl)phenyl |
| A-159 | 2-chloro-4-(trifluoromethyl)phenyl |
| A-160 | 2-chloro-5-(trifluoromethyl)phenyl |
| A-161 | 3-chloro-4-(trifluoromethyl)phenyl |
| A-162 | 2-(trifluoromethyl)-3-fluorophenyl |
| A-163 | 2-(trifluoromethyl)-4-fluorophenyl |
| A-164 | 2-(trifluoromethyl)-5-fluorophenyl |
| A-165 | 2-(trifluoromethyl)-6-fluorophenyl |
| A-166 | 3-(trifluoromethyl)-4-fluorophenyl |
| A-167 | 3-(trifluoromethyl)-5-fluorophenyl |
| A-168 | 2-fluoro-3-(trifluoromethyl)phenyl |
| A-169 | 2-fluoro-4-(trifluoromethyl)phenyl |
| A-170 | 2-fluoro-5-(trifluoromethyl)phenyl |
| A-171 | 3-fluoro-4-(trifluoromethyl)phenyl |
| A-172 | 2-(trifluoromethoxy)-3-chlorophenyl |
| A-173 | 2-(trifluoromethoxy)-4-chlorophenyl |
| A-174 | 2-(trifluoromethoxy)-5-chlorophenyl |
| A-175 | 2-(trifluoromethoxy)-6-chlorophenyl |
| A-176 | 3-(trifluoromethoxy)-4-chlorophenyl |
| A-177 | 3-(trifluoromethoxy)-5-chlorophenyl |
| A-178 | 2-chloro-3-(trifluoromethoxy)phenyl |
| A-179 | 2-chloro-4-(trifluoromethoxy)phenyl |
| A-180 | 2-chloro-5-(trifluoromethoxy)phenyl |
| A-181 | 3-chloro-4-(trifluoromethoxy)phenyl |
| A-182 | 2-(trifluoromethoxy)-3-fluorophenyl |
| A-183 | 2-(trifluoromethoxy)-4-fluorophenyl |
| A-184 | 2-(trifluoromethoxy)-5-fluorophenyl |
| A-185 | 2-(trifluoromethoxy)-6-fluorophenyl |
| A-186 | 3-(trifluoromethoxy)-4-fluorophenyl |
| A-187 | 3-(trifluoromethoxy)-5-fluorophenyl |
| A-188 | 2-fluoro-3-(trifluoromethoxy)phenyl |
| A-189 | 2-fluoro-4-(trifluoromethoxy)phenyl |
| A-190 | 2-fluoro-5-(trifluoromethoxy)phenyl |
| A-191 | 3-fluoro-4-(trifluoromethoxy)phenyl |
| A-192 | 2-(difluoromethoxy)-3-chlorophenyl |
| A-193 | 2-(difluoromethoxy)-4-chlorophenyl |
| A-194 | 2-(difluoromethoxy)-5-chlorophenyl |
| A-195 | 2-(difluoromethoxy)-6-chlorophenyl |
| A-196 | 3-(difluoromethoxy)-4-chlorophenyl |
| A-197 | 3-(difluoromethoxy)-5-chlorophenyl |
| A-198 | 2-chloro-3-(difluoromethoxy)phenyl |
| A-199 | 2-chloro-4-(difluoromethoxy)phenyl |
| A-200 | 2-chloro-5-(difluoromethoxy)phenyl |
| A-201 | 3-chloro-4-(difluoromethoxy)phenyl |
| A-202 | 2-(difluoromethoxy)-3-fluorophenyl |
| A-203 | 2-(difluoromethoxy)-4-fluorophenyl |
| A-204 | 2-(difluoromethoxy)-5-fluorophenyl |
| A-205 | 2-(difluoromethoxy)-6-fluorophenyl |
| A-206 | 3-(difluoromethoxy)-4-fluorophenyl |
| A-207 | 3-(difluoromethoxy)-5-fluorophenyl |
| A-208 | 2-fluoro-3-(difluoromethoxy)phenyl |
| A-209 | 2-fluoro-4-(difluoromethoxy)phenyl |
| A-210 | 2-fluoro-5-(difluoromethoxy)phenyl |
| A-211 | 3-fluoro-4-(difluoromethoxy)phenyl |
| A-212 | 2-(trifluoromethylthio)-3-chlorophenyl |
| A-213 | 2-(trifluoromethylthio)-4-chlorophenyl |
| A-214 | 2-(trifluoromethylthio)-5-chlorophenyl |
| A-215 | 2-(trifluoromethylthio)-6-chlorophenyl |
| A-216 | 3-(trifluoromethylthio)-4-chlorophenyl |
| A-217 | 3-(trifluoromethylthio)-5-chlorophenyl |
| A-218 | 2-chloro-3-(trifluoromethylthio)phenyl |
| A-219 | 2-chloro-4-(trifluoromethylthio)phenyl |
| A-220 | 2-chloro-5-(trifluoromethylthio)phenyl |
| A-221 | 3-chloro-4-(trifluoromethylthio)phenyl |
| A-222 | 2-(trifluoromethylthio)-3-fluorophenyl |
| A-223 | 2-(trifluoromethylthio)-4-fluorophenyl |
| A-224 | 2-(trifluoromethylthio)-5-fluorophenyl |
| A-225 | 2-(trifluoromethylthio)-6-fluorophenyl |
| A-226 | 3-(trifluoromethylthio)-4-fluorophenyl |
| A-227 | 3-(trifluoromethylthio)-5-fluorophenyl |
| A-228 | 2-fluoro-3-(trifluoromethylthio)phenyl |
| A-229 | 2-fluoro-4-(trifluoromethylthio)phenyl |
| A-230 | 2-fluoro-5-(trifluoromethylthio)phenyl |
| A-231 | 3-fluoro-4-(trifluoromethylthio)phenyl |
| A-232 | 2,3,4-trichlorophenyl |
| A-233 | 2,3,5-trichlorophenyl |
| A-234 | 2,3,6-trichlorophenyl |
| A-235 | 2,4,5-trichlorophenyl |
| A-236 | 2,4,6-trichlorophenyl |
| A-237 | 3,4,5-trichlorophenyl |
| A-238 | 2,3,4-trifluorophenyl |
| A-239 | 2,3,5-trifluorophenyl |
| A-240 | 2,3,6-trifluorophenyl |
| A-241 | 2,4,5-trifluorophenyl |
| A-242 | 2,4,6-trifluorophenyl |
| A-243 | 3,4,5-trifluorophenyl |
| A-244 | 2,3,4-trimethylphenyl |
| A-245 | 2,3,5-trimethylphenyl |
| A-246 | 2,3,6-trimethylphenyl |
| A-247 | 2,4,5-trimethylphenyl |
| A-248 | 2,4,6-trimethylphenyl |
| A-249 | 3,4,5-trimethylphenyl |
| A-250 | 2,3,4-trimethoxyphenyl |
| A-251 | 2,3,5-trimethoxyphenyl |
| A-252 | 2,3,6-trimethoxyphenyl |
| A-253 | 2,4,5-trimethoxyphenyl |
| A-254 | 2,4,6-trimethoxyphenyl |
| A-255 | 3,4,5-trimethoxyphenyl |

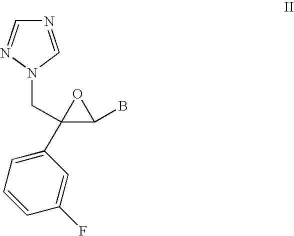

In particular with a view to their use, preference is given to the compounds II compiled in Table B below.

TABLE B

| Compound | Variable B is |
|---|---|
| II-1 | phenyl |
| II-2 | 2-ethylphenyl |
| II-3 | 3-ethylphenyl |
| II-4 | 4-ethylphenyl |
| II-5 | 2-trifluoromethylphenyl |
| II-6 | 3-trifluoromethylphenyl |
| II-7 | 4-trifluoromethylphenyl |
| II-8 | 2-trifluoromethoxyphenyl |
| II-9 | 3-trifluoromethoxyphenyl |
| II-10 | 4-trifluoromethoxyphenyl |
| II-11 | 2-difluoromethoxyphenyl |
| II-12 | 3-difluoromethoxyphenyl |
| II-13 | 4-difluoromethoxyphenyl |
| II-14 | 2-trifluoromethylthiophenyl |
| II-15 | 3-trifluoromethylthiophenyl |
| II-16 | 4-trifluoromethylthiophenyl |
| II-17 | 2,5-dichlorophenyl |
| II-18 | 2,6-dichlorophenyl |
| II-19 | 3,5-dichlorophenyl |
| II-20 | 2,5-difluorophenyl |
| II-21 | 2,6-difluorophenyl |
| II-22 | 3,4-difluorophenyl |
| II-23 | 3,5-difluorophenyl |
| II-24 | 2,3-dimethylphenyl |
| II-25 | 2,4-dimethylphenyl |
| II-26 | 2,5-dimethylphenyl |
| II-27 | 2,6-dimethylphenyl |
| II-28 | 3,4-dimethylphenyl |
| II-29 | 3,5-dimethylphenyl |
| II-30 | 2,3-diethylphenyl |
| II-31 | 2,4-diethylphenyl |
| II-32 | 2,5-diethylphenyl |
| II-33 | 2,6-diethylphenyl |
| II-34 | 3,5-diethylphenyl |
| II-35 | 3,4-diethylphenyl |
| II-36 | 2,3-dimethoxyphenyl |
| II-37 | 2,4-dimethoxyphenyl |
| II-38 | 2,5-dimethoxyphenyl |
| II-39 | 2,6-dimethoxyphenyl |
| II-40 | 3,4-dimethoxyphenyl |
| II-41 | 3,5-dimethoxyphenyl |
| II-42 | 2,3-di(trifluoromethyl)phenyl |
| II-43 | 2,4-di(trifluoromethyl)phenyl |
| II-44 | 2,5-di(trifluoromethyl)phenyl |
| II-45 | 2,6-di(trifluoromethyl)phenyl |
| II-46 | 3,4-di(trifluoromethyl)phenyl |
| II-47 | 3,5-di(trifluoromethyl)phenyl |
| II-48 | 2,3-di(trifluoromethoxy)phenyl |
| II-49 | 2,4-di(trifluoromethoxy)phenyl |
| II-50 | 2,5-di(trifluoromethoxy)phenyl |
| II-51 | 2,6-di(trifluoromethoxy)phenyl |
| II-52 | 3,4-di(trifluoromethoxy)phenyl |
| II-53 | 3,5-di(trifluoromethoxy)phenyl |
| II-54 | 2,3-di(difluoromethoxy)phenyl |
| II-55 | 2,4-di(difluoromethoxy)phenyl |
| II-56 | 2,5-di(difluoromethoxy)phenyl |
| II-57 | 2,6-di(difluoromethoxy)phenyl |
| II-58 | 3,4-di(difluoromethoxy)phenyl |
| II-59 | 3,5-di(difluoromethoxy)phenyl |
| II-60 | 2,3-di(trifluoromethylthio)phenyl |
| II-61 | 2,4-di(trifluoromethylthio)phenyl |
| II-62 | 2,5-di(trifluoromethylthio)phenyl |
| II-63 | 2,6-di(trifluoromethylthio)phenyl |
| II-64 | 3,4-di(trifluoromethylthio)phenyl |
| II-65 | 3,5-di(trifluoromethylthio)phenyl |
| II-66 | 2-fluoro-3-chlorophenyl |
| II-67 | 2-fluoro-4-chlorophenyl |
| II-68 | 2-fluoro-5-chlorophenyl |
| II-69 | 2-fluoro-6-chlorophenyl |
| II-70 | 3-fluoro-4-chlorophenyl |
| II-71 | 3-fluoro-5-chlorophenyl |
| II-72 | 2-chloro-5-fluorophenyl |
| II-73 | 3-chloro-4-fluorophenyl |
| II-74 | 2-methyl-3-chlorophenyl |
| II-75 | 2-methyl-4-chlorophenyl |
| II-76 | 2-methyl-5-chlorophenyl |
| II-77 | 2-methyl-6-chlorophenyl |
| II-78 | 3-methyl-4-chlorophenyl |
| II-79 | 3-methyl-5-chlorophenyl |
| II-80 | 2-chloro-3-methylphenyl |
| II-81 | 2-chloro-4-methylphenyl |
| II-82 | 2-chloro-5-methylphenyl |
| II-83 | 3-chloro-4-methylphenyl |
| II-84 | 2-methyl-3-fluorophenyl |
| II-85 | 2-methyl-4-fluorophenyl |
| II-86 | 2-methyl-5-fluorophenyl |
| II-87 | 2-methyl-6-fluorophenyl |
| II-88 | 3-methyl-4-fluorophenyl |
| II-89 | 3-methyl-5-fluorophenyl |
| II-90 | 2-fluoro-3-methylphenyl |
| II-91 | 2-fluoro-4-methylphenyl |
| II-92 | 2-fluoro-5-methylphenyl |
| II-93 | 3-fluoro-4-methylphenyl |
| II-94 | 2-chloro-3-ethylphenyl |
| II-95 | 2-chloro-4-ethylphenyl |
| II-96 | 2-chloro-5-ethylphenyl |
| II-97 | 3-chloro-4-ethylphenyl |
| II-98 | 2-ethyl-3-chlorophenyl |
| II-99 | 2-ethyl-4-chlorophenyl |
| II-100 | 2-ethyl-5-chlorophenyl |
| II-101 | 2-ethyl-6-chlorophenyl |
| II-102 | 2-ethyl-3-fluorophenyl |
| II-103 | 2-ethyl-4-fluorophenyl |
| II-104 | 2-ethyl-5-fluorophenyl |
| II-105 | 2-ethyl-6-fluorophenyl |
| II-106 | 3-ethyl-4-fluorophenyl |
| II-107 | 3-ethyl-5-fluorophenyl |
| II-108 | 2-fluoro-3-ethylphenyl |
| II-109 | 2-fluoro-4-ethylphenyl |
| II-110 | 2-fluoro-5-ethylphenyl |
| II-111 | 3-fluoro-4-ethylphenyl |
| II-112 | 2-methoxy-3-chlorophenyl |
| II-113 | 2-methoxy-4-chlorophenyl |
| II-114 | 2-methoxy-5-chlorophenyl |
| II-115 | 2-methoxy-6-chlorophenyl |
| II-116 | 3-methoxy-4-chlorophenyl |
| II-117 | 3-methoxy-5-chlorophenyl |
| II-118 | 2-chloro-5-methoxyphenyl |
| II-119 | 3-chloro-4-methoxyphenyl |
| II-120 | 2-methoxy-3-fluorophenyl |
| II-121 | 2-methoxy-4-fluorophenyl |
| II-122 | 2-methoxy-5-fluorophenyl |
| II-123 | 2-methoxy-6-fluorophenyl |
| II-124 | 3-methoxy-4-fluorophenyl |
| II-125 | 3-methoxy-5-fluorophenyl |
| II-126 | 2-fluoro-3-methoxyphenyl |
| II-127 | 2-fluoro-4-methoxyphenyl |
| II-128 | 2-fluoro-5-methoxyphenyl |
| II-129 | 3-fluoro-4-methoxyphenyl |
| II-130 | 3-fluoro-5-methoxyphenyl |
| II-131 | 2-(trifluoromethyl)-3-chlorophenyl |
| II-132 | 2-(trifluoromethyl)-4-chlorophenyl |
| II-133 | 2-(trifluoromethyl)-5-chlorophenyl |
| II-134 | 2-(trifluoromethyl)-6-chlorophenyl |
| II-135 | 3-(trifluoromethyl)-4-chlorophenyl |
| II-136 | 3-(trifluoromethyl)-5-chlorophenyl |
| II-137 | 2-chloro-3-(trifluoromethyl)phenyl |
| II-138 | 2-chloro-4-(trifluoromethyl)phenyl |
| II-139 | 2-chloro-5-(trifluoromethyl)phenyl |
| II-140 | 3-chloro-4-(trifluoromethyl)phenyl |
| II-141 | 2-(trifluoromethyl)-3-fluorophenyl |
| II-142 | 2-(trifluoromethyl)-4-fluorophenyl |
| II-143 | 2-(trifluoromethyl)-5-fluorophenyl |
| II-144 | 2-(trifluoromethyl)-6-fluorophenyl |
| II-145 | 3-(trifluoromethyl)-4-fluorophenyl |
| II-146 | 3-(trifluoromethyl)-5-fluorophenyl |
| II-147 | 2-fluoro-3-(trifluoromethyl)phenyl |
| II-148 | 2-fluoro-4-(trifluoromethyl)phenyl |
| II-149 | 2-fluoro-5-(trifluoromethyl)phenyl |
| II-150 | 3-fluoro-4-(trifluoromethyl)phenyl |
| II-151 | 2-(trifluoromethoxy)-3-chlorophenyl |
| II-152 | 2-(trifluoromethoxy)-4-chlorophenyl |
| II-153 | 2-(trifluoromethoxy)-5-chlorophenyl |
| II-154 | 2-(trifluoromethoxy)-6-chlorophenyl |
| II-155 | 3-(trifluoromethoxy)-4-chlorophenyl |
| II-156 | 3-(trifluoromethoxy)-5-chlorophenyl |
| II-157 | 2-chloro-3-(trifluoromethoxy)phenyl |
| II-158 | 2-chloro-4-(trifluoromethoxy)phenyl |
| II-159 | 2-chloro-5-(trifluoromethoxy)phenyl |
| II-160 | 3-chloro-4-(trifluoromethoxy)phenyl |
| II-161 | 2-(trifluoromethoxy)-3-fluorophenyl |
| II-162 | 2-(trifluoromethoxy)-4-fluorophenyl |
| II-163 | 2-(trifluoromethoxy)-5-fluorophenyl |
| II-164 | 2-(trifluoromethoxy)-6-fluorophenyl |
| II-165 | 3-(trifluoromethoxy)-4-fluorophenyl |
| II-166 | 3-(trifluoromethoxy)-5-fluorophenyl |
| II-167 | 2-fluoro-3-(trifluoromethoxy)phenyl |
| II-168 | 2-fluoro-4-(trifluoromethoxy)phenyl |
| II-169 | 2-fluoro-5-(trifluoromethoxy)phenyl |
| II-170 | 3-fluoro-4-(trifluoromethoxy)phenyl |
| II-171 | 2-(difluoromethoxy)-3-chlorophenyl |
| II-172 | 2-(difluoromethoxy)-4-chlorophenyl |
| II-173 | 2-(difluoromethoxy)-5-chlorophenyl |
| II-174 | 2-(difluoromethoxy)-6-chlorophenyl |
| II-175 | 3-(difluoromethoxy)-4-chlorophenyl |
| II-176 | 3-(difluoromethoxy)-5-chlorophenyl |
| II-177 | 2-chloro-3-(difluoromethoxy)phenyl |
| II-178 | 2-chloro-4-(difluoromethoxy)phenyl |
| II-179 | 2-chloro-5-(difluoromethoxy)phenyl |
| II-180 | 3-chloro-4-(difluoromethoxy)phenyl |
| II-181 | 2-(difluoromethoxy)-3-fluorophenyl |
| II-182 | 2-(difluoromethoxy)-4-fluorophenyl |
| II-183 | 2-(difluoromethoxy)-5-fluorophenyl |
| II-184 | 2-(difluoromethoxy)-6-fluorophenyl |
| II-185 | 3-(difluoromethoxy)-4-fluorophenyl |
| II-186 | 3-(difluoromethoxy)-5-fluorophenyl |
| II-187 | 2-fluoro-3-(difluoromethoxy)phenyl |
| II-188 | 2-fluoro-4-(difluoromethoxy)phenyl |
| II-189 | 2-fluoro-5-(difluoromethoxy)phenyl |
| II-190 | 3-fluoro-4-(difluoromethoxy)phenyl |
| II-191 | 2-(trifluoromethylthio)-3-chlorophenyl |
| II-192 | 2-(trifluoromethylthio)-4-chlorophenyl |
| II-193 | 2-(trifluoromethylthio)-5-chlorophenyl |
| II-194 | 2-(trifluoromethylthio)-6-chlorophenyl |
| II-195 | 3-(trifluoromethylthio)-4-chlorophenyl |
| II-196 | 3-(trifluoromethylthio)-5-chlorophenyl |
| II-197 | 2-chloro-3-(trifluoromethylthio)phenyl |
| II-198 | 2-chloro-4-(trifluoromethylthio)phenyl |
| II-199 | 2-chloro-5-(trifluoromethylthio)phenyl |
| II-200 | 3-chloro-4-(trifluoromethylthio)phenyl |
| II-201 | 2-(trifluoromethylthio)-3-fluorophenyl |
| II-202 | 2-(trifluoromethylthio)-4-fluorophenyl |
| II-203 | 2-(trifluoromethylthio)-5-fluorophenyl |
| II-204 | 2-(trifluoromethylthio)-6-fluorophenyl |
| II-205 | 3-(trifluoromethylthio)-4-fluorophenyl |
| II-206 | 3-(trifluoromethylthio)-5-fluorophenyl |
| II-207 | 2-fluoro-3-(trifluoromethylthio)phenyl |
| II-208 | 2-fluoro-4-(trifluoromethylthio)phenyl |
| II-209 | 2-fluoro-5-(trifluoromethylthio)phenyl |
| II-210 | 3-fluoro-4-(trifluoromethylthio)phenyl |
| II-211 | 2,3,4-trichlorophenyl |
| II-212 | 2,3,5-trichlorophenyl |
| II-213 | 2,3,6-trichlorophenyl |
| II-214 | 2,4,5-trichlorophenyl |
| II-215 | 2,4,6-trichlorophenyl |
| II-216 | 3,4,5-trichlorophenyl |
| II-217 | 2,3,4-trifluorophenyl |
| II-218 | 2,3,5-trifluorophenyl |
| II-219 | 2,3,6-trifluorophenyl |
| II-220 | 2,4,5-trifluorophenyl |
| II-221 | 2,4,6-trifluorophenyl |
| II-222 | 3,4,5-trifluorophenyl |
| II-223 | 2,3,4-trimethylphenyl |
| II-224 | 2,3,5-trimethylphenyl |
| II-225 | 2,3,6-trimethylphenyl |
| II-226 | 2,4,5-trimethylphenyl |
| II-227 | 2,4,6-trimethylphenyl |
| II-228 | 3,4,5-trimethylphenyl |
| II-229 | 2,3,4-trimethoxyphenyl |
| II-230 | 2,3,5-trimethoxyphenyl |
| II-231 | 2,3,6-trimethoxyphenyl |
| II-232 | 2,4,5-trimethoxyphenyl |
| II-233 | 2,4,6-trimethoxyphenyl |
| II-234 | 3,4,5-trimethoxyphenyl |

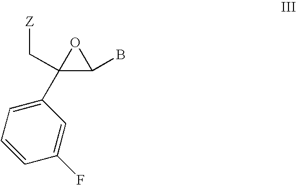

Particular preference is given to the compounds III compiled in Tables 1b to 5b below. The groups mentioned for a substituent in the tables are furthermore per se, independently of the combination in which they are mentioned, a particularly preferred aspect of the substituent in question.

### Table 1b

Compounds III in which Z is Cl and B corresponds in each case to one row of Table A (compounds III.1bA-1 to III.1bA-255)

### Table 2b

Compounds III in which Z is OTs and B corresponds in each case to one row of Table A (compounds III.2bA-1 to III.2bA-255)

### Table 3b

Compounds III in which Z is OMs and B corresponds in each case to one row of Table A (compounds III.3bA-1 to III.3bA-255)

### Table 4b

Compounds III in which Z is OH and B corresponds in each case to one row of Table A (compounds III.4bA-1 to III.4bA-255)

### Table 5b

Compounds III in which Z is Br and B corresponds in each case to one row of Table A (compounds III.5bA-1 to III.5bA-255)

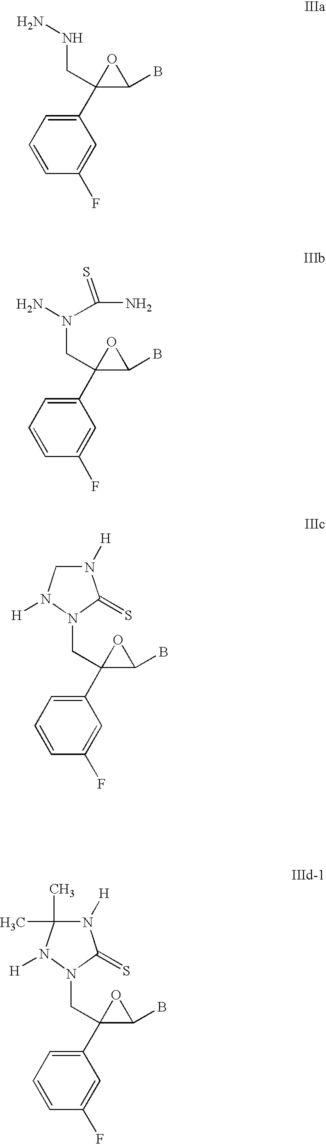

Particular preference is given to the compounds IIIa, IIIb, IIIc and IIId-1 according to the invention compiled in Tables 1c to 4c below. The groups mentioned for a substituent in the tables are furthermore per se, independently of the combination in which they are mentioned, a particularly preferred group of the substituent in question.

### Table 1c

Compounds IIIa in which B corresponds in each case to one row of Table A (compounds IIIa.1cA-1 to IIIa.1cA-255)

### Table 2c

Compounds IIIb in which B corresponds in each case to one row of Table A (compounds IIIb.2cA-1 to IIIb.2cA-255)

### Table 3c

Compounds IIIc in which B corresponds in each case to one row of Table A (compounds IIIc.3cA-1 to IIIc.3cA-255)

### Table 4c

Compounds IIId-1 in which B corresponds in each case to one row of Table A (compounds IIId-1.4cA-1 to IIId-1.4cA-255)

From the tables above, the compound names for the individual compounds can be derived as follows: the “compound I.3aA-10” (emphases added), for example, is the compound of the formula I according to the invention in which D is S—C₂H₅ (as stated in Table 3a) and B is 4-methylphenyl (as stated in row 10 of Table A).

The compounds of the invention (compounds according to the invention), in particular the compounds of the formulae I and II, and their compositions according to the invention are suitable as fungicides for controlling harmful fungi. They are distinguished by an excellent activity against a broad spectrum of phytopathogenic fungi including soil-borne pathogens, which originate, in particular, from the classes of the Plasmodiophoromycetes, Peronosporomycetes (syn. Oomycetes), Chytridiomycetes, Zygomycetes, Ascomycetes, Basidiomycetes and Deuteromycetes (syn. Fungi imperfecti). Some of them are systemically effective and can be used in crop protection as foliar fungicides, as fungicides for seed dressing and as soil fungicides. Furthermore, they are suitable for controlling fungi which, inter alia, attack the wood or the roots of plants.

The compounds according to the invention are particularly important in the control of a multitude of pathogenic fungi on various crop plants, such as cereal, e.g. wheat, rye, barley, triticale, oats or rice, beet, e.g. sugar or feed beet; pomaceous fruit, stone and soft fruit, e.g. apples, pears, plums, peaches, almonds, cherries, strawberries, raspberries, currants or gooseberries; leguminous plants, e.g. beans, lentils, peas, alfalfa or soya; oil plants, e.g. rapeseed, mustard, olives, sunflowers, coconut, cocoa, castor beans, oil palm, peanuts or soya; cucurbits, e.g. pumpkins, cucumbers or melons; fiber plants, e.g. cotton, flax, hemp or jute; citrus fruit, e.g. oranges, lemons, grapefruit or mandarins; vegetable plants, e.g. spinach, lettuce, asparagus, cabbage plants, carrots, onions, tomatoes, potatoes, pumpkins or paprika; laurel plants, e.g. avocados, cinnamon or camphor; energy plants and raw material plants, e.g. corn, soya, wheat, rapeseed, sugarcane or oil palm; corn; tobacco; nuts; coffee; tea; bananas; grapes (table grapes and wine grapes); hops; grass, e.g. turf; rubber plants; ornamental and forest plants, e.g. flowers, shrubs, deciduous trees and conifer trees and on the propagation material, e.g. seeds, and the crops of these plants.

Preferably, the compounds I according to the invention or the compositions according to the invention are used for the control of a multiplicity of fungal pathogens in agricultural cultivation, e.g. potatoes, sugar beet, tobacco, wheat, rye, barley, oats, rice, corn, cotton, soya, rapeseed, pulses, sunflowers, coffee or sugarcane; fruit plants, grape plants and ornamental plants and vegetable plants, e.g. cucumbers, tomatoes, beans and pumpkins, and on the propagation material, e.g. seeds, and the crops of these plants.

The term plant propagation materials comprises all generative parts of the plant, e.g. seeds, and vegetative plant parts, such as seedlings and tubers (e.g. potatoes) that can be used for the propagation of a plant. These include seeds, roots, fruits, tubers, bulbs, rhizomes, shoots and other plant parts, including seedlings and young plants, which are transplanted after germination or emergence. The young plants can be protected from harmful fungi by a partial or complete treatment, e.g. by immersing or watering.

The treatment of plant propagation materials with the compounds according to the invention or the compositions according to the invention is employed for the control of a multiplicity of fungal pathogens in cereal cultures, e.g. wheat, rye, barley or oats; rice, corn, cotton and soya.

The term crop plants also includes that those plants which have been modified by breeding, mutagenesis or genetic engineering methods including the biotechnological agricultural products that are on the market or in development (see, for example, http://www.bio.org/speeches/pubs/er/agri_products.asp). Genetically modified plants are plants whose genetic material has been modified in a manner such as does not occur under natural conditions by crossing, mutations or natural recombination (i.e. reconfiguration of the genetic information). Here, generally one or more genes are integrated into the hereditary material of the plant in order to improve the properties of the plant. Such genetic modifications also comprise post-translational modifications of proteins, oligo- or polypeptides e.g. by means of glycosylation or binding of polymers such as, for example, prenylated, acetylated or farnesylated radicals or PEG radicals.

By way of example, plants may be mentioned that have acquired by breeding and genetic engineering measures a tolerance to certain classes of herbicide, such as hydroxyphenylpyruvate dioxygenase (HPPD) inhibitors, acetolactate synthase (ALS) inhibitors such as, for example, sulfonylureas (EP-A 257 993, U.S. Pat. No. 5,013,659) or imidazolinones (e.g. U.S. Pat. No. 6,222,100, WO 01/82685, WO 00/26390, WO 97/41218, WO 98/02526, WO 98/02527, WO 04/106529, WO 05/20673, WO 03/14357, WO 03/13225, WO 03/14356, WO 04/16073), enolpyruvylshikimate-3-phosphate synthase (EPSPS) inhibitors such as, for example, glyphosate (see, for example, WO 92/00377), glutamine synthetase (GS) inhibitors such as e.g. glufosinate (see, for example, EP-A 242 236, EP-A 242 246) or oxynil herbicides (see, for example, U.S. Pat. No. 5,559,024). By means of breeding and mutagenesis, Clearfield® rapeseed (BASF SE, Germany), for example, has been produced which has a tolerance to imidazolinones, e.g. imazamox. With the aid of genetic engineering methods, crop plants, such as soya, cotton, corn, beet and rapeseed, have been produced that are resistant to glyphosate or glufosinate, which are obtainable under the trade names RoundupReady® (glyphosate-resistant, Monsanto, U.S.A.) and Liberty Link® (glufosinate-resistant, Bayer CropScience, Germany).

Furthermore, plants are also comprised that, with the aid of genetic engineering measures, produce one or more toxins, e.g. those from the bacterial strain Bacillus. Toxins that are produced by such genetically modified plants comprise, for example, insecticidal proteins of Bacillus spp., in particular of B. thuringiensis, such as the endotoxins Cry1Ab, Cry1Ac, Cry1F, Cry1Fa2, Cry2Ab, Cry3A, Cry3Bb1, Cry9c, Cry34Ab1 or Cry35Ab1; or vegetative insecticidal proteins (VIPs), e.g. VIP1, VIP2, VIP3, or VIP3A; insecticidal proteins of nematode-colonizing bacteria, e.g. Photorhabdus spp. or Xenorhabdus spp.; toxins from animal organisms, e.g. wasp, spider or scorpion toxins; fungal toxins, e.g. from streptomycetes; plant lectins, e.g. from pea or barley; agglutinins; proteinase inhibitors, e.g. trypsin inhibitors, serine protease inhibitors, patatin, cystatin or papain inhibitors; ribosome-inactivating proteins (RIPs), e.g. ricin, corn RIP, abrin, luffin, saporin or bryodin; steroid-metabolizing enzymes, e.g. 3-hydroxysteroid oxidase, ecdysteroid IDP glycosyl transferase, cholesterol oxidase, ecdysone inhibitors or HMG-CoA reductase; ion channel blockers, e.g. inhibitors of sodium channels or calcium channels; juvenile hormone esterase; receptors for the diuretic hormone (helicokinin receptors); stilbene synthase, bibenzyl synthase, chitinases and glucanases. These toxins can be produced in the plants also as pretoxins, hybrid proteins, truncated or otherwise modified proteins. Hybrid proteins are distinguished by a new combination of various protein domains (see e.g. WO 2002/015701). Further examples of toxins of this type or genetically modified plants that produce these toxins are disclosed in EP-A 374 753, WO 93/07278, WO 95/34656, EP-A 427 529, EP-A 451 878, WO 03/18810 and WO 03/52073. The methods for the production of these genetically modified plants are known to the person skilled in the art and presented, for example, in the abovementioned publications. Numerous examples of the aforementioned toxins impart, to the plants which produce these, a tolerance to pests from all taxonomic classes of arthropods, in particular to beetles (Coeleropta), Diptera and butterflies (Lepidoptera) and to nematodes (Nematoda). Genetically modified plants that produce one or more genes that code for insecticidal toxins are described, far example, in the abovementioned publications and are in some cases commercially obtainable, such as, for example, YieldGard® (types of corn that produce the toxin Cry1Ab), YieldGard® Plus (types of corn that produce the toxins Cry1Ab and Cry3Bb1), Starlink® (types of corn that produce the toxin Cry9c), Herculex® RW (types of corn that produce the toxins Cry34Ab1, Cry35Ab1 and the enzyme phosphinothricin-N-acetyltransferase [PAT]); NuCOTN® 33B (types of cotton that produce the toxin Cry1Ac), Bollgard® I (types of cotton that produce the toxin Cry1Ac), Bollgard® II (types of cotton that produce the toxins Cry1Ac and Cry2Ab2); VIPCOT® (types of cotton that produce a VIP toxin); NewLeaf® (types of potato that produce the toxin Cry3A); Bt-Xtra®, NatureGard®, KnockOut®, BiteGard®, Protecta®, Bt11 (e.g. Agrisure® CB) and Bt176 from Syngenta Seeds SAS, France, (types of corn that produce the toxin Cry1Ab and the PAT enzyme), MIR604 from Syngenta Seeds SAS, France (types of corn that produce a modified version of the toxin Cry3A, in this respect see WO 03/018810), MON 863 from Monsanto Europe S.A., Belgium (types of corn that produce the toxin Cry3Bb1), IPC 531 from Monsanto Europe S.A., Belgium (types of cotton that produce a modified version of the toxin Cry1Ac) and 1507 from Pioneer Overseas Corporation, Belgium (types of corn that produce the toxin Cry1F and the PAT enzyme).

Furthermore, plants are also comprised that with the aid of genetic engineering measures produce one or more proteins that have an increased resistance or hardiness to bacterial, viral or fungal pathogens, such as, for example, “Pathogenesis-related proteins” (PR proteins, see EP-A 0 392 225), resistance proteins (e.g. types of potato that produce two resistance genes to Phytophthora infestans from the Mexican wild potato Solanum bulbocastanum) or T4 lysozyme (e.g. types of potato that as a result of the production of this protein are resistant to bacteria such as Erwinia amylvora).

Furthermore, types of plants are also comprised whose productivity has been improved with the aid of genetic engineering methods by increasing, for example, the productivity (e.g. biomass, grain yield, starch, oil or protein content), the tolerance to drought, salt or other restrictive environmental factors or the hardiness to pests and fungal, bacterial and viral pathogens.

Furthermore, plants are also comprised whose materials have been modified, in particular for improving human or animal nutrition, with the aid of genetic engineering methods by, for example, oil plants producing health-promoting long-chain omega-3 fatty acids or monounsaturated omega-9 fatty acids (e.g. Nexera® rape seed, DOW Agro Sciences, Canada).

Furthermore, plants are also comprised which have been modified for the improved production of raw materials with the aid of genetic engineering methods by, for example, increasing the amylopectin content of potatoes (Amflora® potato, BASF SE, Germany).

The present invention therefore also comprises the use of compounds according to the invention or their compositions for the treatment of transgenic plants, in particular transgenic soya plants or transgenic corn plants. Transgenic plants are plants such as described above that have been modified with the aid of genetic engineering methods, in particular plants whose properties have been improved with the aid of genetic engineering methods. The invention in particular comprises the use of compounds according to the invention or their compositions for the treatment of transgenic plants that are resistant to glyphosate, glufosinate or glufosinate-ammonium. In a further embodiment, the invention also comprises the use of compounds according to the invention or their compositions for the treatment of herbicide-resistant plants. In a further embodiment, the invention also comprises the use of compounds according to the invention or their compositions for the treatment of herbicide-sensitive plants.

Specifically, the compounds according to the invention or their compositions according to the invention are suitable for the control of the following plant diseases:
Albugo spp. (white rust) on ornamental plants, vegetable crops (e.g. A. candida) and sunflowers (e.g. A. tragopogonis); Alternaria spp. (blackness, black spot) on vegetables, rapeseed (e.g. A. brassicola or A. brassicae), sugar beet (e.g. A. tenuis), fruit, rice, soybeans, and on potatoes (e.g. A. solani or A. alternate) and tomatoes (e.g. A. solani or A. alternate) and Alternaria spp. (cereal black) on wheat; Aphanomyces spp. on sugar beet and vegetables; Ascochyta spp. on cereal and vegetables, e.g. A. tritici (leaf blotch) on wheat and A. hordei on barley; Bipolaris and Drechslera spp. (Teleomorph: Cochliobolus spp.) e.g. leaf spot diseases (D. maydis and B. zeicola) on corn, e.g. brown spot (B. sorokiniana) on cereal and, for example, B. ayzae on rice and on turf; Blumeria (formerly: Erysiphe) graminis (powdery mildew) on cereal (e.g. wheat or barley); Botryosphaeria spp. (‘Black Dead Arm disease’) on grapevines (e.g. B. obtuse); Botrytis cinerea (Teleomorph: Botryotinia fuckeliana: gray mold, gray rot) on soft fruit and pomaceous fruit (inter alia strawberries), vegetables (inter alia lettuce, carrots, celery and cabbage), rapeseed, flowers, grapevines, forest crops and wheat (cereal mold); Bremia lactucae (downy mildew) on lettuce; Ceratocystis (Syn. Ophiostoma) spp. (blue fungus) on deciduous trees and coniferous trees, e.g. C. ulmi (elm death, Dutch elm disease) on elms; Cercospora spp. (Cercospora leaf spots) on corn (e.g. C. zeae-maydis), rice, sugar beet (e.g. C. beticola), sugar cane, vegetables, coffee, soybeans (e.g. C. sojina or C. kikuchii) and rice; Cladosporium spp. on tomato (e.g. C. fulvum: velvet spot disease) and cereal, e.g. C. herbarum (cereal black) on wheat; Claviceps purpurea (ergot) on cereal; Cochliobolus (Anamorph: Helminthosporium or Bipolaris) spp. (leaf spots) on corn (e.g. C. carbonum), cereal (e.g. C. sativus, Anamorph: B. sorokiniana: brown spot) and rice (e.g. C. miyabeanus, Anamorph: H. oryzae); Colletotrichum (Teleomorph: Glomerella) spp. (scorch spots, anthracnose) on cotton (e.g. C. gossypii), corn (e.g. C. graminicola: stem rot and scorch spots), soft fruit, potatoes (e.g. C. coccodes: wilt), bean (e.g. C. lindemuthianum) and soybeans (e.g. C. truncatum); Corticium spp., e.g. C. sasakii (sheath blight) on rice; Corynespora cassiicola (leaf spots) on soybeans and ornamental plants; Cycloconium spp., e.g. C. oleaginum on olive; Cylindrocarpon spp. (e.g. fruit tree cancer or vine death, Teleomorph: Nectria or Neonectria spp.) on fruit trees, grapevines (e.g. C. liriodendri, Teleomorph: Neonectria liriodendri, ‘Black Foot disease’) and many ornamental trees; Dematophora (Teleomorph: Rosellinia) necatrix (root/stem rot) on soybeans; Diaporthe spp. e.g. D. phaseolorum (stem disease) on soybeans; Drechslera (Syn. Helminthosporium, Teleomorph: Pyrenophora) spp. on corn, cereal, such as barley (e.g. D. teres, leaf spots) and on wheat (e.g. D. triticirepentis: DTR leaf blotch), rice and turf; Esca disease (vine stock death, apoplexy) on grapevines, caused by Formitiporia (Syn. phellinus) punctata, F. mediterranea, Phaeomoniella chlamydospora (formerly Phaeoacremonium chlamydosporum), Phaeoacremonium aleophilum and/or Botryosphaeria obtusa, Elsinoe spp. on pomaceous fruit (E. pyri) and soft fruit (E. veneta: scorch spots) and grapevines (E. ampelina: scorch spots); Entyloma oryzae (leaf scorch) on rice; Epicoccum spp. (cereal black) on wheat; Erysiphe spp. (powdery mildew) on sugar beet (E. betae), vegetables (e.g. E. pisi), such as cucurbits (e.g. E. cichoracearum) and cabbage plants, such as rapeseed (e.g. E. cruciferarum); Eutypa lata (Eutypa cancer or Eutypa death, Anamorph: Cytosporina lata, Syn. Libertella blepharis) on fruit trees, grapevines and many ornamental trees; Exserohilum (Syn. Helminthosporium) spp. on corn (e.g. E. turcicum); Fusarium (Teleomorph: Gibberella) spp. (wilt, root rot and stem rot) on various plants, such as, for example, F. graminearum or F. culmorum (root rot and empty or white head) on cereal (e.g. wheat or barley), F. oxysporum on tomatoes, F. solani on soybeans and F. verticillioides on corn; Gaeumannomyces graminis (black sheath rot) on cereal (e.g. wheat or barley) and corn; Gibberella spp. on cereal (e.g. G. zeae) and rice (e.g. G. fujikuroi: Bakanae disease); Glomerella cingulata on grapevines, pomaceous fruit and other plants and G. gossypii on cotton; Grainstaining complex on rice; Guignardia bidwellii (black rot) on grapevines; Gymnosporangium spp. on rosaceae and juniper, e.g. G. sabinae (pear trellis rust) on pears; Helminthosporium spp. (Syn. Drechslera, Teleomorph: Cochliobolus) on corn, cereal and rice; Hemileia spp., e.g. H. vastatrix (coffee leaf rust) on coffee; Isariopsis clavispora (Syn. Cladosporium vitis) on grapevines; Macrophomina phaseolina (Syn. phaseoli) (root/stem rot) on soybeans and cotton; Microdochium (Syn. Fusarium) nivale (snow mold) on cereal (e.g. wheat or barley); Microsphaera diffuse (powdery mildew) on soybeans; Monilinia spp., e.g. M. laxa, M fructicola and M. fructigena (blossom and tip blotch) on pomaceous fruit and other rosaceae; Mycosphaerella spp. on cereal, bananas, soft fruit and peanuts, such as, for example, M. graminicola (Anamorph: Septoria tritici, Septoria leaf blotch) on wheat or M. fijiensis (black Sigatoka disease) on bananas; Peronospora spp. (downy mildew) on cabbage (e.g. P. brassicae), rapeseed (e.g. P. parasitica), onion plants (e.g. P. destructor), tobacco (P. tabacina) and soybeans (e.g. P. manshurica); Phakopsora pachyrhizi and P. meibomiae (soybean rust) on soybeans; Phialophora spp. e.g. on grapevines (e.g. P. tracheiphila and P. tetraspora) and soybeans (e.g. P. gregata: stem disease); Phoma lingam (root rot and stem rot) on rapeseed and cabbage and P. betae (leaf spots) on sugar beet; Phomopsis spp. on sunflowers, grapevines (e.g. P. viticola: black spot disease) and soybeans (e.g. stem rot: P. phaseoli, Teleomorph: Diaporthe phaseolorum); Physoderma maydis (brown spot) on corn; Phytophthora spp. (wilt, root rot, leaf rot, stem rot and fruit rot) on various plants, such as on bellpepper and cucurbits (e.g. P. capsici), soybeans (e.g. P. megasperma, Syn: P. sojae), potatoes and tomatoes (e.g. P. infestans: haulm and brown rot) and deciduous trees (e.g. P. ramorum: sudden oak death); Plasmodiophora brassicae (cabbage hernia) on cabbage, rapeseed, radish and other plants; Plasmopara spp., e.g. P. viticola (vine Peronospora, downy mildew) on grapevines and P. halstedii on sunflowers; Podosphaera spp. (powdery mildew) on rosaceae, hops, pomaceous fruit and soft fruit, e.g. P. leucotricha on apple; Polymyxa spp., e.g. on cereal, such as barley and wheat (P. graminis) and sugar beet (P. betae) and the viral diseases transmitted thereby; Pseudocercosporella herpotrichoides (strawbreaker, Teleomorph: Tapesia yallundae) on cereal, e.g. wheat or barley; Pseudoperonospora (downy mildew) on various plants, e.g. P. cubensis on cucurbits or P. humili on hops; Pseudopezicula tracheiphila (red fire disease, Anamorph: Phialophora) on grapevines; Puccinia spp. (rust disease) on various plants, e.g. P. triticina (wheat brown rust), P. striiformis (yellow rust), P. hordei (dwarf rust), P. graminis (black rust) or P. recondite (rye brown rust) on cereal, such as, for example, wheat, barley or rye, and on asparagus (e.g. P. asparagi); Pyrenophora (Anamorph: Drechslera) tritici-repentis (leaf blotch) on wheat or P. teres (net blotch) on barley; Pyricularia spp., e.g. P. oryzae (Teleomorph: Magnaporthe grisea, rice-leaf scorch) on rice and P. grisea on turf and cereal; Pythium spp. (fall disease) on turf, rice, corn, wheat, cotton, rapeseed, sunflowers, sugar beet, vegetables and other plants (e.g. P. ultimum or P. aphanidermatum); Ramularia spp., e.g. R. collo-cygni (speckle disease/sunburn complex/physiological leaf spots) on barley and R. beticola on sugar beet; Rhizoctonia spp. on cotton, rice, potatoes, turf, corn, rapeseed, potatoes, sugar beet, vegetables and on various further plants, e.g. R. solani (root rot/stem rot) on soybeans, R. solani (leaf sheath blight) on rice or R. cerealis (sharp eye spot) on wheat or barley; Rhizopus stolonifer (soft rot) on strawberries, carrots, cabbage, grapevines and tomato; Rhynchosporium secalis (leaf spots) on barley, rye and triticale; Sarocladium oryzae and S. attenuatum (leaf sheath rot) on rice; Sclerotinia spp. (stem rot or white rot) on vegetables and agricultural crops, such as rapeseed, sunflowers (e.g. Sclerotinia sclerotiorum) and soybeans (e.g. S. rolfsii); Septoria spp. on various plants, e.g. S. glycines (leaf spots) on soybeans, S. tritici(Septoria leaf blotch) on wheat and S. (Syn. Stagonospora) nodorum (leaf and glume blotch) on cereal; Uncinula (Syn. Erysiphe) necator (powdery mildew, Anamorph: Oidium tuckeri) on grapevines; Setospaeria spp. (leaf spots) on corn (e.g. S. turcicum, Syn. Helminthosporium turcicum) and turf; Sphacelotheca spp. (smut) on corn, (e.g. S. reiliana: head smut), millet and sugar cane; Sphaerotheca fuliginea (powdery mildew) on cucurbits; Spongospora subterranea (powdery scab) on potatoes and the virus diseases transmitted thereby; Stagonospora spp. on cereal, e.g. S. nodorum (leaf and glume blotch, Teleomorph: Leptosphaeria [Syn. Phaeosphaeria] nodorum) on wheat; Synchytrium endobioticum on potatoes (potato cancer); Taphrina spp., e.g. T. deformans (curl disease) on peach and T. pruni (pockets disease) on plums; Thielaviopsis spp. (black root rot) on tobacco, pomaceous fruit, vegetable crops, soybeans and cotton, e.g. T. basicola (Syn. Chalara elegans); Tilletia spp. (smut or stinking smut) on cereal, such as, for example, T. tritici (Syn. T. caries, wheat smut) and T. controversa (dwarf smut) on wheat; Typhula incarnata (snow mold) on barley or wheat; Urocystis spp., e.g. U. occulta (stem scorch) on rye; Uromyces spp. (rust) on vegetable plants, such as beans (e.g. U. appendiculatus, Syn. U. phaseoli) and sugar beet (e.g. U. betae); Ustilago spp. (loose smut) on cereal (e.g. U. nuda and U. avaenae), corn (e.g. U. maydis: corn smut) and sugar cane; Venturia spp. (scab) on apples (e.g. V. inaequalis) and pears; and Verticillium spp. (foliage and shoot wilt) on various plants, such as fruit trees and ornamental trees, grapevines, soft fruit, vegetable crops and agricultural crops, such as, for example, V. dahliae on strawberries, rapeseed, potatoes and tomatoes.

The compounds according to the invention and their compositions according to the invention are moreover suitable for the control of harmful fungi in material protection and structural protection (e.g. wood, paper, dispersions for painting, fibers or fabric) and in storage protection. In wood and structural protection, the following harmful fungi in particular attract attention: Ascomycetes such as Ophiostoma spp., Ceratocystis spp., Aureobasidium pullulans, Sclerophoma spp., Chaetomium spp., Humicola spp., Petriella spp., Trichurus spp.; Basidiomycetes such as Coniophora spp., Coriolus spp., Gloeophyllum spp., Lentinus spp., Pleurotus spp., Poria spp., Serpula spp. and Tyromyces spp., Deuteromycetes such as Aspergillus spp., Cladosporium spp., Penicillium spp., Trichoderma spp., Alternaria spp., Paecilomyces spp. and Zygomycetes such as Mucor spp.; moreover the following yeast fungi in material protection: Candida spp. and Saccharomyces cerevisae.

The compounds according to the invention and their compositions according to the invention are suitable for improving plant health. Moreover, the invention relates to a method for improving plant health by treating the plants, the plant propagation material and/or the site at which the plants grow or are intended to grow with an effective amount of the compounds according to the invention and their according to the invention.

The term “plant health” comprises those states of a plant and/or its harvested material which are determined by various indicators individually or in combination, such as, for example, yield (for example increased biomass and/or increased content of utilizable ingredients), plant vitality (for example increased plant growth and/or greener leaves (“greening effect”)), quality (for example increased content or composition of certain ingredients) and tolerance to biotic and/or abiotic stress. The indicators mentioned here for a state of plant health may occur independently of one another or may influence each other.

Accordingly, the invention also provides the use of compounds according to the invention and/or agriculturally acceptable salts thereof for controlling phytopathogenic fungi.

The invention furthermore provides a method for controlling phytopathogenic fungi which comprises treating the fungi or the materials, plants, the soil or seed to be protected against fungal attack with an effective amount of a compound according to the invention and/or an agriculturally acceptable salt thereof.

The compounds according to the invention are used as such or in the form of a composition by treating the harmful fungi, their habitat or the plants to be protected from fungal attack, plant propagation materials, e.g. seed, the soil, surfaces, materials or spaces with a fungicidally active amount of the compounds according to the invention. Use can take place both before and after the infection of the plants, plant propagation materials, e.g. seed, the soil, the surfaces, materials or spaces by the fungi.

Plant propagation materials can be preventively treated together with or even before sowing or together with or even before transplanting with compounds according to the invention or with compositions according to the invention thereof.

Moreover, the invention relates to agents or agrochemical compositions comprising a solvent or solid carrier and at least one compound according to the invention, and the use of these compositions for the control of harmful fungi. A subject of this invention is also an agent or an agrochemical composition comprising at least one compound according to the invention and/or an agriculturally acceptable salt thereof for use in plant protection. An agent of this type customarily comprises at least one liquid or solid carrier.

The invention therefore also comprises agents or agrochemical compositions which comprise a solid or liquid carrier and a fungicidal compound according to the invention. The name “liquid carrier” is in this case used synonymously with solvent.

An agrochemical composition comprises a fungicidally active amount of a compound according to the invention. The expression “active amount” means an amount of the agrochemical composition or of the compound according to the invention that is adequate for the control of harmful fungi on crop plants or in material and buildings protection and does not lead to appreciable damage to the treated crop plants. Such an amount can vary within a wide range and is influenced by numerous factors, such as, for example, the harmful fungi to be controlled, the respective crop plant or materials treated, the climatic conditions and compounds.

The compounds according to the invention, their N-oxides and their salts can be converted into the types customary for agrochemical compositions, e.g. solutions, emulsions, suspensions, dusts, powders, pastes and granules. The type of composition depends on the respective intended use; it should in each case guarantee a fine and uniform dispersion of the compound according to the invention.

The term “agent” is used synonymously in this connection with the term “composition”, in particular “agrochemical composition”, and “formulation”.

Examples of types of composition here are suspensions (SC, OD, FS), pastes, pastilles, wettable powders or dusts (WP, SP, SS, WS, DP, DS) or granules (GR, FG, GG, MG), which can either be soluble in water or dispersible (wettable), and gels for the treatment of plant propagation materials such as seed (GF).

In general, the types of composition (e.g. SC, OD, FS, WG, SG, WP, SP, SS, WS, GF) are employed in dilute form. Types of compositions such as DP, DS, GR, FG, GG and MG are generally employed undiluted.

The agrochemical compositions are prepared in a known manner (see, for example, U.S. Pat. No. 3,060,084, EP-A 707 445 (for liquid concentrates), Browning, “Agglomeration”, Chemical Engineering, Dec. 4, 1967, 147-48, Perry's Chemical Engineer's Handbook, 4. ed., McGraw-Hill, New York, 1963, 8-57 and ff., WO 91/13546, U.S. Pat. No. 4,172,714, U.S. Pat. No. 4,144,050, U.S. Pat. No. 3,920,442, U.S. Pat. No. 5,180,587, U.S. Pat. No. 5,232,701, U.S. Pat. No. 5,208,030, GB 2,095,558, U.S. Pat. No. 3,299,566, Klingman: Weed Control as a Science (John Wiley & Sons, New York, 1961), Hance et al.: Weed Control Handbook (8th Ed., Blackwell Scientific Publications, Oxford, 1989) and Mollet, H. and Grubemann, A.: Formulation technology (Wiley VCH Verlag, Weinheim, 2001).

The agrochemical compositions can furthermore also comprise auxiliaries customary for plant protection agents, where the choice of the auxiliaries depends on the actual application form or the active compound.

Examples of suitable auxiliaries are solvents, solid carriers, surface-active substances (such as further solubilizers, protective colloids, wetting agents and adhesives), organic and inorganic thickeners, bactericides, frost protection agents, anti-foams, if appropriate colorants and adhesives (e.g. for seed treatment).

Suitable solvents are water, organic solvents such as mineral oil fractions of medium to high boiling point such as kerosene and diesel oil, furthermore coal tar oils and oils of plants or animal origin, aliphatic, cyclic and aromatic hydrocarbons, e.g. paraffins, tetrahydronaphthalene, alkylated naphthalenes and their derivatives, alkylated benzenes and their derivatives, alcohols such as methanol, ethanol, propanol, butanol and cyclohexanol, glycols, ketones such as cyclohexanone, gamma-butyrolactone, dimethylfatty acid amides, fatty acids and fatty acid esters and strongly polar solvents, e.g. amines such as N-methylpyrrolidone. In principle, solvent mixtures can also be used as well as mixtures of the abovementioned solvents and water.

Solid carriers are mineral earths such as silicic acids, silica gels, silicates, talc, kaolin, limestone, lime, chalk, bole, loess, clay, dolomite, diatomaceous earths, calcium sulfate and magnesium sulfate, magnesium oxide, ground plastics, fertilizers, such as ammonium sulfate, ammonium phosphate, ammonium nitrate, ureas and plant products such as cereal flour, treebark, wood and nutshell meal, cellulose powder or other solid carriers.

Suitable surface-active substances (adjuvants, wetting agents, adhesives, dispersants or emulsifiers) are the alkali metal, alkaline earth metal or ammonium salts of aromatic sulfonic acids, e.g. of lignosulfonic acid (Borresperse® types, Borregaard, Norway), phenolsulfonic acid, naphthalenesulfonic acid (Morwet® types, Akzo Nobel, USA) and dibutylnaphthalenesulfonic acid (Nekal® types, BASF, Germany), and also of fatty acids, alkyl- and alkylarylsulfonates, alkyl sulfates, lauryl ether sulfates and fatty alcohol sulfates, and also salts of sulfated hexa-, hepta- and octadecanols and/or fatty alcohol glycol ethers, condensation products of sulfonated naphthalene and its derivatives with formaldehyde, condensation products of naphthalene or of naphthalenesulfonic acids with phenol and formaldehyde, polyoxyethylene octylphenol ether, ethoxylated isooctyl, octyl or nonylphenol, alkylphenyl or tributylphenyl polyglycol ethers, alkylaryl polyether alcohols, isotridecyl alcohol, fatty alcohol-ethylene oxide condensates, ethoxylated castor oil, polyoxyethylene or polyoxypropylene alkyl ethers, lauryl alcohol polyglycol ether acetate, sorbitan esters, lignin-sulfite waste liquors and proteins, denatured proteins, polysaccharides (e.g. methylcellulose), hydrophobically modified starches, polyvinyl alcohol (Mowiol® types, Clariant, Switzerland), polycarboxylates (Sokalan® types, BASF, Germany), polyalkoxylates, polyvinylamine (Lupamin® types, BASF, Germany), polyethylenimine (Lupasol® types, BASF, Germany), polyvinylpyrrolidone and their copolymers.

Examples of thickeners (i.e. compounds that impart a modified flow behavior to the composition, i.e. high viscosity in the resting state and low viscosity in the stirred state) are polysaccharides and organic and inorganic layer minerals such as xanthan gum (Kelzan®, CP Kelco, USA), Rhodopol® 23 (Rhodia, France) or Veegum® (R.T. Vanderbilt, USA) or Attaclay® (Engelhard Corp., NJ, USA).

Bactericides can be added for the stabilization of the composition. Examples of bactericides are those based on dichlorophene and benzyl alcohol hemiformal (Proxel® from ICI or Acticide® RS from Thor Chemie and Kathon® MK from Rohm & Haas) and also isothiazolinone derivatives such as alkylisothiazolinones and benzisothiazolinones (Acticide® MBS from Thor Chemie).

Examples of suitable frost protection agents are ethylene glycol, propylene glycol, urea and glycerol.

Examples of antifoams are silicone emulsions (such as, for example, Silikon® SRE, Wacker, Germany or Rhodorsil®, Rhodia, France), long-chain alcohols, fatty acids, salts of fatty acids, fluoroorganic compounds and their mixtures.

Examples of colorants are both pigments that are sparingly soluble in water and dyes that are soluble in water. Examples which may be mentioned are the dyes and pigments known under the names Rhodamine B, C. I. Pigment Red 112 and C. I. Solvent Red 1, Pigment blue 15:4, Pigment blue 15:3, Pigment blue 15:2, Pigment blue 15:1, Pigment blue 80, Pigment yellow 1, Pigment yellow 13, Pigment red 48:2, Pigment red 48:1, Pigment red 57:1, Pigment red 53:1, Pigment orange 43, Pigment orange 34, Pigment orange 5, Pigment green 36, Pigment green 7, Pigment white 6, Pigment brown 25, Basic violet 10, Basic violet 49, Acid red 51, Acid red 52, Acid red 14, Acid blue 9, Acid yellow 23, Basic red 10, Basic red 108.

Examples of adhesives are polyvinylpyrrolidone, polyvinyl acetate, polyvinyl alcohol and cellulose ethers (Tylose®, Shin-Etsu, Japan).

Mineral oil fractions of medium to high boiling point, such as kerosene or diesel oil, furthermore coal tar oils and oils of plant or animal origin, aliphatic, cyclic and aromatic hydrocarbons, e.g. toluene, o-xylene, paraffin, tetrahydronaphthalene, alkylated naphthalenes or their derivatives, methanol, ethanol, propanol, butanol, cyclohexanol, cyclohexanone, isophorone, strongly polar solvents, e.g. dimethyl sulfoxide, N-methylpyrrolidone or water are suitable for the production of directly spreadable solutions, emulsions, pastes or oil dispersions.

Powders, broadcasting compositions and dusts can be prepared by mixing or joint grinding of the compounds I and, if present, further active compounds with at least one solid carrier.

Granules, e.g. coating, impregnated and homogeneous granules, can be prepared by binding the active compounds to at least one solid carrier. Solid carriers are, for example, mineral earths, such as silica gels, silicates, talc, kaolin, attaclay, limestone, lime, chalk, bole, loess, clay, dolomite, diatomaceous earths, calcium sulfate and magnesium sulfate, magnesium oxide, ground plastics, fertilizers such as ammonium sulfate, ammonium phosphate, ammonium nitrate, ureas and plant products, such as cereal meal, treebark, wood and nutshell meal, cellulose powder and other solid carriers.

Examples of types of composition are:

### 1. Compositions for Dilution in Water

i) Water-Soluble Concentrates (SL, LS)

10 parts by weight of the active compounds are dissolved with 90 parts by weight of water or a water-soluble solvent. Alternatively, wetting agents or other auxiliaries are added. On dilution in water the active compound dissolves. In this way a composition with a 10% by weight active compound content is obtained.

ii) Dispersible Concentrates (DC)

20 parts by weight of the active compounds are dissolved in 70 parts by weight of cyclohexanone with addition of 10 parts by weight of a dispersing agent, e.g. polyvinylpyrrolidone. On dilution in water a dispersion results. The active compound content is 20% by weight

iii) Emulsifiable Concentrates (EC)

15 parts by weight of the active compounds are dissolved in 75 parts by weight of xylene with addition of calcium dodecylbenzenesulfonate and castor oil ethoxylate (in each case 5 parts by weight). On dilution in water an emulsion results. The composition has a 15% by weight active compound content.

iv) Emulsions (EW, EO, ES)

25 parts by weight of the active compounds are dissolved in 35 parts by weight of xylene with addition of calcium dodecylbenzenesulfonate and castor oil ethoxylate (in each case 5 parts by weight). This mixture is added by means of an emulsifier (e.g. Ultra-Turrax) to 30 parts by weight of water and brought to a homogeneous emulsion. On dilution in water an emulsion results. The composition has an active compound content of 25% by weight.

v) Suspensions (SC, OD, FS)

20 parts by weight of the active compounds are comminuted with addition of 10 parts by weight of dispersing and wetting agents and 70 parts by weight of water or an organic solvent in a stirred ball mill to give a fine active compound suspension. On dilution in water a stable suspension of the active compound results. The active compound content in the composition is 20% by weight.

vi) Water-Dispersible and Water-Soluble Granules (WG, SG)

50 parts by weight of the active compounds are finely ground with addition of 50 parts by weight of dispersing and wetting agents and prepared as water-dispersible or water-soluble granules by means of technical equipment (e.g. extrusion, spray tower, fluidized bed). On dilution in water a stable dispersion or solution of the active compound results. The composition has an active compound content of 50% by weight.

vii) Water-Dispersible and Water-Soluble Powders (WP, SP, SS, WS)

75 parts by weight of the active compounds are ground with addition of 25 parts by weight of dispersing and wetting agents and silica gel in a rotor-stator mill. On dilution in water a stable dispersion or solution of the active compound results. The active compound content of the composition is 75% by weight.

viii) Gels (GF)

In a ball mill, 20 parts by weight of the active compounds, 10 parts by weight of dispersing agents, 1 part by weight of swelling agent (“gelling agent”) and 70 parts by weight of water or of an organic solvent are ground to give a fine suspension. On dilution with water a stable suspension with a 20% by weight active compound content results.

### 2. Types of Composition for Direct Application

ix) Dusts (DP, DS)

5 parts by weight of the active compounds are finely ground and intimately mixed with 95 parts by weight of finely divided kaolin. A dusting composition with a 5% by weight active compound content is obtained thereby.

x) Granules (GR, FG, GG, MG)

0.5 part by weight of the active compounds is finely ground and combined with 99.5 parts by weight of carriers. Customary processes here are extrusion, spray drying or the fluidized bed. Granules for direct application with a 0.5% by weight active compound content are obtained thereby.

xi) ULV Solutions (UL)

10 parts by weight of the active compounds are dissolved in 90 parts by weight of an organic solvent, e.g. xylene. A composition for direct application with a 10% by weight active compound content is obtained thereby.

The compositions of the compounds according to the invention generally comprise between 0.1 and 95% by weight, preferably between 0.1 and 90% by weight, preferably between 0.5 and 90% by weight, of active compound (compound according to the invention). The compounds I and II are in this case used in a purity of from 90% to 100%, preferably 95% to 100% (according to NMR spectrum).

For the treatment of plant propagation materials, in particular seed, use is usually made of water-soluble concentrates (LS), suspensions (FS), dustable powders (DS), water-dispersible and water-soluble powders (WS, SS), emulsions (ES), emulsifiable concentrates (EC) and gels (GF). These compositions can be applied to the propagation materials, in particular seed, in undiluted form or, preferably, diluted. In this case, the corresponding composition can be diluted 2-tuply to 10-tuply, so that from 0.01 to 60% by weight, preferably from 0.1 to 40%, by weight of active compound are present in the compositions to be used for the seed dressing. Application can be carried out prior to or during sowing. The treatment of plant propagation material, in particular the treatment of seed, is known to the person skilled in the art, and takes place by dusting, coating, pelleting, dipping or impregnation of the plant propagation material, the treatment preferably taking place by means of pelleting, coating and dusting or by furrow treatment, such that, for example, premature germination of the seed is prevented.

For the treatment of seed, suspensions are preferably used. Such compositions customarily comprise from 1 to 800 g/l of active compound, from 1 to 200 g/l of surfactants, from 0 to 200 g/l of frost protection agents, from 0 to 400 g/l of binders, from 0 to 200 g/l of colorants and solvents, preferably water.

The compounds can be used as such or in the form of their compositions, e.g. in the form of directly sprayable solutions, powders, suspensions, dispersions, emulsions, oil dispersions, pastes, dustable products, materials for broadcasting or granules, by means of spraying, atomizing, dusting, broadcasting, brushing, dipping or watering. The composition types depend entirely on the intended purposes; they should always ensure the finest possible distribution of the active compounds according to the invention.

Aqueous use forms can be prepared from emulsifiable concentrates, pastes or wettable powders (spray powders, oil dispersions) by addition of water. To prepare emulsions, pastes or oil dispersions, the substances can be homogenized in water, as such or dissolved in an oil or solvent, by means of wetting agents, tackifiers, dispersants or emulsifiers. However, it is also possible to prepare concentrates comprising active substance, wetting agent, tackifier, dispersant or emulsifier and possibly solvent or oil which are suitable for dilution with water.

The concentrations of active compound in the ready-for-use preparations can be varied within relatively wide ranges. In general, they are between 0.0001 and 10%, preferably between 0.01 and 1%.

The active compounds can also be used successfully in the ultra-low volume (ULV) process, it being possible to apply compositions with more than 95% by weight of active compound or even the active compound without additives.

When employed in crop protection, the application rates of the compounds according to the invention are, depending on the kind of effect desired, between 0.001 and 2.0 kg of active compound per ha, preferably between 0.005 and 2 kg per ha, preferably between 0.01 and 2.0 kg of active compound per ha, more preferably between 0.05 and 0.9 kg per ha, in particular between 0.1 and 0.75 kg per ha.

In the treatment of plant propagation materials e.g. seed, the amounts of active compound required are generally from 0.1 to 1000 g/100 kg of propagation material or seed, preferably 1 to 1000 g/100 kg, more preferably 1 to 100 g/100 kg, in particular 5 to 100 g/100 kg of propagation material or seed. Accordingly, the invention also provides seed comprising at least one compound according to the invention and/or an agriculturally acceptable salt thereof in an amount of from 1 to 1000 g per 100 kg.

When used in the protection of materials or stored products, the active compound application rate depends on the kind of application area and on the desired effect. Amounts typically applied in the protection of materials are, for example, from 0.001 g to 2 kg, preferably from 0.005 g to 1 kg, of active compound per cubic meter of treated material.

Oils of various types, wetting agents, adjuvants, herbicides, bactericides, further fungicides, and/or other pesticides can be added to the compounds according to the invention (active compounds) or the compositions comprising them, if appropriate also not until immediately before use (tank mix). These agents can be added to the compositions according to the invention in a weight ratio of 1:100 to 100:1, preferably of 1:10 to 10:1.

The following are particularly suitable as adjuvants in this context: organically modified polysiloxanes, for example Break Thru S 240®; alcohol alkoxylates, for example Atplus 245®, Atplus MBA 1303®, Plurafac LF 300® and Lutensol ON 30®; EO-PO block polymers, for example Pluronic RPE 2035® and Genapol B®; alcohol ethoxylates, for example Lutensol XP 80®; and sodium dioctylsulfosuccinate, for example Leophen RA®.

The compounds according to the invention or their compositions in the application form as fungicides can also be present together with other active compounds, for example with herbicides, insecticides, growth regulators, fungicides or else with fertilizers, as premix or if appropriate also not until immediately before use (tank mix). When mixing the compounds according to the invention or the compositions comprising them with one or more further active compounds, in particular fungicides, it is in many cases possible, for example, to widen the activity spectrum or to prevent the development of resistance. In many cases, synergistic effects are obtained.

Accordingly, the invention also provides a composition for crop protection which comprises a compound according to the invention, in particular a compound I or a compound II, and at least one further fungicidally, insecticidally and/or herbicidally active compound. According to one embodiment, the further active compound is a fungicidally active compound, in particular one selected from the list below. Here, the active compounds are preferably present in synergistic amounts.

A further subject matter of the invention relates to a composition comprising a compound according to the invention and/or an acid addition salt or metal salt thereof. As crop protection composition, this composition furthermore comprises at least one solid or liquid carrier. According to a further embodiment, the compositions mentioned may furthermore comprise at least one further fungicidally, insecticidally and/or herbicidally active compound. According to a further embodiment, the compositions comprise at least two further fungicidally active compounds, in particular two active compounds selected from the fungicides mentioned below.

The fungicides are preferably selected from the following groups:

Strobilurins, carboxamides, such as carboxanilides, carboxylic acid morpholides, benzamides, other carboxamides, azoles, such as triazoles, imidazoles, benzimidazoles, others, nitrogenous heterocyclyl compounds, such as pyridines, pyrimidines, pyrroles, morpholines, dicarboximides, other nitrogenous heterocyclyl compounds, thio- and dithiocarbamates, carbamates, guanidines, antibiotics, nitrophenyl derivatives, organometal compounds, sulfur-containing heterocyclyl compounds, organophosphorus compounds, organochlorine compounds, inorganic active compounds, other fungicides.

The following list of active compounds with which the compounds according to the invention can be applied together is meant to illustrate the possible combinations, but not to limit them:

### A) Strobilurins

azoxystrobin, dimoxystrobin, enestroburin, fluoxastrobin, kresoxim-methyl, methominostrobin, orysastrobin, picoxystrobin, pyraclostrobin, pyribencarb, trifloxystrobin, 2-(2-(6-(3-chloro-2-methylphenoxy)-5-fluoropyrimidin-4-yloxy)phenyl)-2-methoxyimino-N-methylacetamide, methyl 2-(ortho-((2,5-dimethylphenyloxymethylene)phenyl)-3-methoxyacrylate, methyl 3-methoxy-2-(2-(N-(4-methoxyphenyl)cyclopropanecarboximidoylsulfanylmethyl)phenyl)acrylate, 2-(2-(3-(2,6-dichlorophenyl)-1-methylallylideneaminooxymethyl)phenyl-2-methoxy-imino-N-methyl-acetamide;

### B) Carboxamides

carboxanilides: benalaxyl, benalaxyl-M, benodanil, bixafen, boscalid, carboxin, fenfuram, fenhexamid, flutolanil, furametpyr, isopyrazam, isotianil, kiralaxyl, mepronil, metalaxyl, metaloxyl-M (mefenoxam), ofurace, oxadixyl, oxycarboxin, penthiopyrad, sedaxane, tecloftalam, thifluzamide, tiadinil, 2-amino-4-methylthiazole-5-carboxanilide, 2-chloro-N-(1,1,3-trimethylindan-4-yl)-nicotinamide, N-(3′,4′-dichloro-5-fluorobiphenyl-2-yl)-3-difluoromethyl-1-methyl-1H-pyrazole-4-carboxamide, [2-(1,3-dimethylbutyl)phenyl]-5-fluoro-1,3-dimethyl-1H-pyrazole-4-carboxamide, N-(4′-chloro-3′,5-difluorobiphenyl-2-yl)-3-difluoromethyl-1-methyl-1H-pyrazole-4-carboxamide, N-(4′-chloro-3′,5-difluorobiphenyl-2-yl)-3-trifluoromethyl-1-methyl-1H-pyrazole-4-carboxamide, N-(3′,4′-dichloro-4-fluorobiphenyl-2-yl)-3-trifluoromethyl-1-methyl-1H-pyrazole-4-carboxamide, N-(3′,5-difluoro-4′-methylbiphenyl-2-yl)-3-difluoromethyl-1-methyl-1H-pyrazole-4-carboxamide, N-(3′,5-difluoro-4′-methylbiphenyl-2-yl)-3-trifluoromethyl-1-methyl-1H-pyrazole-4-carboxamide, N-(2-bicyclopropyl-2-ylphenyl)-3-difluoromethyl-1-methyl-1H-pyrazole-4-carboxamide, N-(cis-2-bicyclopropyl-2-ylphenyl)-3-difluoromethyl-1-methyl-1H-pyrazole-4-carboxamide, N-(trans-2-bicyclopropyl-2-ylphenyl)-3-difluoromethyl-1-methyl-1H-pyrazole-4-carboxamide, N-(4′-bromobiphenyl-2-yl)-4-difluoromethyl-2-methylthiazole-5-carboxamide, N-(4′-trifluoromethylbiphenyl-2-yl)-4-difluoromethyl-2-methylthiazole-5-carboxamide, N-(4′-chloro-3′-fluorobiphenyl-2-yl)-4-difluoromethyl-2-methylthiazole-5-carboxamide, 3,4-dichloro-N-(2-cyanophenyl)isothiazole-5-carboxamide, N-(2′,4′-difluorobiphenyl-2-yl)-1-methyl-3-trifluoromethyl-1H-pyrazole-4-carboxamide, N-(2′,4′-dichlorobiphenyl-2-yl)-1-methyl-3-trifluoromethyl-1H-pyrazole-4-carboxamide, N-(2′,4′-difluorobiphenyl-2-yl)-3-difluoromethyl-1-methyl-1H-pyrazole-4-carboxamide, N-(2′,4′-dichlorobiphenyl-2-yl)-3-difluoromethyl-1-methyl-1H-pyrazole-4-carboxamide, N-(2′,5′-difluorobiphenyl-2-yl)-1-methyl-3-trifluoromethyl-1H-pyrazole-4-carboxamide, N-(2′,5′-dichlorobiphenyl-2-yl)-1-methyl-3-trifluoromethyl-1H-pyrazole-4-carboxamide, N-(2′,5′-difluorobiphenyl-2-yl)-3-difluoromethyl-1-methyl-1H-pyrazole-4-carboxamide, N-(2′,5′-dichlorobiphenyl-2-yl)-3-difluoromethyl-1-methyl-1H-pyrazole-4-carboxamide, N-(3′,5′-difluorobiphenyl-2-yl)-1-methyl-3-trifluoromethyl-1H-pyrazole-4-carboxamide, N-(3′,5′-dichlorobiphenyl-2-yl)-1-methyl-3-trifluoromethyl-1H-pyrazole-4-carboxamide, N-(3′,5′-difluorobiphenyl-2-yl)-3-difluoromethyl-1-methyl-1H-pyrazole-4-carboxamide, N-(3′,5′-dichlorobiphenyl-2-yl)-3-difluoromethyl-1-methyl-1H-pyrazole-4-carboxamide, N-(3′-fluorobiphenyl-2-yl)-1-methyl-3-trifluoromethyl-1H-pyrazole-4-carboxamide, N-(3′-chlorobiphenyl-2-yl)-1-methyl-3-trifluoromethyl-1H-pyrazole-4-carboxamide, N-(3′-fluorobiphenyl-2-yl)-3-difluoromethyl-1-methyl-1H-pyrazole-4-carboxamide, N-(3′-chlorobiphenyl-2-yl)-3-difluoromethyl-1-methyl-1H-pyrazole-4-carboxamide, N-(2′-fluorobiphenyl-2-yl)-1-methyl-3-trifluoromethyl-1H-pyrazole-4-carboxamide, N-(2′-chlorobiphenyl-2-yl)-1-methyl-3-trifluoromethyl-1H-pyrazole-4-carboxamide, N-(2′-fluorobiphenyl-2-yl)-3-difluoromethyl-1-methyl-1H-pyrazole-4-carboxamide, N-(2′-chlorobiphenyl-2-yl)-3-difluoromethyl-1-methyl-1H-pyrazole-4-carboxamide, N-(2′-fluoro-4′-chloro-5′-methylbiphenyl-2-yl)-1-methyl-3-trifluoromethyl-1H-pyrazole-4-carboxamide, N-(3′,4′,5′-trifluorobiphenyl-2-yl)-1-methyl-3-trifluoromethyl-1H-pyrazole-4-carboxamide, N-(3′,4′,5′-trifluorobiphenyl-2-yl)-1-methyl-3-difluoromethyl-1H-pyrazole-4-carboxamide, N-(2′,4′,5′-tri-fluorobiphenyl-2-yl)-1-methyl-3-difluoromethyl-1H-pyrazole-4-carboxamide, N-(3′,4′,5′-trifluorobiphenyl-2-yl)-3-chlorofluoromethyl-1-methyl-1H-pyrazole-4-carboxamide, N-[2-(1,1,2,3,3,3-hexafluoropropoxy)phenyl]-1-methyl-3-trifluoromethyl-1H-pyrazole-4-carboxamide, N-[2-(1,1,2,3,3,3-hexafluoropropoxy)-phenyl]-3-difluoromethyl-1-methyl-1H-pyrazole-4-carboxamide, N-[2-(2-chloro-1,1,2-trifluoroethoxy)phenyl]-1-methyl-3-trifluoromethyl-1H-pyrazole-4-carboxamide, N-[2-(2-chloro-1,1,2-trifluoroethoxy)phenyl]-3-difluoromethyl-1-methyl-1H-pyrazole-4-carboxamide, N-[2-(1,1,2,2-tetrafluoroethoxy)phenyl]-3-difluoromethyl-1-methyl-1H-pyrazole-4-carboxamide, N-[2-(1,1,2,2-tetrafluoroethoxy)phenyl]-1-methyl-3-trifluoromethyl-1H-pyrazole-4-carboxamide, N-(4′-(trifluoromethylthio)biphenyl-2-yl)-3-difluoromethyl-1-methyl-1H-pyrazole-4-carboxamide, N-(4′-(trifluoromethylthio)biphenyl-2-yl)-1-methyl-3-trifluoromethyl-1H-pyrazole-4-carboxamide, [2-(1,2-dimethylpropyl)phenyl]-5-fluoro-1,3-dimethyl-1H-pyrazole-4-carboxamide, N-(2-{4-[3-(4-chlorophenyl)prop-2-ynyloxy]-3-methoxyphenyl}ethyl)-2-methanesulfonylamino-3-methylbutyramide, N-(2-{4-[3-(4-chlorophenyl)prop-2-ynyloxy]-3-methoxyphenyl}ethyl)-2-ethanesulfonylamino-3-methylbutyramide, N-(3′,4′-dichloro-5-fluorobiphenyl-2-yl)-3-trifluoromethyl-1-methyl-1H-pyrazole-4-carboxamide, N-(2-(1,3,3-trimethylbutyl)phenyl)-1,3-dimethyl-5-fluoro-1H-pyrazole-4-carboxamide, N-[1,2,3,4-tetrahydro-9-(1-methylethyl)-1,4-methanonaphthalen-5-yl]-3-(difluoromethyl)-1-methyl-1H-pyrazole-4-carboxamide, N′-(4-(4-chloro-3-trifluoromethylphenoxy)-2,5-dimethylphenyl)-N-ethyl-N-methylformamidine, N′-(4-(4-fluoro-3-trifluoromethylphenoxy)-2,5-dimethylphenyl)-N-ethyl-N-methylformamidine, N′-(2-methyl-5-trifluoromethyl-4-(3-trimethylsilanylpropoxy)-phenyl)-N-ethyl-N-methylformamidine and N′-(5-difluoromethyl-2-methyl-4-(3-trimethylsilanylpropoxy)phenyl)-N-ethyl-N-methylformamidine;
carboxylic acid morpholides: dimethomorph, flumorph, pyrimorph;
benzamides: flumetover, fluopicolide, fluopyram, zoxamide, N-(3-ethyl-3,5,5-trimethylcyclohexyl)-3-formylamino-2-hydroxybenzamide;
other carboxamides: carpropamid, diclocymet, mandipropamid, oxytetracyclin, silthiofam, N-(6-methoxypyridin-3-yl)cyclopropanecarboxamide;

### C) Azoles

triazoles: azaconazole, bitertanol, bromuconazole, cyproconazole, difenoconazole, diniconazole, diniconazole-M, epoxiconazole, fenbuconazole, fluquinconazole, flusilazole, flutriafol, hexaconazole, imibenconazole, ipconazole, metconazole, myclobutanil, oxpoconazole, paclobutrazole, penconazole, propiconazole, prothioconazole, simeconazole, tebuconazole, tetraconazole, triadimefon, triadimenol, triticonazole, uniconazole, 1-(4-chlorophenyl)-2-([1,2,4]triazol-1-yl)-cycloheptanol;
imidazoles: cyazofamid, imazalil, imazalil-sulfate, pefurazoate, prochloraz, triflumizole;
benzimidazoles: benomyl, carbendazim, fuberidazole, thiabendazole;
others: ethaboxam, etridiazole, hymexazole, 1-(4-chlorophenyl)-1-(propyn-2-yloxy)-3-(4-(3,4-dimethoxyphenyl)isoxazol-5-yl)propan-2-one, 2-(4-chlorophenyl)-N-[4-(3,4-dimethoxyphenyl)isoxazol-5-yl]-2-prop-2-ynyloxyacetamide;

### D) Nitrogenous Heterocyclyl Compounds

pyridines: fluazinam, pyrifenox, 3-[5-(4-chlorophenyl)-2,3-dimethylisoxazolidin-3-yl]-pyridine, 3-[5-(4-methylphenyl)-2,3-dimethylisoxazolidin-3-yl]pyridine, 2,3,5,6-tetrachloro-4-methanesulfonylpyridine, 3,4,5-trichloropyridine-2,6-dicarbonitrile, N-(1-(5-bromo-3-chloropyridin-2-yl)ethyl)-2,4-dichloronicotinamide, N-((5-bromo-3-chloropyridin-2-yl)methyl)-2,4-dichloronicotinamide;
pyrimidines: bupirimate, cyprodinil, diflumetorim, fenarimol, ferimzone, mepanipyrim, nitrapyrin, nuarimol, pyrimethanil;
piperazines: triforine;
pyrroles: fludioxonil, fenpiclonil;
morpholines: aldimorph, dodemorph, dodemorph-acetate, fenpropimorph, tridemorph;
piperidines: fenpropidin;
dicarboximides: fluoroimide, iprodione, procymidone, vinclozolin;
nonaromatic 5-ring heterocycles: famoxadone, fenamidone, flutianil, octhilinone, probenazole, S-allyl 5-amino-2-isopropyl-3-oxo-4-orthotolyl-2,3-dihydropyrazole-1-thiocarboxylate;
others: acibenzolar-S-methyl, amisulbrom, anilazine, blasticidin-S, captafol, captan, quinomethionate, dazomet, debacarb, diclomezine, difenzoquat, difenzoquat-methyl sulfate, fenoxanil, folpet, oxolinic acid, piperalin, proquinazid, pyroquilon, quinoxyfen, triazoxide, tricyclazole, 5-chloro-7-(4-methylpiperidin-1-yl)-6-(2,4,6-trifluorophenyl)-[1,2,4]-triazolo[1,5-a]pyrimidine, 6-(4-tert-butylphenyl)-5-methyl-[1,2,4]triazolo[1,5-a]pyrimidin-7-ylamine, 5-methyl-6-(3,5,5-trimethylhexyl)-[1,2,4]triazolo[1,5-a]pyrimidin-7-ylamine, 5-methyl-6-octyl-[1,2,4]triazolo[1,5-a]pyrimidin-7-ylamine, 6-methyl-5-octyl-[1,2,4]triazolo[1,5-a]pyrimidin-7-ylamine, 6-ethyl-5-octyl-[1,2,4]-triazolo[1,5-a]pyrimidin-7-ylamine, 5-ethyl-6-octyl-[1,2,4]triazolo[1,5-a]pyrimidin-7-ylamine, 5-ethyl-6-(3,5,5-trimethylhexyl)-[1,2,4]triazolo[1,5-a]pyrimidin-7-ylamine, 6-octyl-5-propyl-[1,2,4]triazolo[1,5-a]pyrimidin-7-ylamine, 5-methoxymethyl-6-octyl-[1,2,4]triazolo[1,5-a]pyrimidin-7-ylamine, 6-octyl-5-trifluoromethyl[1,2,4]triazolo[1,5-a]pyrimidin-7-ylamine, 5-trifluoromethyl-6-(3,5,5-trimethylhexyl)-[1,2,4]-triazolo[1,5-a]pyrimidin-7-ylamine, 2-butoxy-6-iodo-3-propylchromen-4-one, 5-chloro-1-(4,6-dimethoxypyrimidin-2-yl)-2-methyl-1H-benzoimidazole, 6-(3,4-dichlorophenyl)-5-methyl-[1,2,4]triazolo-[1,5-a]pyrimidin-7-ylamine;

### E) Carbamates and Dithiocarbamates

thio- and dithiocarbamates: ferbam, mancozeb, maneb, metam, methasulfocarb, metiram, propineb, thiram, zineb, ziram;
carbamates: diethofencarb, benthiavalicarb, iprovalicarb, propamocarb, propamocarb hydrochloride, valiphenal, (4-fluorophenyl) N-(1-(1-(4-cyanophenyl)-ethanesulfonyl)but-2-yl)carbamate;

### F) Other Fungicides

guanidines: dodine, dodine-free base, guazatine, guazatine-acetate, iminoctadine, iminoctadine-triacetate, iminoctadine-tris(albesilate);
antibiotics: kasugamycin, kasugamycin-hydrochloride hydrate, polyoxins, streptomycin, validamycin A;
nitrophenyl derivatives:
   binapacryl, dicloran, dinobuton, dinocap, nitrothal-isopropyl, tecnazen;
organometallic compounds: fentin salts such as, for example, fentin-acetate, fentin-chloride, fentin-hydroxide;
sulfur-containing heterocyclyl compounds: isoprothiolane, dithianon;
organophosphorus compounds: edifenphos, fosetyl, fosetyl-aluminum, iprobenfos, pyrazophos, tolclofos-methyl;
organochlorine compounds: chlorothalonil, dichlofluanid, dichlorophen, flusulfamide, hexachlorobenzene, pencycuron, pentachlorophenol and salts thereof, phthalide, quintozene, thiophanate-methyl, tolylfluanid, N-(4-chloro-2-nitrophenyl)-N-ethyl-4-methylbenzenesulfonamide;
inorganic active compounds: phosphorous acid and salts thereof, sulfur, Bordeaux mixture, copper salts such as, for example, copper acetate, copper hydroxide, copper oxychloride, basic copper sulfate;
others: biphenyl, bronopol, cyflufenamid, cymoxanil, diphenylamine, metrafenone, mildiomycin, oxine-copper, prohexadione-calcium, spiroxamine, tolyifluanid, N-(cyclopropylmethoxyimino-(6-difluoromethoxy-2,3-difluorophenyl)methyl)-2-phenyl acetamide, N′-(4-(4-chloro-3-trifluoromethylphenoxy)-2,5-dimethylphenyl)-N-ethyl-N-methylformamidine, N′-(4-(4-fluoro-3-trifluoromethylphenoxy)-2,5-dimethylphenyl)-N-ethyl-N-methylformamidine, N′-(2-methyl-5-trifluoromethyl-4-(3-trimethylsilanyl-propoxy)phenyl)-N-ethyl-N-methylformamidine, N′-(5-difluoromethyl-2-methyl-4-(3-trimethylsilanylpropoxy)phenyl)-N-ethyl-N-methylformamidine, 2-{1-[2-(5-methyl-3-trifluoromethylpyrazol-1-yl)acetyl]piperidin-4-yl}thiazole-4-carboxylic acid methyl-(1,2,3,4-tetrahydronaphthalen-1-yl)amide, 2-{1-[2-(5-methyl-3-trifluoromethylpyrazol-1-yl)acetyl]piperidin-4-yl}thiazole-4-carboxylic acid methyl-(R)-1,2,3,4-tetrahydronaphthalen-1-yl-amide, 6-tert-butyl-8-fluoro-2,3-dimethylquinolin-4-yl acetate, 6-tert-butyl-8-fluoro-2,3-dimethylquinolin-4-yl methoxyacetate;

### G) Growth Regulators

abscisic acid, amidochlor, ancymidol, 6-benzylaminopurine, brassinolide, butralin, chlormequat (chlormequat chloride), choline chloride, cyclanilide, daminozide, dikegulac, dimethipin, 2,6-dimethylpuridine, ethephon, flumetralin, flurprimidol, fluthiacet, forchlorfenuron, gibberellic acid, inabenfide, indole-3-acetic acid, maleic hydrazide, mefluidide, mepiquat (mepiquat chloride), metconazole, naphthaleneacetic acid, N-6-benzyladenine, paclobutrazole, prohexadione (prohexadione-calcium), prohydrojasmone, thidiazuron, triapenthenol, tributyl phosphorotrithioate, 2,3,5-triiodbenzoic acid, trinexapac-ethyl and uniconazole;

### H) Herbicides

acetamides: acetochlor, alachlor, butachlor, dimethachlor, dimethenamid, flufenacet, mefenacet, metolachlor, metazachlor, napropamide, naproanilide, pethoxamid, pretilachlor, propachior, thenylchlor;
amino acid analogs: bilanafos, glyphosate, glufosinate, sulfosate;
aryloxyphenoxypropionates: clodinafop, cyhalofop-butyl, fenoxaprop, fluazifop, haloxyfop, metamifop, propaquizafop, quizalofop, quizalofop-P-tefuryl;
bipyridyls: diquat, paraquat;
carbamates and thiocarbamates: asulam, butylate, carbetamide, desmedipham, dimepiperate, eptam (EPTC), esprocarb, molinate, orbencarb, phenmedipham, prosulfocarb, pyributicarb, thiobencarb, triallate;
cyclohexanediones: butroxydim, clethodim, cycloxydim, profoxydim, sethoxydim, tepraloxydim, tralkoxydim;
dinitroanilines: benfluralin, ethalfluralin, oryzalin, pendimethalin, prodiamine, trifluralin;
diphenyl ethers: acifluorfen, aclonifen, bifenox, diclofop, ethoxyfen, fomesafen, lactofen, oxyfluorfen;
hydroxybenzonitriles: bromoxynil, dichlobenil, ioxynil;
imidazolinones: imazamethabenz, imazamox, imazapic, imazapyr, imazaquin, imazethapyr;
phenoxyacetic acids: clomeprop, 2,4-dichlorophenoxyacetic acid (2,4-D), 2,4-DB, dichlorprop, MCPA, MCPA-thioethyl, MCPB, mecoprop;
pyrazines: chloridazon, flufenpyr-ethyl, fluthiacet, norflurazon, pyridate;
pyridines: aminopyralid, clopyralid, diflufenican, dithiopyr, fluridone, fluoroxypyr, picloram, picolinafen, thiazopyr;
sulfonylureas: amidosulfuron, azimsulfuron, bensulfuron, chlorimuron-ethyl, chlorsulfuron, cinosulfuron, cyclosulfamuron, ethoxysulfuron, flazasulfuron, fluce-tosulfuron, flupyrsulfuron, foramsulfuron, halosulfuron, imazosulfuron, iodosulfuron, mesosulfuron, metsulfuron-methyl, nicosulfuron, oxasulfuron, primisulfuron, prosul-furon, pyrazosulfuron, rimsulfuron, sulfometuron, sulfosulfuron, thifensulfuron, tria-sulfuron, tribenuron, trifloxysulfuron, triflusulfuron, tritosulfuron, 1-((2-chloro-6-propylimidazo[1,2-b]pyridazin-3-yl)sulfonyl)-3-(4,6-dimethoxypyrimidin-2-yl)urea;
triazines: ametryn, atrazine, cyanazine, dimethametryn, ethiozine, hexazinone, metamitron, metribuzin, prometryn, simazine, terbuthylazine, terbutryn, triaziflam;
ureas: chlorotoluron, daimuron, diuron, fluometuron, isoproturon, linuron, methabenzthiazuron, tebuthiuron;
other inhibitors of acetolactate synthase: bispyribac-sodium, cloransulam-methyl, diclosulam, florasulam, flucarbazone, flumetsulam, metosulam, ortho-sulfamuron, penoxsulam, propoxycarbazone, pyribambenz-propyl, pyribenzoxim, pyriftalid, pyriminobac-methyl, pyrimisulfan, pyrithiobac, pyroxasulfone, pyroxsulam;
others: amicarbazone, aminotriazole, anilofos, beflubutamid, benazolin, bencarbazone, benfluresate, benzofenap, bentazone, benzobicyclon, bromacil, bromobutide, butafenacil, butamifos, cafenstrole, carfentrazone, cinidon-ethyl, chlorthal, cinmethylin, clomazone, cumyluron, cyprosulfamide, dicamba, difenzo-quat, diflufenzopyr, Drechslera monoceras, endothal, ethofumesate, etobenzanid, fentrazamide, flumiclorac-pentyl, flumioxazin, flupoxam, fluorochloridone, flurtamon, indanofan, isoxaben, isoxaflutol, lenacil, propanil, propyzamide, quinclorac, quinmerac, mesotrione, methylarsenic acid, naptalam, oxadiargyl, oxadiazone, oxaziclomefon, pentoxazone, pinoxaden, pyraclonil, pyraflufen-ethyl, pyrasulfotol, pyrazoxyfen, pyrazolynate, quinoclamine, saflufenacil, sulcotrione, sulfentrazone, terbacil, tefuryltrione, tembotrione, thiencarbazone, topramezon, 4-hydroxy-3-[2-(2-methoxyethoxymethyl)-6-trifluoromethylpyridine-3-carbonyl]bicyclo[3.2.1]oct-3-en-2-one, ethyl (3-[2-chloro-4-fluoro-5-(3-methyl-2,6-dioxo-4-trifluoromethyl-3,6-dihydro-2H-pyrimidin-1-yl)phenoxy]pyridin-2-yloxy)acetate, methyl 6-amino-5-chloro-2-cyclopropyl-pyrimidin-4-carboxylate, 6-chloro-3-(2-cyclopropyl-6-methylphenoxy)-pyridazin-4-ol, 4-amino-3-chloro-6-(4-chlorophenyl)-5-fluoropyridin-2-carboxylic acid, methyl 4-amino-3-chloro-6-(4-chloro-2-fluoro-3-methoxyphenyl)pyridine-2-carboxylate and methyl-4-amino-3-chloro-6-(4-chloro-3-dimethylamino-2-fluorophenyl)pyridine-2-carboxylate;

### I) Insecticides

organo(thio)phosphates: acephate, azamethiphos, azinphos-methyl, chlorpyrifos, chlorpyrifos-methyl, chlorfenvinphos, diazinone, dichlorvos, dicrotophos, dimethoate, disulfoton, ethion, fenitrothion, fenthion, isoxathion, malathion, methamidophos, methidathion, methyl-parathion, mevinphos, monocrotophos, oxydemeton-methyl, paraoxon, parathion, phenthoate, phosalone, phosmet, phosphamidon, phorate, phoxim, pirimiphos-methyl, profenofos, prothiofos, sulprophos, tetrachlorvinphos, terbufos, triazophos, trichlorfon;
carbamates: alanycarb, aldicarb, bendiocarb, benfuracarb, carbaryl, carbofuran, carbosulfan, fenoxycarb, furathiocarb, methiocarb, methomyl, oxamyl, pirimicarb, propoxur, thiodicarb, triazamate;
pyrethroids: allethrin, bifenthrin, cyfluthrin, cyhalothrin, cyphenothrin, cypermethrin, alpha-cypermethrin, beta-cypermethrin, zeta-cypermethrin, deltamethrin, esfenvalerate, etofenprox, fenpropathrin, fenvalerate, imiprothrin, lambda-cyhalothrin, permethrin, prallethrin, pyrethrin I and II, resmethrin, silafluofen, tau-fluvalinate, tefluthrin, tetramethrin, tralomethrin, transfluthrin, profluthrin, dimefluthrin,
inhibitors of insect growth: a) chitin synthesis inhibitors: benzoylureas: chlorfluazuron, cyromazine, diflubenzuron, flucycloxuron, flufenoxuron, hexaflumuron, lufenuron, novaluron, teflubenzuron, triflumuron; buprofezin, diofenolan, hexythiazox, etoxazole, clofentazine; b) ecdysone antagonists: halofenozide, methoxyfenozide, tebufenozide, azadirachtin; c) juvenoids: pyriproxyfen, methoprene, fenoxycarb; d) lipid biosynthesis inhibitors: spirodiclofen, spiromesifen, spirotetramate;
nicotine receptor agonists/antagonists: clothianidin, dinotefuran, imidacloprid, thiamethoxam, nitenpyram, acetamiprid, thiacloprid, 1-(2-chlorothiazol-5-ylmethyl)-2-nitrimino-3,5-dimethyl-[1,3,5]triazinane;
GABA antagonists: endosulfan, ethiprol, fipronil, vaniliprol, pyrafluprol, pyriprol, 5-amino-1-(2,6-dichloro-4-methylphenyl)-4-sulfinamoyl-1H-pyrazole-3-thio-carboxamide;
macrocyclic lactones: abamectin, emamectin, milbemectin, lepimectin, spinosad, spinetoram;
mitochondrial electron transport chain inhibitor (METI) I acaricides: fenazaquin, pyridaben, tebufenpyrad, tolfenpyrad, flufenerim;
METI II and III substances: acequinocyl, fluacyprim, hydramethylnon;
decouplers: chlorfenapyr;
inhibitors of oxidative phosphorylation: cyhexatin, diafenthiuron, fenbutatin oxide, propargite;
inhibitors of ecdysis of insects: cyromazine;
inhibitors of mixed function oxidases: piperonyl butoxide;
sodium channel blockers: indoxacarb, metaflumizone;
others: benclothiaz, bifenazate, cartap, flonicamid, pyridalyl, pymetrozine, sulfur, thiocyclam, flubendiamide, chiorantraniliprol, cyazypyr (HGW86); cyenopyrafen, flupyrazofos, cyflumetofen, amidoflumet, imicyafos, bistrifluoron and pyrifluquinazon.

The present invention in particular also relates to fungicidal compositions that comprise at least one compound of the general formula I and at least one further plant protection active compound, e.g. selected from the active compounds of the aforementioned groups A) to I), in particular at least one fungicidal active compound, in particular selected from the aforementioned groups A) to F), and if appropriate one or more agriculturally suitable carriers. With respect to reduction of the application amounts, these mixtures are of interest, since many, in the case of a decreased total amount of applied active compound, show an improved action against harmful fungi, in particular for certain indications. By means of a simultaneous joint or separate application of compound(s) I with at least one active compound of the groups A) to I), the fungicidal activity can be increased superadditively.

Joint application within the meaning of this application means that the at least one compound I and the at least one further active compound are present simultaneously at the site of action (i.e. the plant-injurious fungi to be controlled and their habitat, such as diseased plants, plant propagation materials, in particular seed, soils, materials or spaces, and the plants, plant propagation materials, in particular seed, soils, materials or spaces to be protected from fungal attack) in an amount adequate for effective control of fungal growth. This can be achieved by simultaneously applying the compounds I and at least one further active compound together in a common active compound preparation or in at least two separate active compound preparations simultaneously or by applying the active compounds successively at the site of action, where the separation in time of the individual active compound applications is chosen such that the active compound applied first is present at the site of action in adequate amount at the time of application of the further active compound/compounds. The time sequence of the application of the active compounds is of minor importance.

In binary mixtures, i.e. compositions according to the invention that comprise a compound I and a further active compound, e.g. an active compound from the groups A) to I), the weight ratio of compound I to the further active compound is the weight ratio of compound I to the 1st further active compound depends on the properties of the respective active compounds, customarily it is in the range from 1:100 to 100:1, often in the range from 1:50 to 50:1, preferably in the range from 1:20 to 20:1, particularly preferably in the range from 1:10 to 10:1, in particular in the range from 1:3 to 3:1.

In one embodiment of the invention, the kits can comprise one or more, even all, components that can be used for the preparation of an agrochemical composition according to the invention. For example, these kits can comprise one or more fungicide components(s) and/or an adjuvant component and/or an insecticide component and/or a growth regulator component and/or a herbicide. One or more components can be combined with one another or be present in preformulated form. In the embodiments in which more than two components are prepared in one kit, the components can be combined with one another and be present packaged in an individual container such as a jar, bottle, can, pouch, bag or canister. In other embodiments, two or more components of a kit can be packed separately, i.e. not preformulated or mixed. Kits can comprise one or more separate containers such as jars, bottles, cans, pouches, bags or canisters, where each container comprises a separate component of the agrochemical composition. The components of the composition according to the invention can be packed individually or already mixed or as parts according to the kit principle (‘kit of parts’) and can be reused. In both forms, one component can be used separately or together with the further components or as a constituent of a ‘kit of parts’ for the preparation of the mixture according to the invention.

The user customarily uses the composition according to the invention for application in a predosing device, in a backpack sprayer, in a spray tank or in a crop duster. Here, the agrochemical composition is brought to the desired application concentration with water and/or buffer, further auxiliaries being added if appropriate, and thus the ready-to-use spray liquor or the agrochemical composition according to the invention is obtained. Customarily, 50 to 500 liters of the ready-to-use spray liquor are applied per hectare of agricultural area, preferably 100 to 400 liters.

According to one embodiment, the user can mix individual components such as, for example, parts of a kit or of a mixture of the composition according to the invention itself in the spray tank and if appropriate add further auxiliaries (tankmix).

In a further embodiment, the user can mix both individual components of the composition according to the invention and partially premixed components, for example components comprising compounds I and/or active compounds from the groups A) to I), in the spray tank and if appropriate add further auxiliaries (tankmix).

In a further embodiment, the user can use both individual components of the composition according to the invention and partially premixed components, for example components comprising compounds I and/or active compounds from the groups A) to I), together (e.g. as a tankmix) or successively.

Preferred are compositions of a compound I (component I) with at least one active compound from the group group A) (component 2) of the strobilurins and particularly selected from azoxystrobin, dimoxystrobin, fluoxastrobin, kresoxim-methyl, orysastrobin, picoxystrobin, pyraclostrobin and trifloxystrobin.

Also preferred are compositions of a compound I (component I) with at least one active compound selected from the group B) (component 2) of the carboxamides and particularly selected from bixafen, boscalid, sedaxane, fenhexamid, metalaxyl, mefenoxam, ofurace, dimethomorph, flumorph, fluopicolide (picobenzamid), zoxamid, carpropamid and mandipropamid.

Also preferred are compositions of a compound I (component I) with at least one active compound selected from the group C) (component 2) of the azoles and particularly selected from cyproconazole, difenoconazole, epoxiconazole, fluquinconazole, flusilazol, flutriafol, metconazole, myclobutanil, penconazole, propiconazole, prothioconazole, triadimefon, triadimenol, tebuconazole, tetraconazole, triticonazole, prochloraz, cyazofamid, benomyl, carbendazim and ethaboxam.

Also preferred are compositions of a compound I (component I) with at least one active compound selected from the group D) (component 2) of the nitrogenous heterocyclyl compounds and particularly selected from fluazinam, cyprodinil, fenarimol, mepanipyrim, pyrimethanil, triforin, fludioxonil, fodemorph, fenpropimorph, tridemorph, fenpropidin, iprodione, vinclozoline, famoxadone, fenamidone, probenazole, proquinazid, acibenzolar-S-methyl, captafol, folpet, fenoxanil and quinoxyfen.

Also preferred are compositions of a compound I (component I) with at least one active compound selected from the group E) (component 2) of the carbamates and particularly selected from mancozeb, metiram, propineb, thiram, iprovalicarb, benthiavalicarb and propamocarb.

Also preferred are compositions of a compound I (component I) with at least one active compound selected from the fungicides of the group F) (component 2) and particularly selected from dithianon, fentin salts, such as fentin acetate, fosetyl, fosetyl-aluminum, H₃PO₃ and its salts, chlorothalonil, dichlofluanid, thiophanate-methyl, copper acetate, copper hydroxide, copper oxychloride, copper sulfate, sulfur, cymoxanil, metrafenon, spiroxamine and 5-chloro-7-(4-methylpiperidin-1-yl)-6-(2,4,6-trifluorophenyl)-[1,2,4]-triazolo[1,5-a]pyrimidine.

The present invention further relates, accordingly, to compositions of a compound I (component 1) with a further active compound (component 2), the latter being selected from lines C-1 to C-416 in the “component 2” column of Table C. A further embodiment of the invention relates to the compositions C-1 to C-416 that are listed in Table C, each line of Table C corresponding to an agrochemical composition comprising a compound of the formula I (component 1), which is preferably one of the compounds described herein as being preferred, and comprising the further active compound from groups A to I indicated in each case in the line in question (component 2). According to one embodiment of the invention, component 1 in each line of Table C is in each case one of the compounds of the formula I that are specifically individualized in Tables 1a to 9a. The active compounds in the compositions described are in each case preferably present in synergistically effective amounts.

TABLE C
Active compound composition, comprising an individualized compound I
and a further active compound from groups A) to I).

| Row | Component 1 | Component 2 |
|---|---|---|
| C-1 | a compound of the formula I | azoxystrobin |
| C-2 | a compound of the formula I | dimoxystrobin |
| C-3 | a compound of the formula I | enestroburin |
| C-4 | a compound of the formula I | fluoxastrobin |
| C-5 | a compound of the formula I | kresoxim-methyl |
| C-6 | a compound of the formula I | metominostrobin |
| C-7 | a compound of the formula I | orysastrobin |
| C-8 | a compound of the formula I | picoxystrobin |
| C-9 | a compound of the formula I | pyraclostrobin |
| C-10 | a compound of the formula I | pyribencarb |
| C-11 | a compound of the formula I | trifloxystrobin |
| C-12 | a compound of the formula I | 2-(2-(6-(3-chloro-2-methylphenoxy)- |
| | | 5-fluoropyrimidin-4-yloxy)phenyl)- |
| | | 2-methoxyimino-N-methylacetamide |
| C-13 | a compound of the formula I | 2-(ortho-((2,5-dimethylphenyloxy- |
| | | methylene)phenyl)-3-methoxyacrylic acid methyl |
| | | ester |
| C-14 | a compound of the formula I | 3-methoxy-2-(2-(N-(4-methoxyphenyl)- |
| | | cyclopropanecarboximidoylsulfanyl- |
| | | methyl)phenyl)acrylic acid methyl ester |
| C-15 | a compound of the formula I | benalaxyl |
| C-16 | a compound of the formula I | benalaxyl-M |
| C-17 | a compound of the formula I | benodanil |
| C-18 | a compound of the formula I | bixafen |
| C-19 | a compound of the formula I | boscalid |
| C-20 | a compound of the formula I | carboxin |
| C-21 | a compound of the formula I | fenfuram |
| C-22 | a compound of the formula I | fenhexamid |
| C-23 | a compound of the formula I | flutolanil |
| C-24 | a compound of the formula I | furametpyr |
| C-25 | a compound of the formula I | isotianil |
| C-26 | a compound of the formula I | kiralaxyl |
| C-27 | a compound of the formula I | mepronil |
| C-28 | a compound of the formula I | metalaxyl |
| C-29 | a compound of the formula I | ofurace |
| C-30 | a compound of the formula I | oxadixyl |
| C-31 | a compound of the formula I | oxycarboxin |
| C-32 | a compound of the formula I | penthiopyrad |
| C-33 | a compound of the formula I | sedaxane |
| C-34 | a compound of the formula I | thifluzamide |
| C-35 | a compound of the formula I | tecloftalam |
| C-36 | a compound of the formula I | tiadinil |
| C-37 | a compound of the formula I | 2-amino-4-methylthiazole-5-carboxanilide |
| C-38 | a compound of the formula I | 2-chloro-N-(1,1,3-trimethylindan-4-yl)- |
| | | nicotinamide |
| C-39 | a compound of the formula I | N-(3′,4′-dichloro-5-fluorobiphenyl-2-yl)- |
| | | 3-difluoromethyl-1-methyl-1H-pyrazole- |
| | | 4-carboxamide |
| C-40 | a compound of the formula I | 5-fluoro-1,3-dimethyl-1H-pyrazole- |
| | | 4-carboxylic acid [2-(1,3-dimethylbutyl)- |
| | | phenyl]amide |
| C-41 | a compound of the formula I | N-(4′-chloro-3′,5-difluorobiphenyl-2-yl)- |
| | | 3-difluoromethyl-1-methyl-1H-pyrazole- |
| | | 4-carboxamide |
| C-42 | a compound of the formula I | N-(4′-chloro-3′,5-difluorobiphenyl-2-yl)- |
| | | 3-trifluoromethyl-1-methyl-1H-pyrazole- |
| | | 4-carboxamide |
| C-43 | a compound of the formula I | N-(3′,4′-dichloro-4-fluorobiphenyl-2-yl)- |
| | | 3-trifluoromethyl-1-methyl-1H-pyrazole- |
| | | 4-carboxamide |
| C-44 | a compound of the formula I | N-(3′,5-difluoro-4′-methylbiphenyl-2-yl)- |
| | | 3-difluoromethyl-1-methyl-1H-pyrazole- |
| | | 4-carboxamide |
| C-45 | a compound of the formula I | N-(3′,5-difluoro-4′-methylbiphenyl-2-yl)- |
| | | 3-trifluoromethyl-1-methyl-1H-pyrazole- |
| | | 4-carboxamide |
| C-46 | a compound of the formula I | N-(2-bicyclopropyl-2-ylphenyl)-3-difluoro- |
| | | methyl-1-methyl-1H-pyrazole- |
| | | 4-carboxamide |
| C-47 | a compound of the formula I | N-(cis-2-bicyclopropyl-2-ylphenyl)- |
| | | 3-difluoromethyl-1-methyl-1H-pyrazole- |
| | | 4-carboxamide |
| C-48 | a compound of the formula I | N-(trans-2-bicyclopropyl-2-ylphenyl)-3- |
| | | difluoromethyl-1-methyl-1H-pyrazole- |
| | | 4-carboxamide |
| C-49 | a compound of the formula I | N-(4′-bromobiphenyl-2-yl)-4- |
| | | difluoromethyl-2-methylthiazole-5- |
| | | carboxamide |
| C-50 | a compound of the formula I | N-(4′-trifluoromethylbiphenyl-2-yl)-4- |
| | | difluoromethyl-2-methylthiazole-5- |
| | | carboxamide |
| C-51 | a compound of the formula I | N-(4′-chloro-3′-fluorobiphenyl-2-yl)-4- |
| | | difluoromethyl-2-methylthiazole-5- |
| | | carboxamide |
| C-52 | a compound of the formula I | 3,4-dichloro-N-(2-cyanophenyl)isothiazole- |
| | | 5-carboxamide |
| C-53 | a compound of the formula I | N-(2′,4′-difluorobiphenyl-2-yl)-1-methyl-3- |
| | | trifluoromethyl-1H-pyrazole-4-carboxamide |
| C-54 | a compound of the formula I | N-(2′,4′-dichlorobiphenyl-2-yl)-1-methyl-3- |
| | | trifluoromethyl-1H-pyrazole-4-carboxamide |
| C-55 | a compound of the formula I | N-(2′,4′-difluorobiphenyl-2-yl)-3- |
| | | difluoromethyl-1-methyl-1H-pyrazole-4- |
| | | carboxamide |
| C-56 | a compound of the formula I | N-(2′,4′-dichlorobiphenyl-2-yl)-3- |
| | | difluoromethyl-1-methyl-1H-pyrazole-4- |
| | | carboxamide |
| C-57 | a compound of the formula I | N-(2′,5′-difluorobiphenyl-2-yl)-1-methyl-3- |
| | | trifluoromethyl-1H-pyrazole-4-carboxamide |
| C-58 | a compound of the formula I | N-(2′,5′-dichlorobiphenyl-2-yl)-1-methyl-3- |
| | | trifluoromethyl-1H-pyrazole-4-carboxamide |
| C-59 | a compound of the formula I | N-(2′,5′-difluorobiphenyl-2-yl)-3- |
| | | difluoromethyl-1-methyl-1H-pyrazole-4- |
| | | carboxamide |
| C-60 | a compound of the formula I | N-(2′,5′-dichlorobiphenyl-2-yl)-3- |
| | | difluoromethyl-1-methyl-1H-pyrazole-4- |
| | | carboxamide |
| C-61 | a compound of the formula I | N-(3′,5′-difluorobiphenyl-2-yl)-1-methyl-3- |
| | | trifluoromethyl-1H-pyrazole-4-carboxamide |
| C-62 | a compound of the formula I | N-(3′,5′-dichlorobiphenyl-2-yl)-1-methyl-3- |
| | | trifluoromethyl-1H-pyrazole-4-carboxamide |
| C-63 | a compound of the formula I | N-(3′,5′-difluorobiphenyl-2-yl)-3- |
| | | difluoromethyl-1-methyl-1H-pyrazole-4- |
| | | carboxamide |
| C-64 | a compound of the formula I | N-(3′,5′-dichlorobiphenyl-2-yl)-3- |
| | | difluoromethyl-1-methyl-1H-pyrazole-4- |
| | | carboxamide |
| C-65 | a compound of the formula I | N-(3′-fluorobiphenyl-2-yl)-1-methyl-3- |
| | | trifluoromethyl-1H-pyrazole-4-carboxamide |
| C-66 | a compound of the formula I | N-(3′-chlorobiphenyl-2-yl)-1-methyl-3- |
| | | trifluoromethyl-1H-pyrazole-4-carboxamide |
| C-67 | a compound of the formula I | N-(3′-fluorobiphenyl-2-yl)-3-difluoromethyl- |
| | | 1-methyl-1H-pyrazole-4-carboxamide |
| C-68 | a compound of the formula I | N-(3′-chlorobiphenyl-2-yl)-3-difluoromethyl- |
| | | 1-methyl-1H-pyrazole-4-carboxamide |
| C-69 | a compound of the formula I | N-(2′-fluorobiphenyl-2-yl)-1-methyl-3- |
| | | trifluoromethyl-1H-pyrazole-4-carboxamide |
| C-70 | a compound of the formula I | N-(2′-chlorobiphenyl-2-yl)-1-methyl-3- |
| | | trifluoromethyl-1H-pyrazole-4-carboxamide |
| C-71 | a compound of the formula I | N-(2′-fluorobiphenyl-2-yl)-3-difluoromethyl- |
| | | 1-methyl-1H-pyrazole-4-carboxamide |
| C-72 | a compound of the formula I | N-(2′-chlorobiphenyl-2-yl)-3-difluoromethyl- |
| | | 1-methyl-1H-pyrazole-4-carboxamide |
| C-73 | a compound of the formula I | N-(2′-fluoro-4′-chloro-5′-methylbiphenyl-2- |
| | | yl)-1-methyl-3-trifluoromethyl-1H-pyrazole- |
| | | 4-carboxamide |
| C-74 | a compound of the formula I | N-(3′,4′,5′-trifluorobiphenyl-2-yl)-1-methyl- |
| | | 3-trifluoromethyl-1H-pyrazole-4- |
| | | carboxamide |
| C-75 | a compound of the formula I | N-(3′,4′,5′-trifluorobiphenyl-2-yl)-1-methyl- |
| | | 3-difluoromethyl-1H-pyrazole-4- |
| | | carboxamide |
| C-76 | a compound of the formula I | N-(2′,4′,5′-trifluorobiphenyl-2-yl)-1-methyl- |
| | | 3-difluoromethyl-1H-pyrazole-4- |
| | | carboxamide |
| C-77 | a compound of the formula I | N-(3′,4′,5′-trifluorobiphenyl-2-yl)-3- |
| | | chlorofluoromethyl-1-methyl-1H-pyrazole- |
| | | 4-carboxamide |
| C-78 | a compound of the formula I | N-[2-(1,1,2,3,3,3- |
| | | hexafluoropropoxy)phenyl]-1-methyl-3- |
| | | trifluoromethyl-1H-pyrazole-4-carboxamide |
| C-79 | a compound of the formula I | N-[2-(1,1,2,3,3,3-hexafluoropropoxy)- |
| | | phenyl]-3-difluoromethyl-1-methyl-1H- |
| | | pyrazole-4-carboxamide |
| C-80 | a compound of the formula I | N-[2-(2-chloro-1,1,2-trifluoroethoxy)- |
| | | phenyl]-1-methyl-3-trifluoromethyl-1H- |
| | | pyrazole-4-carboxamide |
| C-81 | a compound of the formula I | N-[2-(2-chloro-1,1,2- |
| | | trifluoroethoxy)phenyl]-3-difluoromethyl-1- |
| | | methyl-1H-pyrazole-4-carboxamide |
| C-82 | a compound of the formula I | N-[2-(1,1,2,2-tetrafluoroethoxy)phenyl]-3- |
| | | difluoromethyl-1-methyl-1H-pyrazole-4- |
| | | carboxamide |
| C-83 | a compound of the formula I | N-[2-(1,1,2,2-tetrafluorethoxy)phenyl]-1- |
| | | methyl-3-trifluoromethyl-1H-pyrazole-4- |
| | | carboxamide |
| C-84 | a compound of the formula I | N-(4′-(trifluoromethylthio)biphenyl-2-yl)-3- |
| | | difluoromethyl-1-methyl-1H-pyrazole-4- |
| | | carboxamide |
| C-85 | a compound of the formula I | N-(4′-(trifluoromethylthio)biphenyl-2-yl)-1- |
| | | methyl-3-trifluoromethyl-1H-pyrazole-4- |
| | | carboxamide |
| C-86 | a compound of the formula I | [2-(1,2-dimethylpropyl)phenyl]-5-fluoro- |
| | | 1,3-dimethyl-1H-pyrazole-4-carboxamide |
| C-87 | a compound of the formula I | N-(2-{4-[3-(4-chlorophenyl)prop-2- |
| | | ynyloxy]-3-methoxyphenyl}ethyl)-2- |
| | | methanesulfonylamino-3- |
| | | methylbutyramide |
| C-88 | a compound of the formula I | N-(2-{4-[3-(4-chlorophenyl)prop-2- |
| | | ynyloxy]-3-methoxyphenyl}ethyl)-2- |
| | | ethanesulfonylamino-3-methylbutyramide |
| C-89 | a compound of the formula I | dimethomorph |
| C-90 | a compound of the formula I | flumorph |
| C-91 | a compound of the formula I | flumetover |
| C-92 | a compound of the formula I | fluopicolide (picobenzamid) |
| C-93 | a compound of the formula I | fluopyram |
| C-94 | a compound of the formula I | zoxamide |
| C-95 | a compound of the formula I | N-(3-ethyl-3,5,5-trimethylcyclohexyl)- |
| | | 3-formylamino-2-hydroxybenzamide |
| C-96 | a compound of the formula I | carpropamid |
| C-97 | a compound of the formula I | diclocymet |
| C-98 | a compound of the formula I | mandipropamid |
| C-99 | a compound of the formula I | oxytetracyclin |
| C-100 | a compound of the formula I | silthiofam |
| C-101 | a compound of the formula I | N-(6-methoxypyridin-3-yl)cyclopropane- |
| | | carboxamide |
| C-102 | a compound of the formula I | azaconazole |
| C-103 | a compound of the formula I | bitertanol |
| C-104 | a compound of the formula I | bromuconazole |
| C-105 | a compound of the formula I | cyproconazole |
| C-106 | a compound of the formula I | difenoconazole |
| C-107 | a compound of the formula I | diniconazole |
| C-108 | a compound of the formula I | diniconazole-M |
| C-109 | a compound of the formula I | enilconazole |
| C-110 | a compound of the formula I | epoxiconazole |
| C-111 | a compound of the formula I | fenbuconazole |
| C-112 | a compound of the formula I | flusilazole |
| C-113 | a compound of the formula I | fluquinconazole |
| C-114 | a compound of the formula I | flutriafol |
| C-115 | a compound of the formula I | hexaconazole |
| C-116 | a compound of the formula I | imibenconazole |
| C-117 | a compound of the formula I | ipconazole |
| C-118 | a compound of the formula I | metconazole |
| C-119 | a compound of the formula I | myclobutanil |
| C-120 | a compound of the formula I | oxpoconazole |
| C-121 | a compound of the formula I | paclobutrazole |
| C-122 | a compound of the formula I | penconazole |
| C-123 | a compound of the formula I | propiconazole |
| C-124 | a compound of the formula I | prothioconazole |
| C-125 | a compound of the formula I | simeconazole |
| C-126 | a compound of the formula I | tebuconazole |
| C-127 | a compound of the formula I | tetraconazole |
| C-128 | a compound of the formula I | triadimenol |
| C-129 | a compound of the formula I | triadimefon |
| C-130 | a compound of the formula I | triticonazole |
| C-131 | a compound of the formula I | uniconazole |
| C-132 | a compound of the formula I | 1-(4-chlorophenyl)-2-([1,2,4]triazol-1-yl)- |
| | | cycloheptanol |
| C-133 | a compound of the formula I | cyazofamid |
| C-134 | a compound of the formula I | imazalil |
| C-135 | a compound of the formula I | imazalil-sulfate |
| C-136 | a compound of the formula I | pefurazoate |
| C-137 | a compound of the formula I | prochloraz |
| C-138 | a compound of the formula I | triflumizole |
| C-139 | a compound of the formula I | benomyl |
| C-140 | a compound of the formula I | carbendazim |
| C-141 | a compound of the formula I | fuberidazole |
| C-142 | a compound of the formula I | thiabendazole |
| C-143 | a compound of the formula I | ethaboxam |
| C-144 | a compound of the formula I | etridiazole |
| C-145 | a compound of the formula I | hymexazole |
| C-146 | a compound of the formula I | fluazinam |
| C-147 | a compound of the formula I | pyrifenox |
| C-148 | a compound of the formula I | 1-(4-chlorophenyl)-1-(propyn-2-yloxy)- |
| | | 3-(4-(3,4-dimethoxyphenyl)isoxazol-5-yl)- |
| | | propan-2-one |
| C-149 | a compound of the formula I | 3-[5-(4-chlorophenyl)-2,3-dimethyl- |
| | | isoxazolidin-3-yl]pyridine |
| C-150 | a compound of the formula I | 2,3,5,6-tetrachloro-4-methanesulfonyl- |
| | | pyridine |
| C-151 | a compound of the formula I | 3,4,5-trichloropyridine-2,6-dicarbonitrile |
| C-152 | a compound of the formula I | N-(1-(5-bromo-3-chloropyridin-2-yl)ethyl)- |
| | | 2,4-dichloronicotinamide |
| C-153 | a compound of the formula I | N-((5-bromo-3-chloropyridin-2-yl)methyl)- |
| | | 2,4-dichloronicotinamide |
| C-154 | a compound of the formula I | bupirimate |
| C-155 | a compound of the formula I | cyprodinil |
| C-156 | a compound of the formula I | diflumetorim |
| C-157 | a compound of the formula I | ferimzone |
| C-158 | a compound of the formula I | fenarimol |
| C-159 | a compound of the formula I | mepanipyrim |
| C-160 | a compound of the formula I | nitrapyrin |
| C-161 | a compound of the formula I | nuarimol |
| C-162 | a compound of the formula I | pyrimethanil |
| C-163 | a compound of the formula I | fludioxonil |
| C-164 | a compound of the formula I | fenpiclonil |
| C-165 | a compound of the formula I | aldimorph |
| C-166 | a compound of the formula I | dodemorph |
| C-167 | a compound of the formula I | dodemorph acetate |
| C-168 | a compound of the formula I | fenpropimorph |
| C-169 | a compound of the formula I | tridemorph |
| C-170 | a compound of the formula I | fluoroimid |
| C-171 | a compound of the formula I | iprodione |
| C-172 | a compound of the formula I | procymidone |
| C-173 | a compound of the formula I | vinclozolin |
| C-174 | a compound of the formula I | acibenzolar-S-methyl |
| C-175 | a compound of the formula I | amisulbrom |
| C-176 | a compound of the formula I | anilazin |
| C-177 | a compound of the formula I | blasticidin-S |
| C-178 | a compound of the formula I | captan |
| C-179 | a compound of the formula I | captafol |
| C-180 | a compound of the formula I | quinomethionate |
| C-181 | a compound of the formula I | dazomet |
| C-182 | a compound of the formula I | debacarb |
| C-183 | a compound of the formula I | diclomezine |
| C-184 | a compound of the formula I | difenzoquat |
| C-185 | a compound of the formula I | difenzoquat methylsulfate |
| C-186 | a compound of the formula I | famoxadone |
| C-187 | a compound of the formula I | fenamidone |
| C-188 | a compound of the formula I | fenoxanil |
| C-189 | a compound of the formula I | fenpropidin |
| C-190 | a compound of the formula I | folpet |
| C-191 | a compound of the formula I | octhilinone |
| C-192 | a compound of the formula I | oxolinic acid |
| C-193 | a compound of the formula I | piperalin |
| C-194 | a compound of the formula I | probenazole |
| C-195 | a compound of the formula I | proquinazid |
| C-196 | a compound of the formula I | pyroquilon |
| C-197 | a compound of the formula I | quinoxyfen |
| C-198 | a compound of the formula I | triazoxid |
| C-199 | a compound of the formula I | tricyclazole |
| C-200 | a compound of the formula I | triforine |
| C-201 | a compound of the formula I | 5-chloro-7-(4-methylpiperidin-1-yl)- |
| | | 6-(2,4,6-trifluorophenyl)-[1,2,4]triazolo- |
| | | [1,5-a]pyrimidine |
| C-202 | a compound of the formula I | 6-(4-tert-butylphenyl)-5-methyl- |
| | | [1,2,4]triazolo[1,5-a]pyrimidin-7-ylamine |
| C-203 | a compound of the formula I | 5-methyl-6-(3,5,5-trimethylhexyl)- |
| | | [1,2,4]triazolo[1,5-a]pyrimidin-7-ylamine |
| C-204 | a compound of the formula I | 5-methyl-6-octyl-[1,2,4]triazolo[1,5-a]- |
| | | pyrimidin-7-ylamine |
| C-205 | a compound of the formula I | 6-methyl-5-octyl-[1,2,4]triazolo[1,5-a]- |
| | | pyrimidin-7-ylamine |
| C-206 | a compound of the formula I | 6-ethyl-5-octyl-[1,2,4]triazolo[1,5-a]- |
| | | pyrimidin-7-ylamine |
| C-207 | a compound of the formula I | 5-ethyl-6-octyl-[1,2,4]triazolo[1,5- |
| | | a]pyrimidin-7-ylamine |
| C-208 | a compound of the formula I | 5-ethyl-6-(3,5,5-trimethylhexyl)- |
| | | [1,2,4]triazolo[1,5-a]pyrimidin-7-ylamine |
| C-209 | a compound of the formula I | 6-octyl-5-propyl-[1,2,4]triazolo[1,5-a]- |
| | | pyrimidin-7-ylamine |
| C-210 | a compound of the formula I | 5-methoxymethyl-6-octyl- |
| | | [1,2,4]triazolo[1,5-a]pyrimidin-7-ylamine |
| C-211 | a compound of the formula I | 6-octyl-5-trifluoromethyl[1,2,4]triazolo[1,5- |
| | | a]pyrimidin-7-ylamine |
| C-212 | a compound of the formula I | 5-trifluoromethyl-6-(3,5,5-trimethylhexyl)- |
| | | [1,2,4]triazolo[1,5-a]pyrimidin-7-ylamine |
| C-213 | a compound of the formula I | 2-butoxy-6-iodo-3-propylchromen-4-one |
| C-214 | a compound of the formula I | ferbam |
| C-215 | a compound of the formula I | mancozeb |
| C-216 | a compound of the formula I | maneb |
| C-217 | a compound of the formula I | metiram |
| C-218 | a compound of the formula I | metam |
| C-219 | a compound of the formula I | methasulphocarb |
| C-220 | a compound of the formula I | propineb |
| C-221 | a compound of the formula I | thiram |
| C-222 | a compound of the formula I | zineb |
| C-223 | a compound of the formula I | ziram |
| C-224 | a compound of the formula I | diethofencarb |
| C-225 | a compound of the formula I | benthiavalicarb |
| C-226 | a compound of the formula I | iprovalicarb |
| C-227 | a compound of the formula I | propamocarb |
| C-228 | a compound of the formula I | propamocarb hydrochloride |
| C-229 | a compound of the formula I | 3-(4-chlorophenyl)-3-(2-isopropoxy- |
| | | carbonylamino-3-methylbutyrylamino)- |
| | | propionic acid methyl ester |
| C-230 | a compound of the formula I | valiphenal |
| C-231 | a compound of the formula I | 4-fluorophenyl N-(1-(1-(4-cyanophenyl)- |
| | | ethanesulfonyl)but-2-yl)carbamate |
| C-232 | a compound of the formula I | dodine |
| C-233 | a compound of the formula I | dodine free base |
| C-234 | a compound of the formula I | iminoctadine |
| C-235 | a compound of the formula I | iminoctadine triacetate |
| C-236 | a compound of the formula I | iminoctadine tris(albesilate) |
| C-237 | a compound of the formula I | guazatine |
| C-238 | a compound of the formula I | guazatine acetate |
| C-239 | a compound of the formula I | kasugamycin |
| C-240 | a compound of the formula I | kasugamycin hydrochloride hydrate |
| C-241 | a compound of the formula I | polyoxine |
| C-242 | a compound of the formula I | streptomycin |
| C-243 | a compound of the formula I | validamycin A |
| C-244 | a compound of the formula I | binapacryl |
| C-245 | a compound of the formula I | dicloran |
| C-246 | a compound of the formula I | dinobuton |
| C-247 | a compound of the formula I | dinocap |
| C-248 | a compound of the formula I | nitrothal-isopropyl |
| C-249 | a compound of the formula I | tecnazen |
| C-250 | a compound of the formula I | fentin acetate |
| C-251 | a compound of the formula I | fentin chloride |
| C-252 | a compound of the formula I | fentin hydroxide |
| C-253 | a compound of the formula I | isoprothiolane |
| C-254 | a compound of the formula I | dithianon |
| C-255 | a compound of the formula I | edifenphos |
| C-256 | a compound of the formula I | fosetyl |
| C-257 | a compound of the formula I | fosetyl aluminum |
| C-258 | a compound of the formula I | iprobenfos |
| C-259 | a compound of the formula I | pyrazophos |
| C-260 | a compound of the formula I | tolclofos-methyl |
| C-261 | a compound of the formula I | chlorothalonil |
| C-262 | a compound of the formula I | dichlofluanid |
| C-263 | a compound of the formula I | dichlorophen |
| C-264 | a compound of the formula I | flusulfamide |
| C-265 | a compound of the formula I | hexachlorobenzene |
| C-266 | a compound of the formula I | pencycuron |
| C-267 | a compound of the formula I | pentachlorophenol and salts thereof |
| C-268 | a compound of the formula I | phthalide |
| C-269 | a compound of the formula I | quintozene |
| C-270 | a compound of the formula I | thiophanate methyl |
| C-271 | a compound of the formula I | tolylfluanid |
| C-272 | a compound of the formula I | N-(4-chloro-2-nitrophenyl)-N-ethyl- |
| | | 4-methylbenzenesulfonamide |
| C-273 | a compound of the formula I | phosphorous acid and its salts |
| C-274 | a compound of the formula I | sulfur |
| C-275 | a compound of the formula I | Bordeaux mixture |
| C-276 | a compound of the formula I | copper acetate |
| C-277 | a compound of the formula I | copper hydroxide |
| C-278 | a compound of the formula I | copper oxychloride |
| C-279 | a compound of the formula I | basic copper sulfate |
| C-280 | a compound of the formula I | biphenyl |
| C-281 | a compound of the formula I | bronopol |
| C-282 | a compound of the formula I | cyflufenamid |
| C-283 | a compound of the formula I | cymoxanil |
| C-284 | a compound of the formula I | diphenylamine |
| C-285 | a compound of the formula I | metrafenon |
| C-286 | a compound of the formula I | mildiomycin |
| C-287 | a compound of the formula I | oxine-copper |
| C-288 | a compound of the formula I | prohexadione-calcium |
| C-289 | a compound of the formula I | spiroxamine |
| C-290 | a compound of the formula I | tolylfluanid |
| C-291 | a compound of the formula I | N-(cyclopropylmethoxyimino-(6-difluoro- |
| | | methoxy-2,3-difluorophenyl)methyl)- |
| | | 2-phenylacetamide |
| C-292 | a compound of the formula I | N′-(4-(4-chloro-3-trifluoromethylphenoxy)- |
| | | 2,5-dimethylphenyl)-N-ethyl-N-methyl- |
| | | formamidine |
| C-293 | a compound of the formula I | N′-(4-(4-fluoro-3-trifluoromethylphenoxy)- |
| | | 2,5-dimethylphenyl)-N-ethyl-N-methyl- |
| | | formamidine |
| C-294 | a compound of the formula I | N′-(2-methyl-5-trifluoromethyl-4-(3-tri- |
| | | methylsilanylpropoxy)phenyl)-N-ethyl- |
| | | N-methylformamidine |
| C-295 | a compound of the formula I | N′-(5-difluoromethyl-2-methyl-4-(3-tri- |
| | | methylsilanylpropoxy)phenyl)-N-ethyl- |
| | | N-methylformamidine |
| C-296 | a compound of the formula I | 2-(2-(3-(2,6-dichlorophenyl)-1-methyl- |
| | | allylideneaminooxymethyl)phenyl)-2- |
| | | methoxyimino-N-methylacetamide |
| C-297 | a compound of the formula I | isopyrazam |
| C-298 | a compound of the formula I | metalaxyl-M (mefenoxam) |
| C-299 | a compound of the formula I | N-(3′,4′-dichloro-5-fluorobiphenyl-2-yl)-3- |
| | | trifluoromethyl-1-methyl-1H-pyrazole-4- |
| | | carbonic acid amide |
| C-300 | a compound of the formula I | N-(2-(1,3,3-trimethylbutyl)phenyl)-1,3- |
| | | dimethyl-5-fluoro-1H-pyrazole-4- |
| | | carboxamide |
| C-301 | a compound of the formula I | N-[1,2,3,4-tetrahydro-9-(1-methylethyl)- |
| | | 1,4-methanonaphthalen-5-yl]-3- |
| | | (difluoromethyl)-1-methyl-1H-pyrazole-4- |
| | | carboxamide |
| C-302 | a compound of the formula I | N′-(4-(4-chloro-3-trifluoromethylphenoxy)- |
| | | 2,5-dimethylphenyl)-N-ethyl-N-methyl- |
| | | formamidine |
| C-303 | a compound of the formula I | N′-(4-(4-fluoro-3-trifluoromethylphenoxy)- |
| | | 2,5-dimethylphenyl)-N-ethyl-N-methyl- |
| | | formamidine |
| C-304 | a compound of the formula I | N′-(2-methyl-5-trifluoromethyl-4-(3- |
| | | trimethylsilanylpropoxy)phenyl)-N-ethyl-N- |
| | | methylformamidine |
| C-305 | a compound of the formula I | N′-(5-difluoromethyl-2-methyl-4-(3- |
| | | trimethylsilanylpropoxy)phenyl)-N-ethyl-N- |
| | | methylformamidine |
| C-306 | a compound of the formula I | 2-(4-chlorophenyl)-N-[4-(3,4-dimethoxy- |
| | | phenyl)isoxazol-5-yl]-2-prop-2-ynyloxy- |
| | | acetamide |
| C-307 | a compound of the formula I | 3-[5-(4-methylphenyl)-2,3-dimethyl- |
| | | isoxazolidin-3-yl]pyridine, |
| C-308 | a compound of the formula I | flutianil |
| C-309 | a compound of the formula I | S-allyl 5-amino-2-isopropyl-3-oxo-4- |
| | | orthotolyl-2,3-dihydropyrazole-1- |
| | | thiocarbonate |
| C-310 | a compound of the formula I | 5-chloro-1-(4,6-dimethoxypyrimidin-2-yl)-2- |
| | | methyl-1H-benzoimidazole |
| C-311 | a compound of the formula I | 6-(3,4-dichlorophenyl)-5-methyl-[1,2,4]tri- |
| | | azolo[1,5-a]pyrimidin-7-ylamine |
| C-312 | a compound of the formula I | 2-{1-[2-(5-methyl-3-trifluoromethylpyrazol- |
| | | 1-yl)acetyl]piperidin-4-yl}thiazole-4- |
| | | carboxylic acid methyl-(1,2,3,4-tetra- |
| | | hydronaphthalen-1-yl)amide |
| C-313 | a compound of the formula I | 2-{1-[2-(5-methyl-3-trifluoromethylpyrazol- |
| | | 1-yl)acetyl]piperidin-4-yl}thiazole-4- |
| | | carboxylic acid methyl-(R)-1,2,3,4- |
| | | tetrahydronaphthalen-1-ylamide |
| C-314 | a compound of the formula I | 6-tert-butyl-8-fluoro-2,3-dimethylquinolin-4- |
| | | yl acetate |
| C-315 | a compound of the formula I | 6-tert-butyl-8-fluoro-2,3-dimethylquinolin-4- |
| | | yl methoxyacetate |
| C-316 | a compound of the formula I | carbaryl |
| C-317 | a compound of the formula I | carbofuran |
| C-318 | a compound of the formula I | carbosulfan |
| C-319 | a compound of the formula I | methomylthiodicarb |
| C-320 | a compound of the formula I | bifenthrin |
| C-321 | a compound of the formula I | cyfluthrin |
| C-322 | a compound of the formula I | cypermethrin |
| C-323 | a compound of the formula I | alpha-cypermethrin |
| C-324 | a compound of the formula I | zeta-cypermethrin |
| C-325 | a compound of the formula I | deltamethrin |
| C-326 | a compound of the formula I | esfenvalerate |
| C-327 | a compound of the formula I | lambda-cyhalothrin |
| C-328 | a compound of the formula I | permethrin |
| C-329 | a compound of the formula I | tefluthrin |
| C-330 | a compound of the formula I | diflubenzuron |
| C-331 | a compound of the formula I | flufenoxuron |
| C-332 | a compound of the formula I | lufenuron |
| C-333 | a compound of the formula I | teflubenzuron |
| C-334 | a compound of the formula I | spirotetramate |
| C-335 | a compound of the formula I | clothianidin |
| C-336 | a compound of the formula I | dinotefuran |
| C-337 | a compound of the formula I | imidacloprid |
| C-338 | a compound of the formula I | thiamethoxam |
| C-339 | a compound of the formula I | acetamiprid |
| C-340 | a compound of the formula I | thiacloprid |
| C-341 | a compound of the formula I | endosulfan |
| C-342 | a compound of the formula I | fipronil |
| C-343 | a compound of the formula I | abamectin |
| C-344 | a compound of the formula I | emamectin |
| C-345 | a compound of the formula I | spinosad |
| C-346 | a compound of the formula I | spinetoram |
| C-347 | a compound of the formula I | hydramethylnon |
| C-348 | a compound of the formula I | chlorfenapyr |
| C-349 | a compound of the formula I | fenbutatin oxide |
| C-350 | a compound of the formula I | indoxacarb |
| C-351 | a compound of the formula I | metaflumizone |
| C-352 | a compound of the formula I | flonicamid |
| C-353 | a compound of the formula I | lubendiamide |
| C-354 | a compound of the formula I | chlorantraniliprol |
| C-355 | a compound of the formula I | cyazypyr (HGW86) |
| C-356 | a compound of the formula I | cyflumetofen |
| C-357 | a compound of the formula I | acetochlor |
| C-358 | a compound of the formula I | dimethenamid |
| C-359 | a compound of the formula I | metolachlor |
| C-360 | a compound of the formula I | metazachlor |
| C-361 | a compound of the formula I | glyphosate |
| C-362 | a compound of the formula I | glufosinate |
| C-363 | a compound of the formula I | sulfosate |
| C-364 | a compound of the formula I | clodinafop |
| C-365 | a compound of the formula I | fenoxaprop |
| C-366 | a compound of the formula I | fluazifop |
| C-367 | a compound of the formula I | haloxyfop |
| C-368 | a compound of the formula I | paraquat |
| C-369 | a compound of the formula I | phenmedipham |
| C-370 | a compound of the formula I | clethodim |
| C-371 | a compound of the formula I | cycloxydim |
| C-372 | a compound of the formula I | profoxydim |
| C-373 | a compound of the formula I | sethoxydim |
| C-374 | a compound of the formula I | tepraloxydim |
| C-375 | a compound of the formula I | pendimethalin |
| C-376 | a compound of the formula I | prodiamine |
| C-377 | a compound of the formula I | trifluralin |
| C-378 | a compound of the formula I | acifluorfen |
| C-379 | a compound of the formula I | bromoxynil |
| C-380 | a compound of the formula I | imazamethabenz |
| C-381 | a compound of the formula I | imazamox |
| C-382 | a compound of the formula I | imazapic |
| C-383 | a compound of the formula I | imazapyr |
| C-384 | a compound of the formula I | imazaquin |
| C-385 | a compound of the formula I | imazethapyr |
| C-386 | a compound of the formula I | 2,4-dichlorophenoxyacetic acid (2,4-D) |
| C-387 | a compound of the formula I | chloridazon |
| C-388 | a compound of the formula I | clopyralid |
| C-389 | a compound of the formula I | fluroxypyr |
| C-390 | a compound of the formula I | picloram |
| C-391 | a compound of the formula I | picolinafen |
| C-392 | a compound of the formula I | bensulfuron |
| C-393 | a compound of the formula I | chlorimuron-ethyl |
| C-394 | a compound of the formula I | cyclosulfamuron |
| C-395 | a compound of the formula I | iodosulfuron |
| C-396 | a compound of the formula I | mesosulfuron |
| C-397 | a compound of the formula I | metsulfuron-methyl |
| C-398 | a compound of the formula I | nicosulfuron |
| C-399 | a compound of the formula I | rimsulfuron |
| C-400 | a compound of the formula I | triflusulfuron |
| C-401 | a compound of the formula I | atrazine |
| C-402 | a compound of the formula I | hexazinon |
| C-403 | a compound of the formula I | diuron |
| C-404 | a compound of the formula I | florasulam |
| C-405 | a compound of the formula I | pyroxasulfon |
| C-406 | a compound of the formula I | bentazone |
| C-407 | a compound of the formula I | cinidon-ethyl |
| C-408 | a compound of the formula I | cinmethylin |
| C-409 | a compound of the formula I | dicamba |
| C-410 | a compound of the formula I | diflufenzopyr |
| C-411 | a compound of the formula I | quinclorac |
| C-412 | a compound of the formula I | quinmerac |
| C-413 | a compound of the formula I | mesotrione |
| C-414 | a compound of the formula I | saflufenacil |
| C-415 | a compound of the formula I | topramezone |
| C-416 | a compound of the formula I | pyrimorph |

The active compounds specified above as component 2, their preparation, and their effect against fungal pathogens are known (cf.: http://www.hclrss.demon.co.uk/index.html; http://alanwood.net/pesticides/); they are available commercially. The compounds with IUPAC nomenclature, their preparation, and their fungicidal activity are likewise known (cf. Can. J. Plant Sci. 48 (6), 587-94, 1968; EP-A 141 317; EP-A 152 031; EP-A 226 917; EP-A 243 970; EP-A 256 503; EP-A 428 941; EP-A 532 022; EP-A 1 028 125; EP-A 1 035 122; EP-A 1 201 648; EP-A 1 122 244; JP 2002316902; DE 19650197; DE 10021412; DE 102005009458; U.S. Pat. No. 3,296,272; U.S. Pat. No. 3,325,503; WO 98/46608; WO 99/14187; WO 99/24413; WO 99/27783; WO 00/29404; WO 00/46148; WO 00/65913; WO 01/54501; WO 01/56358; WO 02/22583; WO 02/40431; WO 03/10149; WO 03/11853; WO 03/14103; WO 03/16286; WO 03/53145; WO 03/61388; WO 03/66609; WO 03/74491; WO 04/49804; WO 05/120234; WO 05/123689; WO 05/123690; WO 05/63721; WO 05/87772; WO 05/87773; WO 06/15866; WO 06/87325; WO 06/87343; WO 07/82098; WO 07/90624).

The preparation of the compositions for mixtures of active compounds is carried out in a known manner in the form of compositions comprising a solvent or solid carrier in addition to the active compounds, e.g. in the manner as indicated for compositions of the compounds I.

With respect to the customary constituents of such compositions, reference is made to the details on the compositions comprising the compounds I.

The compositions for mixtures of active compounds are suitable as fungicides for the control of harmful fungi. They are distinguished by an outstanding activity against a broad spectrum of phytopathogenic fungi including soil-borne pathogens that are derived, in particular, from the classes of the Plasmodiophoromycetes, Peronosporomycetes (syn. Oomycetes), Chytridiomycetes, Zygomycetes, Ascomycetes, Basidiomycetes and Deuteromycetes (syn. Fungi imperfecti). In addition, reference is made to the details on the activity of the compounds I and the compositions comprising the compounds I.

The present invention also provides the use of compounds I and their pharmaceutically acceptable salts for treating diseases, in particular to the use of the compounds I as antimycotic. Thus, one embodiment of the invention relates to a medicament comprising at least one compound of the formula I and/or a pharmaceutically acceptable salt thereof. A further embodiment relates to the use of a compound I and/or a pharmaceutically effective salt thereof for preparing an antimycotic.

The present invention furthermore provides the use of compounds II and their pharmaceutically acceptable salts for treating diseases, in particular to the use of the compounds II as antimycotic. Thus, one embodiment of the invention relates to a medicament comprising at least one compound of the formula II and/or a pharmaceutically acceptable salt thereof. A further embodiment relates to the use of a compound II and/or a pharmaceutically effective salt thereof for preparing an antimycotic.

Yet a further subject of the present invention is the use of compounds I and their pharmaceutically acceptable salts for the treatment of tumors in mammals, such as, for example, in humans. Thus, one embodiment of the invention relates to the use of a compound I and/or of a pharmaceutically active salt thereof for the production of a composition which inhibits the growth of tumors and cancer in mammals. “Cancer” is in particular intended to mean a malignant tumor, e.g. breast cancer, prostate cancer, lung cancer, CNS cancer, melanocarcinoma, ovarian carcinoma or kidney cancer, in particular in humans.

Yet a further subject of the present invention is the use of compounds I and their pharmaceutically acceptable salts for the treatment of virus infections, in particular virus infections which lead to diseases in warm-blooded animals. Thus, one embodiment of the invention relates to the use of a compound I and/or of a pharmaceutically active salt thereof for the production of a composition for the treatment of virus infections. The diseases to be treated include retrovirus diseases such as, for example: HIV and HTLV, influenza virus, rhinovirus diseases, herpes and the like.

### SYNTHESIS EXAMPLES

The procedures specified in the following synthesis examples were used with corresponding modification of the starting compounds for the obtainment of further compounds of the formula I or of the precursors thereof.

Melting points were obtained on a MeI-Temp II apparatus and are uncorrected. 1H-NMR spectra were measured on a Bruker AC 300 spectrometer at 300 MHz and are based on tetramethylsilane as an internal standard (obtained from Aldrich or Cambridge Isotope Laboratories).

ESI mass spectra were measured on a Shimadzu LCMS-2010 EV mass spectrometer.

APCI mass spectra were measured on a Shimadzu LCMS-2010 EV mass spectrometer.

HPLC analyses were carried out with the aid of an Alltech Alltima C₁₈ Rocket column, with PDA detection at 254 nm, on a Shimadzu Prominence HPLC system, if not stated otherwise. At a flow rate of 2.5 ml per minute, the following time program was used:

| | Percent by volume of A | Percent by volume of B |
|---|---|---|
| Time | (H₂O with | (CH₃CN with |
| [min] | 0.05% trifluoroacetic acid TFA) | 0.05% trifluoroacetic acid TFA) |
| 1.00 | 90 | 10 |
| 4.50 | 0 | 100 |
| 10.00 | 0 | 100 |
| 11.50 | 90 | 10 |

### EXAMPLE 1

### Synthesis of 1-[rel-(2S,3R)-3-(2-chlorophenyl)-2-(3-fluorophenyl)oxiran-2-ylmethyl]-1H-1,2,4-triazole-5(4H)-thione

Lithium diisopropylamide (LDA, 10.0 ml, 19.9 mmol, 2.0 M in THF) was added dropwise at −78° C. to a solution of 1-[rel-(2S,3R)-3-(2-chlorophenyl)-2-(3-fluorophenyl)oxiran-2-ylmethyl]-1H-1,2,4-triazole (5.0 g, 15.3 mmol) in dry tetrahydrofuran (THF, 80 ml). After 30 minutes, sulfur (980 mg, 30.6 mmol) was added. The reaction mixture was stirred for 18 hours, it being allowed to warm from −78° C. to 18° C. The reaction mixture was treated with saturated ammonium chloride solution (30 ml) and then extracted with ethyl acetate (50 ml). The organic phase was washed with saturated sodium chloride solution (three times 40 ml each), dried with sodium sulfate and freed from the solvent. The residue was purified by column chromatography (silica gel, 3:2 hexane/ethyl acetate), recrystallized with hexane/methylene chloride and purified again by column chromatography (silica gel, 3:2 hexane/ethyl acetate). The target compound (2.1 g, 38%) was obtained as a white solid.

1H NMR (300 MHz, CDCl3) δ 7.90 (s, 1H), 7.63-7.19 (m, 7H), 7.04-6.92 (m, 1H), 5.05 (d, 1H), 4.11 (s, 1H), 3.73 (d, 1H).

### EXAMPLE 2

### Synthesis of 1-[rel-(2S,3R)-3-(2-fluorophenyl)-2-(3-fluorophenyl)oxiran-2-ylmethyl]-1H-1,2,4-triazole-5(4H)-thione

n-Butyllithium (2.4 ml, 3.8 mmol, 1.6 M in hexane) was added dropwise at −78° C. to a solution of 1-[rel-(2S,3R)-3-(2-fluorophenyl)-2-(3-fluorophenyl)oxiran-2-ylmethyl]-1H-1,2,4-triazole (1.0 g, 3.2 mmol) in dry tetrahydrofuran (THF, 20 ml). After 20 minutes, sulfur (200 mg, 6.4 mmol) was added. The reaction mixture was stirred at −78° C. for 5 hours. The reaction mixture was treated with methanol (10 ml) and saturated ammonium chloride solution (30 ml) and then extracted with ethyl acetate (50 ml). The organic phase was washed with saturated sodium chloride solution (three times 40 ml each), dried with sodium sulfate and freed from the solvent. The residue was purified by column chromatography (silica gel, 4:1 methylene chloride/ethyl acetate). The target compound (155 mg, 14%) was obtained as a white solid (melting point 125-130° C.).

### EXAMPLE 3

### Synthesis of 1-[rel-(2S,3R)-2-(3-fluorophenyl)-3-o-tolyloxiran-2-ylmethyl]-1H-1,2,4-triazole-5(4H)-thione

Lithium diisopropylamide (LDA, 0.86 ml, 1.72 mmol, 2.0 M in THF) was added dropwise at −78° C. to a solution of 1-[rel-(2S,3R)-2-(3-fluorophenyl)-3-o-tolyloxiran-2-ylmethyl]-1H-1,2,4-triazole (408 mg, 1.32 mmol) in dry tetrahydrofuran (THF, 10 ml). After 20 minutes, sulfur (82 mg, 2.6 mmol) was added. Beginning at −70° C., the reaction mixture was stirred for 4 hours and slowly brought to room temperature overnight. The reaction mixture was treated with saturated ammonium chloride solution (30 ml) and then extracted with ethyl acetate (50 ml). The organic phase was washed with saturated sodium chloride solution (three times 40 ml each), dried with sodium sulfate and freed from the solvent. The residue was purified by recrystallizing from hexane/methylene chloride. The target compound (110 mg, 25%) was obtained as a yellow solid.

1H NMR (300 MHz, CDCl3) δ 7.68 (s, 1H), 7.49 (m, 1H), 7.33-7.15 (m, 6H), 7.00-6.89 (m, 1H), 5.10 (d, 1H), 4.02 (s, 1H), 3.74 (d, 1H), 2.39 (s, 3H).

### EXAMPLE 4

### Synthesis of 1-[rel-(2S,3R)-3-(2-fluorophenyl)-2-(3-fluorophenyl)oxiran-2-ylmethyl]-5-(methylthio)-1H-1,2,4-triazole

n-Butyllithium (0.72 ml, 1.16 mmol, 1.6 M in hexane) was added dropwise at −78° C. to a solution of 1-[rel-(2S,3R)-3-(2-fluorophenyl)-2-(3-fluorophenyl)oxiran-2-ylmethyl]-1H-1,2,4-triazole (300 mg, 0.96 mmol) in dry tetrahydrofuran (THF, 10 ml). After 20 minutes, dimethyl disulfide (86 μl, 0.96 mmol) was added, and the solution was stirred at −78° C. for 30 minutes. The reaction mixture was treated with methanol (10 ml) and saturated ammonium chloride solution (15 ml) and then extracted with ethyl acetate (20 ml). The organic phase was washed with saturated sodium chloride solution (three times 20 ml each), dried with sodium sulfate and freed from the solvent. The residue was purified by column chromatography (silica gel, 13:2 methylene chloride/ethyl acetate). The target compound (140 mg, 41%) was obtained as a white solid (melting point 89-92° C.).

### EXAMPLE 5

### Synthesis of 1-rel-[(2S,3R)-2-(3-fluorophenyl)-3-o-tolyloxiran-2-ylmethyl]-5-(methylthio)-1H-1,2,4-triazole

Lithium diisopropylamide (LDA, 0.40 ml, 0.80 mmol, 2.0 M in THF) was added dropwise at −78° C. to a solution of 1-[rel-(2S,3R)-2-(3-fluorophenyl)-3-o-tolyloxiran-2-ylmethyl]-1H-1,2,4-triazole (192 mg, 0.61 mmol) in dry tetrahydrofuran (THF, 8 ml). After 15 minutes, dimethyl disulfide (83 μl, 0.92 mmol) was added, and the solution was stirred at −78° C. for 4 hours. The reaction mixture was treated with saturated ammonium chloride solution (15 ml) and then extracted with ethyl acetate (20 ml). The organic phase was washed with saturated sodium chloride solution (three times 20 ml each), dried with sodium sulfate and freed from the solvent. The residue was purified by column chromatography (silica gel, 3:1 to 1:1 hexane/ethyl acetate). The target compound (120 mg, 55%) was obtained as a yellow solid.

1H NMR (300 MHz, CDCl3) δ 0.72 (s, 1H), 7.60-7.50 (m, 1H), 7.31-6.91 (m, 7H), 4.51 (d, 1H), 4.08 (s, 1H), 3.94 (d, 1H), 2.48 (s, 3H), 2.41 (s, 3H).

### EXAMPLE 6

### Synthesis of 1-[rel-(2S,3R)-3-(2-chlorophenyl)-2-(3-fluorophenyl)oxiran-2-ylmethyl]-5-(methylthio)-1H-1,2,4-triazole

150 mg of 1-[rel-(2S,3R)-3-(2-chlorophenyl)-2-(3-fluorophenyl)oxiran-2-ylmethyl]-1H-1,2,4-triazole were dissolved in 5 ml THF and treated at room temperature with 7 mg of sodium hydride. The mixture was briefly stirred and subsequently 35 mg of methyl iodide were added. The mixture was stirred overnight at room temperature, treated with water and extracted several times with ethyl acetate. The organic phase was washed with water, dried and concentrated. The crude product was purified by column chromatography with cyclohexane/ethyl acetate. 67 mg of the desired product (70%, melting point 98° C.) were obtained thereby.

### EXAMPLE 7

### Synthesis of 1-[rel-(2S,3R)-3-(2-chlorophenyl)-2-(3-fluorophenyl)oxiran-2-ylmethyl]-5-(methylsulfonyl)-1H-1,2,4-triazole

A mixture of 1-[rel-(2S,3R)-3-(2-chlorophenyl)-2-(3-fluorophenyl)oxiran-2-ylmethyl]-5-(methylthio)-1H-1,2,4-triazole (352 mg, 0.94 mmol) and m-CPBA (632 mg, 2.81 mmol) in CH₂Cl₂ (8 ml) was stirred at room temperature for 20 hours. The reaction mixture was washed with 1N sodium hydroxide solution (twice 20 ml each) and saturated sodium chloride solution (twice 20 ml each), dried with sodium sulfate and freed from the solvent. The residue was purified by column chromatography (silica gel, 5:1 hexane/ethyl acetate). The target compound (315 mg, 82%) was obtained as a white solid.

1H NMR (300 MHz, CDCl3) δ 7.89 (s, 1H), 7.59-7.14 (m, 7H), 7.06-6.94 (m, 1H), 5.30 (d, 1H), 4.31 (d, 1H), 4.20 (s, 1H), 3.11 (s, 3H).

### EXAMPLE 8

### Synthesis of S-1-[rel-(2S,3R)-3-(2-chlorophenyl)-2-(3-fluorophenyl)oxiran-2-ylmethyl]-1H-1,2,4-triazole-5-yl ethanethioate

Acetyl chloride (32 μl, 0.45 mmol) was added to a mixture of 1-[rel-(2S,3R)-3-(2-chlorophenyl)-2-(3-fluorophenyl)oxiran-2-ylmethyl]-1H-1,2,4-triazole-5(4H)-thione (110 mg, 0.30 mmol) and triethylamine (83 μl, 0.60 mmol) in dry tetrahydrofuran (THF, 5 ml) dropwise at 0° C. and the mixture was stirred at this temperature for 2 hours. The target compound (130 mg, 100%) was obtained as a brown oil.

1H NMR (300 MHz, CDCl3) δ 8.29 (s, 1H), 7.66-7.20 (m, 7H), 7.04-6.90 (m, 1H), 5.21 (d, 1H), 4.11 (s, 1H), 3.72 (d, 1H), 2.89 (s, 3H).

### EXAMPLE 9

### Synthesis of 1-[rel-(2S,3R)-3-(2-chlorophenyl)-2-(3-fluorophenyl)oxiran-2-ylmethyl]-5-thiocyanato-1H-1,2,4-triazole

A mixture of 1-[rel-(2S,3R)-3-(2-chlorophenyl)-2-(3-fluorophenyl)oxiran-2-ylmethyl]-1H-1,2,4-triazole-5(4H)-thione (200 mg, 0.6 mmol), triethylamine (152 μl, 1.1 mmol) and cyanogen bromide BrCN (88 mg, 0.8 mmol) in dry tetrahydrofuran (THF, 10 ml) was slowly stirred at room temperature for 2 hours. Ethyl acetate (20 ml) was added and the mixture was washed with saturated sodium chloride solution (three times 20 ml each), dried with sodium sulfate and freed from the solvent. The residue was purified by column chromatography (silica gel, 10:1 hexane/ethyl acetate). The target compound (120 mg, 56%) was obtained as a white solid.

1H NMR (300 MHz, CDCl3) δ 7.82 (s, 1H), 7.59-7.00 (m, 8H), 4.88 (d, 1H), 4.21 (s, 1H), 4.12 (d, 1H).

### EXAMPLE 10

### Synthesis of S-1-[rel-(2S,3R)-3-(2-chlorophenyl)-2-(3-fluorophenyl)oxiran-2-ylmethyl]-1H-1,2,4-triazole-5-yl O,O-diethyl phosphorodithioate

P(S)(OEt)₂Cl (131 μL, 0.83 mmol) and triethylamine (152 μL, 1.1 mmol) and a catalytic amount of DMAP were added dropwise to a solution of 1-[rel-(2S,3R)-3-(2-chlorophenyl)-2-(3-fluorophenyl)oxiran-2-ylmethyl]-1H-1,2,4-triazole-5(4H)-thione (200 mg, 0.55 mmol) in dry tetrahydrofuran (THF, 5 ml). The reaction mixture was stirred at 0° C. for 2 hours. The reaction mixture was treated with saturated sodium chloride solution (30 ml) and then extracted with ethyl acetate (20 ml). The organic phase was washed with saturated sodium chloride solution (three times 20 ml each), dried with sodium sulfate and freed from the solvent. The residue was purified by column chromatography (silica gel, 3:1 hexane/ethyl acetate). The target compound (145 mg, 51%) was obtained as a colorless oil.

1H NMR (300 MHz, CDCl3) δ 8.00 (s, 1H), 7.60-7.20 (m, 7H), 6.99-6.87 (m, 1H), 5.19 (d, 1H), 4.31-4.01 (m, 5H), 3.62 (d, 1H), 1.28 (dt, 6H).

### EXAMPLE 11

### Synthesis of 1,2-bis[1-rel-(2S,3R)-3-(2-chlorophenyl)-2-(3-fluorophenyl)oxiran-2-ylmethyl-1H-1,2,4-triazole-5-yl]disulfane

A mixture of 1-[rel-(2S,3R)-3-(2-chlorophenyl)-2-(3-fluorophenyl)oxiran-2-ylmethyl]-1H-1,2,4-triazole-5(4H)-thione (150 mg, 0.42 mmol) and iodine (63 mg, 0.25 mmol) in dry tetrahydrofuran (THF, 5 ml) was stirred at room temperature for 4 hours. The residue was purified by preparative thin-layer chromatography. The target compound (60 mg, 40%) was obtained as a white solid (melting point 65-68° C.).

### EXAMPLE 12

### Synthesis of methyl {2-[rel-(2S,3R)-3-(2-chlorophenyl)-2-(3-fluorophenyl)-oxiranylmethyl]-2H-[1,2,4]triazole-3-yl}thiocarbonate

150 mg of 1-[rel-(2S,3R)-3-(2-chlorophenyl)-2-(3-fluorophenyl)oxiran-2-ylmethyl]-1H-1,2,4-triazole-5(4H)-thione were dissolved in 5 ml of THF and the mixture was treated at room temperature with 7 mg of sodium hydride. It was briefly stirred and subsequently 24 mg of methyl chloroformate were added. The mixture was stirred overnight at room temperature gerührt, treated with water and extracted several times with ethyl acetate. The organic phase was washed with water, dried and concentrated. The crude product was purified by column chromatography with cyclohexane/ethyl acetate. 50 mg of the desired product (51%) were obtained thereby.

HPLC-MS: 3.682 min (m/z: 420)

### Biological Experiments

### Greenhouse

### Active Compound Preparation

The active compounds were prepared separately or together as a stock solution with 25 mg of active compound, which was made up to 10 ml with a mixture of acetone and/or DMSO and the emulsifier Uniperol® EL (wetting agent with emulsifying and dispersant action based on ethoxylated alkylphenols) in the volume ratio solvent/emulsifier of 99 to 1. Subsequently, it was made up to 100 ml with water. This stock solution was diluted with the described solvent/emulsifier/water mixture to the active compound concentration indicated below. Alternatively to this, the active compounds were used as a commercially available ready-to-use solution and diluted with water to the active compound concentration indicated.

### USE EXAMPLE 1

### Curative Activity Against Soya Rust Caused by Phakopsora pachyrhizi (Phakpa K1)

Leaves of soya seedlings grown in pots were inoculated with a spore suspension of soya rust (Phakpsora pachyrhizi). The pots were then placed in a chamber of high atmospheric humidity (90 to 95%) and 23 to 27° C. for 24 hours. During this time, the spores germinated and the germ tubes penetrated into the leaf tissue. The infected plants were then sprayed with the active compound solution described above in the active compound concentration indicated below until dripping wet. After the drying of the spray coating, the test plants were cultivated in a greenhouse at temperatures between 23 and 27° C. and 60 to 80% relative atmospheric humidity for 14 days. The extent of the rust fungus development on the leaves was then determined visually in % attack.

| | | Attack in % |
|---|---|---|
| Compound | | at 600 ppm |
| No. | Structure | with Phakpa K1 |
| 6 | 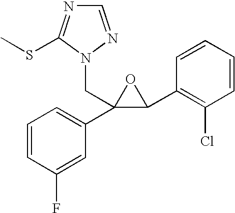 | 0 |
| untreated | | 90 |

### Microtest

The active compounds were formulated separately or together as a stock solution with a concentration of 10 000 ppm in DMSO.

### USE EXAMPLE NO. 2

### Activity Against the Causative Organism of Septoria Leaf Blotch Septoria tritici in the Microtiter Test (Septtr)

The stock solution was pipetted into a microtiter plate (MTP) and diluted with water to the indicated active compound concentration. Subsequently, the addition of an aqueous spore suspension based on malt of Septoria tritici took place. The plates were mounted in a water vapor-saturated chamber at temperatures of 18° C. The MTPs on the 7th day after the inoculation were measured at 405 nm with an absorption photometer.

The measured parameters were set off against the growth of the active compound-free control variant (100%) and the fungus- and active compound-free blank value in order to determine the relative growth in % of the pathogens in the individual active compounds.

| | | Growth in % |
|---|---|---|
| Compound | | at 31 ppm |
| No. | Structure | with Septtr |
| 1 | 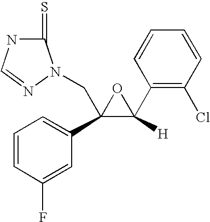 | 21 |
| 3 | 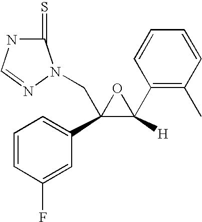 | 11 |
| 9 | 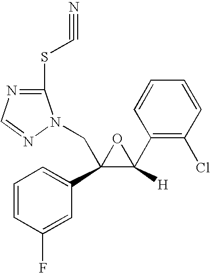 | 49 |

### USE EXAMPLE NO. 3

### Activity Against the Causative Organism of Rice Blast Pyriculana oryzae in the Microtiter Test (Pyrior)

The stock solution was pipetted into a microtiter plate (MTP) and diluted with water to the indicated active compound concentration. Subsequently the addition of an aqueous spore suspension based on malt of Pyriculana oryzae took place. The plates were mounted in a water vapor-saturated chamber at temperatures of 18° C. The MTPs on the seventh day after the inoculation were measured at 405 nm with an absorption photometer.

The measured parameters were set off against the growth of the active compound-free control variant (=100%) and the fungus and active compound-free blank value in order to determine the relative growth in % of the pathogens in the individual active compounds.

| | | Growth in % |
|---|---|---|
| Compound | | at 31 ppm |
| No. | Structure | with Pyrior |
| 4 | 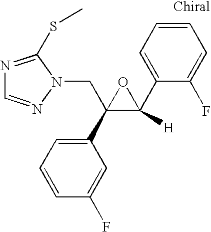 | 0 |
| 10 | 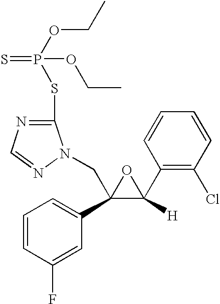 | 0 |
| 5 | 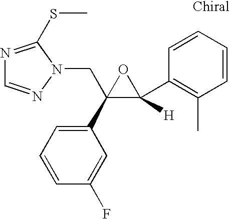 | 0 |

### USE EXAMPLE NO. 4

### Activity Against the Causative Organism of Glume Blotch, Leptosphaeria nodorum in the Microtiter Test (Leptno)

The stock solution is pipetted into a microtiter plate (MTP) and diluted with water to the indicated active compound concentration. Subsequently, the addition of an aqueous spore suspension based on malt of Leptosphaeria nodorum takes place. The plates were mounted in a water vapor-saturated chamber at temperatures of 18° C. The MTPs were measured on the 7th day after the inoculation at 405 nm with an absorption photometer.

The measured parameters were set off against the growth of the active compound-free control variant and the fungus- and active compound-free blank value in order to determine the relative growth in % of the pathogens in the individual active compounds.

| | | Growth in % |
|---|---|---|
| Compound | | at 31 ppm |
| No. | Structure | with Leptno |
| 12 | 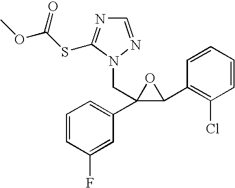 | 0 |

### Comparison Tests

| | | Growth in | Growth in | Growth in |
|---|---|---|---|---|
| | | % at | % at | % at |
| Compound | | 31 ppm | 31 ppm | 31 ppm |
| No. | Structure | with Septtr | with Pyrior | with Leptno |
| 1 | 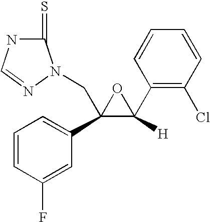 | 21 | | |
| WO9638440, p. 6 (6th Compound), p. 143 (Example 29, II-17) | 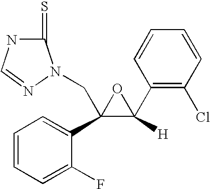 | 58 | | |
| 4 | 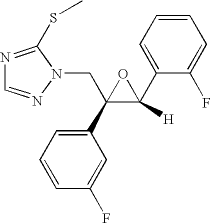 | | 0 | |
| WO9638440, p. 17 (4th compound), | 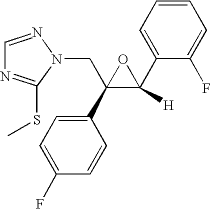 | | 100 | |
| 12 | 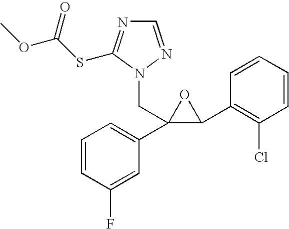 | 25 | | 0 |
| WO9742178, p. 55 (4th compound) | 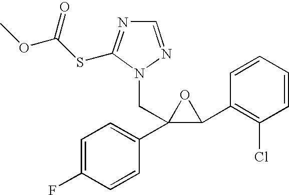 | 100 | | 100 |

1-26. (canceled)

27. An azolylmethyloxirane of the formula I 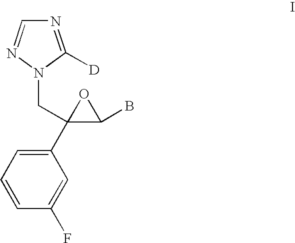
in which:
B is phenyl which is unsubstituted or substituted by one, two, three or four identical or different substituents L, wherein
L is halogen, cyano, nitro, cyanato (OCN), C₁-C₈-alkyl, C₁-C₈-haloalkyl, phenyl-C₁-C₆-alkyloxy, C₂-C₈-alkenyl, C₂-C₈-haloalkenyl, C₂-C₈-alkynyl, C₂-C₈-haloalkynyl, C₄-C₁₀-alkadienyl, C₄-C₁₀-haloalkadienyl, C₁-C₈-alkoxy, C₁-C₈-haloalkoxy, C₁-C₈-alkylcarbonyloxy, C₁-C₈-alkylsulfonyloxy, C₂-C₈-alkenyloxy, C₂-C₈-haloalkenyloxy, C₂-C₈-alkynyloxy, C₂-C₈-haloalkynyloxy, C₃-C₈-cycloalkyl, C₃-C₈-halocycloalkyl, C₃-C₈-cycloalkenyl, C₃-C₈-halocycloalkenyl, C₃-C₈-cycloalkoxy, C₃-C₆-cyclo-alkenyloxy, hydroxyimino-C₁-C₈-alkyl, C₁-C₆-alkylene, oxy-C₂-C₄-alkylene, oxy-C₁-C₃-alkyleneoxy, C₁-C₈-alkoximino-C₁-C₈-alkyl, C₂-C₈-alkenyloximino-C₁-C₈-alkyl, C₂-C₈-alkynyloximino-C₁-C₈-alkyl, S(═O)ₙA¹, C(═O)A², C(═S)A², NA³A⁴, phenyl, phenyloxy or a five- or six-membered saturated, partially unsaturated or aromatic heterocycle which comprises one, two, three or four heteroatoms selected from the group consisting of O, N and S; wherein
n is 0, 1 or 2;
A¹ is hydrogen, hydroxy, C₁-C₈-alkyl, C₁-C₈-haloalkyl, amino, C₁-C₈-alkylamino or di-C₁-C₈-alkylamino,
A² is one of the groups mentioned for A¹ or C₂-C₈-alkenyl, C₂-C₈-haloalkenyl, C₂-C₈-alkynyl, C₂-C₈-haloalkynyl, C₁-C₈-alkoxy, C₁-C₈-haloalkoxy, C₂-C₈-alkenyloxy, C₂-C₈-haloalkenyloxy, C₂-C₈-alkynyloxy, C₂-C₈-haloalkynyloxy, C₃-C₈-cycloalkyl, C₃-C₈-halocycloalkyl, C₃-C₈-cycloalkoxy or C₃-C₈-halocycloalkoxy;
A³, A⁴ independently of one another are hydrogen, C₁-C₈-alkyl, C₁-C₈-haloalkyl, C₂-C₈-alkenyl, C₂-C₈-haloalkenyl, C₂-C₈-alkynyl, C₂-C₈-haloalkynyl, C₃-C₈-cycloalkyl, C₃-C₈-halocycloalkyl, C₃-C₈-cyclo-alkenyl or C₃-C₈-halocycloalkenyl;
where the aliphatic and/or alicyclic and/or aromatic groups of the radical definitions of L for their part may carry one, two, three or four identical or different groups R^{L}:
R^{L} is halogen, cyano, nitro, C₁-C₈-alkyl, C₁-C₈-haloalkyl, C₁-C₈-alkoxy, C₁-C₈-haloalkoxy, C₃-C₈-cycloalkyl, C₃-C₈-halocycloalkyl, C₃-C₈-cycloalkenyl, C₃-C₈-cycloalkoxy, C₃-C₈-halocycloalkoxy, C₁-C₈-alkylcarbonyl, C₁-C₈-alkylcarbonyloxy, C₁-C₈-alkoxycarbonyl, amino, C₁-C₈-alkylamino, or di-C₁-C₈-alkylamino;
D -is S—R, where
R is hydrogen, C₁-C₈-alkyl, C₁-C₈-haloalkyl, C₂-C₈-alkenyl, C₂-C₈-haloalkenyl, C₂-C₈-alkynyl, C₂-C₈-haloalkynyl, C(═O)R³, C(═S)R³, SO₂R⁴ or CN; where
R³ is hydrogen, C₁-C₈-alkyl, C₁-C₈-haloalkyl, C₁-C₈-alkoxy, C₁-C₈-haloalkoxy or NA³A⁴; and
R⁴ is C₁-C₈-alkyl, phenyl-C₁-C₈-alkyl or phenyl, where the phenyl groups are in each case unsubstituted or substituted by one, two or three groups independently selected from the group consisting of halogen and C₁-C₄-alkyl;
is a group DI 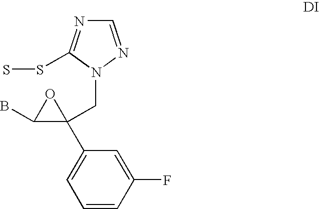
where B is as defined above;
is a group DII 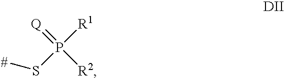
where # is the point of attachment to the triazolyl ring and Q, R¹ and R² are as defined below:
Q is O or S;
R¹, R² independently of one another are C₁-C₈-alkyl, C₁-C₈-haloalkyl, C₁-C₈-alkoxy, C₁-C₈-alkoxy-C₁-C₈-alkoxy, C₁-C₈-haloalkoxy, C₁-C₈-alkoxy-C₁-C₈-alkyl, C₁-C₈-alkylthio, C₂-C₈-alkenylthio, C₂-C₈-alkynylthio, C₃-C₈-cycloalkyl, C₃-C₈-cycloalkylthio, phenyl, phenyl-C₁-C₄-alkyl, phenoxy, phenylthio, phenyl-C₁-C₄-alkoxy or NR⁵R⁶, where R⁵ is H or C₁-C₈-alkyl and R⁶ is C₁-C₈-alkyl, phenyl-C₁-C₄-alkyl or phenyl or R⁵ and R⁶ together are an alkylene chain having four or five carbon atoms or form a radical of the formula —CH₂—CH₂—O—CH₂—CH₂— or —CH₂—CH₂—NR⁷—CH₂—CH₂— in which R⁷ is hydrogen or C₁-C₄-alkyl; where the aromatic groups in the radicals mentioned above are in each case independently of one another unsubstituted or substituted by one, two or three groups selected from the group consisting of halogen and C₁-C₄-alkyl;
or
is a group SM, where M is as defined below:
M is an alkali metal cation, an equivalent of an alkaline earth metal cation, an equivalent of a copper, zinc, iron or nickel cation or an ammonium cation of the formula (E) 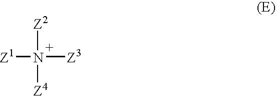
in which
Z¹ and Z² independently are hydrogen or C₁-C₈-alkyl;
Z³ and Z⁴ independently are hydrogen, C₁-C₈-alkyl, benzyl or phenyl; where the phenyl groups are in each case unsubstituted or substituted by one, two or three groups independently selected from the group consisting of halogen and C₁-C₄-alkyl;
and an agriculturally acceptable salt thereof.

28. The compound according to claim 27 where B is phenyl which comprises exactly one substituent L.

29. The compound according to claim 27 in which B is phenyl which comprises two or three independently selected substituents L.

30. The compound according to claim 29 where at least one substituent L is located in the ortho-position to the point of attachment of the phenyl ring to the oxirane ring.

31. The compound according to claim 27 where L is in each case independently selected from the group consisting of halogen, C₁-C₄-alkyl, C₁-C₄-haloalkyl, C₁-C₄-alkoxy, C₁-C₄-haloalkoxy and C₁-C₄-haloalkylthio.

32. The compound according to claim 31 where L is in each case independently selected from the group consisting of F, Cl, CH₃, C₂H₅, CF₃, OCH₃, OC₂H₅, OCF₃, OCHF₂ and SCF₃.

33. The compound according to claim 27 where D is SH or S—C₁-C₄-alkyl.

34. The compound according to claim 27 where D is S—C(═O)R³ and R³ is C₁-C₄-alkyl.

35. A composition comprising a compound of claim 27 and/or a salt thereof.

36. The composition according to claim 35 which further comprises at least one solid or liquid carrier.

37. The composition according to claim 35 which comprises at least one further fungicidally, insecticidally and/or herbicidally active compound.

38. A seed comprising at least one compound of the formula I according to claim 27 and/or an agriculturally acceptable salt thereof.

39. A method for controlling phytopathogenic fungi which comprises treating the fungi or the materials, plants, the soil or seed to be protected against fungal attack with an effective amount of a compound of the formula I according to claim 27 or an agriculturally acceptable salt thereof.

40. A medicament comprising at least one compound of the formula I according to claim 27 and/or a pharmaceutically acceptable salt thereof.

41. A process for preparing an antimycotic which comprises mixing at least one compound of the formula I according to claim 27 with a pharmaceutically acceptable salt thereof.

42. A process for preparing compounds of the formula I according to claim 27 in which D is SR where R is hydrogen, C₁-C₈-alkyl, C₂-C₈-alkenyl, C₂-C₈-haloalkenyl, C₂-C₈-alkynyl or C₂-C₈-haloalkynyl, which comprises
(a1) reacting a compound IIIb 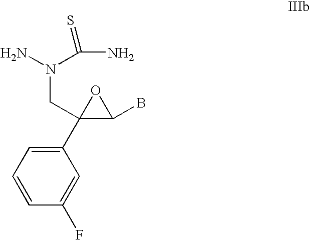
with formic acid to give compounds I-1; 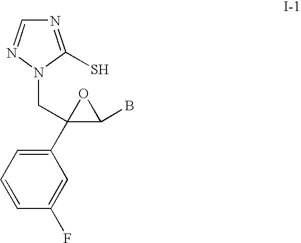
wherein D is SH and, if desired to obtain compounds I in which D is SR where R is C₁-C₈-alkyl, C₁-C₈-haloalkyl, C₂-C₈-alkenyl, C₂-C₈-haloalkenyl, C₂-C₈-alkynyl or C₂-C₈-haloalkynyl,
(b1) reacting compounds I-1 with R—X where X is halogen or tri-fluoro(C₁-C₈)alkylsulfonate.

43. A process for preparing compounds of the formula IIIb 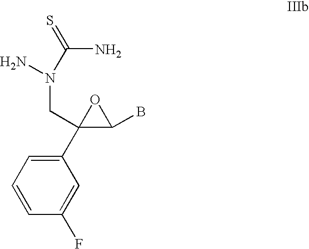
wherein B is as defined in claim 27,
which comprises
(a2) reacting a compound of the formula IIIa 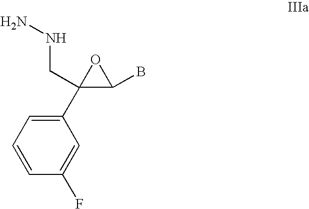
with a thiocyanate YSCN in which Y is an alkali metal or ammonium.

44. A process for preparing compounds I according to claim 27 in which D is SR where R is hydrogen, C₁-C₈-alkyl, C₁-C₈-haloalkyl, C₂-C₈-alkenyl, C₂-C₈-haloalkenyl, C₂-C₈-alkynyl or C₂-C₈-haloalkynyl, which comprises
(a3) oxidizing a compound of the formula IIIc 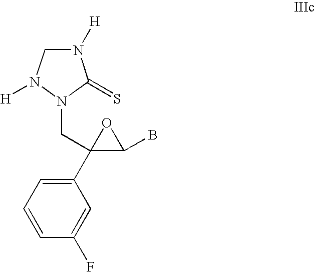
to give a compound I-1; 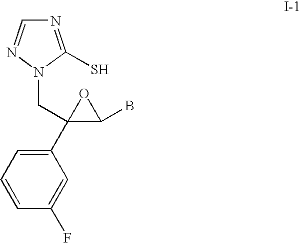
in which D is SH; and, if desired to obtain compounds I in which D is SR where R is C₁-C₈-alkyl, C₁-C₈-haloalkyl, C₂-C₈-alkenyl, C₂-C₈-haloalkenyl, C₂-C₈-alkynyl or C₂-C₈-haloalkynyl,
(b3) reacting compounds I-1 with R—X where X is halogen or tri-fluoro(C₁-C₈)alkylsulfonate.

45. A process for preparing compounds I according to claim 27 in which D is SR where R is hydrogen, C₁-C₈-alkyl, C₁-C₈-haloalkyl, C₂-C₈-alkenyl, C₂-C₈-haloalkenyl, C₂-C₈-alkynyl or C₂-C₈-haloalkynyl, which comprises
(a4) reacting a compound IIId 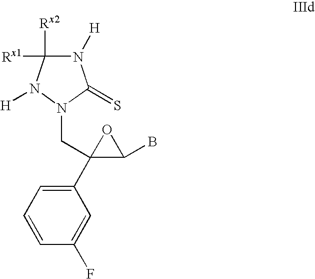
where R^{x1} is C₁-C₄-alkyl or phenyl, R^{x2} is hydrogen or C₁-C₄-alkyl, or R^{x1} and R^{x2} together form a —(CH₂)₅— chain with formic acid to obtain compounds I—; 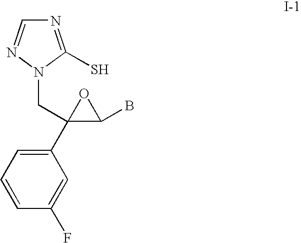
in which D is SH; and, if desired to obtain compounds I in which D is SR where R is C₁-C₈-alkyl, C₁-C₈-haloalkyl, C₂-C₈-alkenyl, C₂-C₈-haloalkenyl, C₂-C₈-alkynyl or C₂-C₈-haloalkynyl,
(b4) reacting compounds I-1 with R—X where X is halogen or trifluoro(C₁-C₈)alkylsulfonate.

46. A compound of formula IIIa, 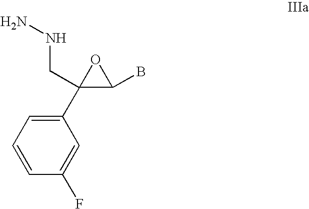
formula IIIb, 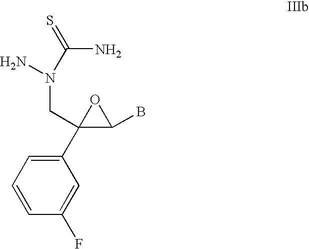
formula IIIc 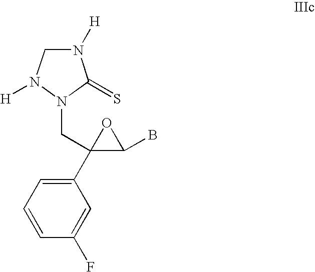
or formula IIId 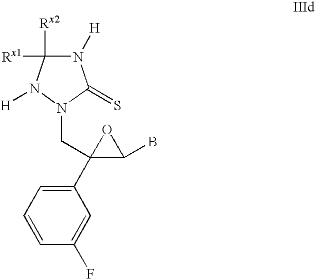
wherein
B is as defined in claim 27,
or an agriculturally acceptable salt thereof.

47. A process for preparing compounds I according to claim 27 in which D is SR where R is hydrogen, C₁-C₈-alkyl, C₁-C₈-haloalkyl, C₂-C₈-alkenyl, C₂-C₈-haloalkenyl, C₂-C₈-alkynyl or C₂-C₈-haloalkynyl, which comprises
(a5) reacting of a compound of the formula II 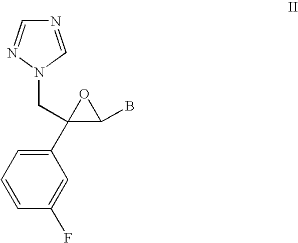
with a strong base and sulfur powder to give a compound I-1; 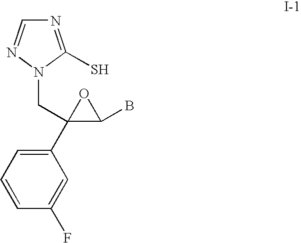
in which D is SH; and, if desired to obtain compounds I in which D is SR where R is C₁-C₈-alkyl, C₁-C₈-haloalkyl, C₂-C₈-alkenyl, C₂-C₈-haloalkenyl, C₂-C₈-alkynyl or C₂-C₈-haloalkynyl,
(b5) reacting compounds I-1 with R—X where X is halogen or tri-fluoro(C₁-C₈)alkylsulfonate.

48. A process for preparing compounds of the formula II, 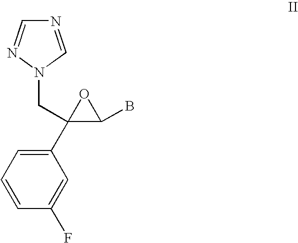
wherein B is as defined in claim 27,
which comprises
(a6) reacting a compound of the formula III 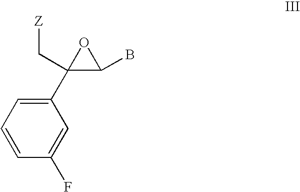
in which Z is a leaving group, with 1,2,4-triazole and a base.

49. The compound of the formula III, 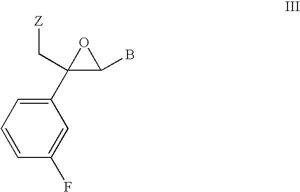
in which B is as defined in claim 27 and Z is a leaving group, except for the compounds
anti-2-(3-fluorophenyl)-2-(chloromethyl)-3-(2-chlorophenyl)oxirane,
anti-2-(3-fluorophenyl)-2-(chloromethyl)-3-(4-chlorophenyl)oxirane,
anti-2-(3-fluorophenyl)-2-(chloromethyl)-3-(3-chlorophenyl)oxirane,
anti-2-(3-fluorophenyl)-2-(chloromethyl)-3-(4-fluorophenyl)oxirane,
2-(3-fluorophenyl)-2-(bromomethyl)-3-(2-methylphenyl)oxirane,
2-(3-fluorophenyl)-2-(CH₃O₂SO-methyl)-3-(2-methylphenyl)oxirane and
2-hydroxymethyl-2-(3-fluorophenyl)-3-(2-methylphenyl)oxirane.

50. The compound III according to claim 49 in which B is not ortho-methylphenyl.
